(19) United States
(12) Patent Application Publication
(10) Pub. No.: US 2026/0184784 A1
(43) Pub. Date: Jul. 2, 2026

(54) ANTIBODIES FOR MULTIPLE MYELOMA TREATMENT

(71) Applicants: THE TRUSTEES OF INDIANA UNIVERSITY, Bloomington, IN (US); Lonza Walkersville, Inc., Walkersville, MD (US)

(72) Inventors: Fabiana Perna, Tampa, FL (US); Tamara Jatoba Laskowski, Kingwood, TX (US)

(21) Appl. No.: 19/341,097

(22) Filed: Sep. 26, 2025

Related U.S. Application Data
(60) Provisional application No. 63/699,547, filed on Sep. 26, 2024.

(51) Int. Cl. C07K 16/28

(52) U.S. Cl. CPC C07K 16/2803; C07K 16/283; C07K 2317/31

(57) ABSTRACT

Cell surface polypeptides SEMA-4A and FCRL3 have been identified as being associated with multiple myeloma. Accordingly, these peptides represent targets for immuno-based therapeutic strategies for treating multiple myeloma. Antibodies that specifically bind to one of these polypeptides can be used to treat multiple myeloma. Accordingly, new compositions and methods utilizing novel antibodies that target SEMA-4A and FCRL3 are described.

### CROSS-REFERENCE TO RELATED APPLICATIONS

This application claims priority to U.S. Provisional Patent Application No. 63/699,547 filed on Sep. 26, 2024, the disclosure of which is expressly incorporated herein.

### INCORPORATION BY REFERENCE OF MATERIAL SUBMITTED ELECTRONICALLY

The sequence listing associated with this application is provided in XML format in lieu of a paper copy, and is hereby incorporated by reference into the specification. The name of the XML file containing the sequence listing is “29920-429094—Antibodies for multiple myeloma treatment.xml”. The XML file is 339,758 bytes, and was created on Sep. 5, 2025, and is being submitted electronically, concurrent with the filing of this application.

### BACKGROUND

Multiple myeloma (MM), also known as plasma cell myeloma, is a cancer of plasma cells, a type of white blood cell that normally produces antibodies. Globally, multiple myeloma affected 488,000 people and resulted in 101,100 deaths in 2015. In the United States, it develops in 6.5 per 100,000 people per year and 0.7% of people are affected at some point in their lives. Without treatment, typical survival is seven months. With current treatments, survival is usually 4-5 years.

Targeting tumor antigens with immunotherapy is rapidly emerging as a promising approach for cancer treatment. This is based on successes of antibody-mediated checkpoint blockade and engineered T cells. In MM, monoclonal antibodies, antibody-drug conjugates, bi-specific antibody constructs, and Chimeric Antigen Receptor (CAR) T-cell therapy targeting BCMA (B-cell maturation antigen) are significantly improving survival in patients with MM. Data from >20 clinical trials involving anti-BCMA CAR T cells have demonstrated that patients with relapsed and/or refractory MM can achieve objective responses.

While tremendous progress in the treatment of MM has been made over the past 25 years, myeloma remains an incurable disease, with a particularly poor prognosis for patients with refractory relapsed MM (RRMM) or high-risk cytogenetics. Monoclonal antibodies (mAbs), which typically possess both a targeting function and an ability to trigger biological mechanisms for the destruction of their binding targets, can be particularly useful as therapeutic agents for B cell depletion. Cell surface polypeptides such as SEMA-4A and FCRL3 have been identified as being associated with MM cells. Accordingly, these peptides represent targets for immuno-based therapeutic strategies for treating MM. Antibodies that specifically bind to one of these polypeptides can be used to treat MM.

Accordingly, the present disclosure provides new compositions and methods utilizing novel antibodies that target SEMA-4A and FCRL3. As described herein, novel antibodies were identified that can bind SEMA-4A and FCRL3 on the surface of tumor cell lines and patient cells. The antibodies of the present disclosure provide for efficient ways to target tumor cells in different stages of disease, sourcing options that current approaches do not currently support. Further, the antibodies of the present disclosure can be used for the development of combination therapy strategies.

Other objects, features, and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### SUMMARY

In one aspect, the present disclosure is directed to the development of an antigen-binding molecule that bind to SEMA-4A. The antigen-binding molecule comprises an immunoglobulin heavy chain domain (HC) and an immunoglobulin light chain domain (LC), wherein the HC or LC comprises a complementarity determining region. In some embodiments, the HC comprises an amino acid sequence determined by the genetic sequence of one of SEQ ID NO: 11-19. In some embodiments, the LC comprises an amino acid sequence determined by the genetic sequence of one of SEQ ID NO: 1-10 as listed in Tables 5-10.

In a second aspect, the present disclosure is directed to the development of an antigen-binding molecule that bind to FCRL3. The antigen-binding molecule comprises an immunoglobulin heavy chain domain (HC) and an immunoglobulin light chain domain (LC), wherein the HC or LC comprises a complementarity determining region. In some embodiments, the HC comprises an amino acid sequence determined by the genetic sequence of one of SEQ ID NO: 31-40. In some embodiments, the LC comprises the amino acid sequence determined by the genetic sequence of one of SEQ ID NO: 20-30 as listed in Tables 5-10.

In a third aspect, the present disclosure is directed to a pharmaceutical composition comprising an antigen-binding molecule that binds to SEMA-4A or FCRL3. In some embodiments, the pharmaceutical composition comprises an antigen-binding molecule that includes an immunoglobulin heavy chain domain (HC) and an immunoglobulin light chain domain (LC), wherein the HC or LC comprises a complementarity determining region. In some embodiments, the HC comprises an amino acid sequence determined by the genetic sequence of one of SEQ ID NO: 11-19. In some embodiments, the LC comprises an amino acid sequence determined by the genetic sequence of one of SEQ ID NO: 1-10 as listed in Tables 5-10. In some embodiments, the HC comprises an amino acid sequence determined by the genetic sequence of one of SEQ ID NO: 31-40. In some embodiments, the LC comprises the amino acid sequence determined by the genetic sequence of one of SEQ ID NO: 20-30 as listed in Tables 5-10.

In a fourth aspect, the present disclosure is directed to a method for treating multiple myeloma. In some embodiments, the method comprises administering a pharmaceutical composition comprising an antigen-binding molecule that binds to SEMA-4A or FCRL3 to a patient in need of such treatment.

### BRIEF DESCRIPTION OF THE DRAWINGS

FIG. 1 illustrates a binning experiment where single antigen-binding molecules that specifically bind to an antigen are chemically conjugated to a chip and other antigen-binding molecules that specifically bind to the same antigen are individually added in solution.

FIG. 2 illustrates a sensor gram that represents a single antigen-binding molecule (e.g., antibody) that has been chemically conjugated to the chip. Each individual trace represents an antigen that has been added to the chip

FIG. 3A illustrates a binning experiment for antibodies targeting SEMA-4A.

FIG. 3B illustrates the positive controls, Anti-His antibodies in the binning experiment and one bin (grey 1) of antibodies that block each other.

FIG. 3C illustrates one bin of the binning experiment with two bins of SEMA-4A antibodies.

FIG. 3D illustrates that 1F5 may bind to the antigen if it is first to bind the antigen but may no longer be able to bind to SEMA-4A if other antibodies bind to SEMA-4A first.

FIG. 4 illustrates a binning experiment for antibodies targeting FCRL3.

FIG. 5A shows the mean fluorescence intensity (MFI) values from flow-cytometric analyses of antibodies targeting SEMA-4A.

FIG. 5B shows the representative FACS plots with three antibodies with different binding strength.

FIG. 6 shows the shows the MFI values from flow-cytometric analyses of antibodies targeting FCRL3.

### DETAILED DESCRIPTION

### Definitions

Unless defined otherwise, all technical and scientific terms used herein have the same meanings as commonly understood by one of ordinary skill in the art to which this invention belongs.

The term “about” as used herein means greater or lesser than the value or range of values stated by 10 percent but is not intended to designate any value or range of values to only this broader definition. Each value or range of values preceded by the term “about” is also intended to encompass the embodiment of the stated absolute value or range of values.

As used, herein the terms “native” or “natural” define a condition found in nature. A “native DNA sequence” is a DNA sequence present in nature that was produced by natural means but not generated by genetic engineering (e.g., using molecular biology/transformation techniques)

The term “linked” or like terms refers to the connection between two groups. The linkage may comprise a covalent, ionic, or hydrogen bond or other interaction that binds two compounds or substances to one another.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “Therapeutic agent,” “pharmaceutical agent” or “drug” refer to any therapeutic or prophylactic agent which may be used in the treatment (including the prevention, diagnosis, alleviation, or cure) of a malady, affliction, disease or injury in a patient.

As used herein, the term “pharmaceutically acceptable carrier” includes any of the standard pharmaceutical carriers, such as a phosphate-buffered saline solution, water, emulsions such as an oil or water or water/oil emulsion, and various types of wetting agents. The term also encompasses any of the agents approved by a regulatory agency of the US Federal government or listed in the US Pharmacopeia for use in animals, including humans.

As used herein, the term “parenteral” includes administration subcutaneously, intravenously, or intramuscularly.

As used herein, an “antigen-binding molecule” refers to a molecule that has a binding affinity for a target antigen. It will be understood that this term extends to immunoglobulins, immunoglobulin fragments, and non-immunoglobulin-derived protein frameworks that exhibit antigen-binding activity. Illustrative examples of suitable antigen-binding molecules include antibodies and antigen-binding fragments thereof. Preferably, the antigen-binding molecule binds specifically to CD20 so as to reduce its activity. The antigen-binding molecule, as described herein, can be conjugated to another molecule or moiety, including functional moieties (e.g., toxins), detectable moieties (e.g., fluorescent molecules, radioisotopes), small molecule drugs, and polypeptides.

As used herein, the term “antibody” refers to any antigen-binding molecule or molecular complex comprising at least one complementarity-determining region (CDR) that binds specifically to, or interacts specifically with, the target antigen. The term “antibody” includes full-length immunoglobulin molecules comprising two heavy (H) chains and two light (L) chains inter-connected by disulfide bonds, as well as multimers thereof (e.g., IgM). Each heavy chain (HC) comprises a heavy chain variable region and a heavy chain constant region. Each light chain (LC) comprises a light chain variable region and a light chain constant region. The HC and LC regions can comprise complementarity determining regions (CDRs), interspersed with regions that are more conserved, also referred to as framework regions (FR).

Suitable antibodies include antibodies of any class, such as IgG, IgA, or IgM (including sub-classes thereof). There are five major classes of immunoglobulins: IgA, IgD, IgE, IgG, and IgM, characterized by heavy-chain constant regions α, δ, ε, γ, and μ, respectively. Several antibody classes may be further divided into subclasses (isotypes), e.g., IgG1, IgG2, IgG3, IgG4, IgA1, and IgA2. The subunit structures and three-dimensional configurations of different classes of immunoglobulins will be well known to persons skilled in the art.

As used herein, the term “biologically active fragments” of the antibodies described herein encompasses natural or synthetic portions of the respective full-length antibody that retain the capability of specific binding to the target epitope

As used herein, the term “complementarity determining region” (CDR) refers to the region of an immunoglobulin variable domain that recognizes and binds to the target antigen.

The terms “antigen-binding fragment”, “antigen-binding portion”, “antigen-binding domain”, “antigen-binding site” and the like are used interchangeably herein to refer to a part of an antigen-binding molecule that retains the ability to bind to the target antigen, for instance, SEMA-4A or FCRL3. These terms include naturally occurring, enzymatically obtainable, synthetic, or genetically engineered (recombinant) polypeptides and glycoproteins that specifically bind to SEMA-4A or FCRL3 to form a complex. In some embodiments, the antigen-binding fragment may be comprised in a target binding modality including but not limited to an ADC, a bi-specific engager, and a tri-specific engager. Such a target-binding modality may bind to the target antigen, for instance, SEMA-4A or FCRL3. In some embodiments, the antigen-binding fragment and/or the target-binding modalities may be used in or engineered into one or more cell types including but not limited to T cells, NK cells, macrophages, and pluripotent stem cells.

Antigen-binding fragments may be derived, for example, from naturally derived immunoglobulin molecules using any suitable method known to persons skilled in the art, illustrative examples of which include proteolytic digestion or recombinant genetic engineering techniques involving the manipulation and expression of nucleic acid sequences encoding antibody variable and optionally constant domains. Suitable nucleic acid sequences are known and/or are readily available from, e.g., commercial sources, DNA libraries (including, e.g., phage-antibody libraries), or can be synthesized. The nucleic acid sequences may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of suitable antigen-binding fragments include (i) Fab fragments; (ii) F(ab′) 2 fragments; (iii) Fd fragments; (iv) Fv fragments; (v) single-chain Fv (scFv) molecules; (vi) dAb fragments; and (vii) minimal recognition units consisting of the amino acid residues that mimic the variable region of an antibody (e.g., an isolated CDR). Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), and small modular immunopharmaceuticals (SMIPs), are also encompassed by the term “antigen-binding fragment,” as used herein.

In an embodiment, an antigen-binding fragment comprises at least one immunoglobulin variable domain. The variable domain may comprise an amino acid sequence of any suitable length or composition and will generally comprise at least one CDR that is adjacent to or in frame with one or more framework sequences. Where the antigen-binding fragment comprises a variable domain in the heavy chain (HC) and a variable domain in the light chain (LC), the variable domains may be situated relative to one another in any suitable arrangement. For example, the variable region may be dimeric and contain dimers. Alternatively, the antigen-binding fragment of an antibody may contain a monomeric variable domain.

In some embodiments, an antigen-binding fragment may comprise at least one variable domain covalently linked to at least one constant domain. In any configuration of variable and constant domains, including any of the exemplary configurations listed above, the variable and constant domains may be either directly linked to one another or may be linked by a full or partial hinge or linker region. A hinge region may consist of at least two (e.g., 5, 10, 15, 20, 40, 60, or more) amino acids which result in a flexible or semi-flexible linkage between adjacent variable and/or constant domains in a single polypeptide molecule. In some embodiments, the antigen-binding fragment, as herein described, may comprise a homo-dimer or hetero-dimer (or other multimer) of any of the variable and constant domain configurations listed above in non-covalent association with one another and/or with one or more monomeric variable domains (e.g., by disulfide bond(s)). A multispecific antigen-binding molecule will typically comprise at least two different variable domains, wherein each variable domain is capable of specifically binding to a separate antigen or a different epitope on the same antigen. Any multispecific antigen-binding molecule format, including bispecific antigen-binding molecule formats, may be adapted for use in the context of an antigen-binding fragment of an antibody of the present disclosure using routine techniques available in the art.

The term “variable region” or “variable domain” refers to the domain of an immunoglobulin heavy or light chain that is involved in binding to the target antigen. The variable domains of the heavy chain and tight chain of a native immunoglobulin molecule will generally have similar structures, with each domain comprising four conserved framework regions and three hypervariable regions (HVRs). See, e.g., Kindt et. al., Kuby Immunology, 6th ed., W.H. Freeman and Co., page 91 (2007). A single variable domain may be sufficient to confer antigen-binding specificity.

As used herein, the term “humanized” or “human antibody” means that the antigen-binding molecule comprises an amino acid sequence that is compatible with humans, such that the amino acid sequence is unlikely to be seen as foreign by the immune system of a human subject. In an embodiment, the humanized antigen-binding molecule comprises one or more immunoglobulin framework regions derived from one or more human immunoglobulin molecules. In some embodiments, all of the framework regions of the humanized antigen-binding molecule will be derived from one or more human immunoglobulin molecules. The humanized antibody may optionally comprise an immunoglobulin heavy chain constant region derived from a human immunoglobulin molecule.

The phrase “specifically binds” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antigen-binding molecule binds to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules (e.g., immunoglobulins) [such that they are specifically immunoreactive with a particular antigen. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

Methods of determining binding affinity and specificity are also well known in the art (see, for example, Harlow and Lane, supra); Friefelder, “Physical Biochemistry: Applications to biochemistry and molecular biology” (W.H. Freeman and Co. 1976))

“Affinity” or “binding affinity” refers to the strength of the sum total of non-covalent interactions between a single binding site of a molecule (e.g., an antigen-binding molecule) and its binding partner (e.g., an antigen). Unless indicated otherwise, as used herein, “binding affinity” refers to intrinsic binding affinity that reflects a 1:1 interaction between members of a binding pair e.g., an antigen-binding molecule. The affinity of molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

As used herein, the term “modified antibody” includes synthetic forms of antibodies that are altered such that they are not naturally occurring, e.g., antibodies that comprise at least two heavy chain portions but not two complete heavy chains (such as domain deleted antibodies or minibodies); multispecific forms of antibodies (e.g., bispecific, trispecific, etc.) altered to bind to two or more different antigens or to different epitopes on a single antigen; heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., bivalent, tetravalent, etc., antibodies that bind to three or more copies of the same antigen).

Also disclosed herein is a pharmaceutical composition comprising the SEMA-4A or FCRL3-binding molecule, as described herein, and a pharmaceutically acceptable carrier. A “pharmaceutically acceptable carrier” means a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Representative pharmaceutically acceptable carriers include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The pharmaceutical compositions may be in a variety of forms. These include, for example, liquid, semi-solid, and solid dosage forms, such as liquid solutions (e.g., injectable and infusible solutions), dispersions or suspensions, liposomes and suppositories. The preferred form depends on the intended mode of administration and therapeutic application. Suitable pharmaceutical compositions may be administered intravenously, subcutaneously or intramuscularly. In some embodiments, the compositions are in the form of injectable or infusible solutions. A preferred mode of administration is parenteral (e.g., intravenous, subcutaneous, intraperitoneal, intramuscular). In specific embodiments, the pharmaceutical composition is administered by intravenous infusion or injection. In other embodiments, the pharmaceutical composition is administered by intramuscular or subcutaneous injection.

The phrases “parenteral administration” and “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

The pharmaceutical compositions of the invention may include an effective amount of agent (i.e., the SEMA-4A or FCRL3-binding molecule) disclosed herein. The effective amount may be a “therapeutically effective amount” or a “prophylactically effective amount.” A “therapeutically effective amount” refers to an amount effective, at dosages, and for periods of time necessary, to achieve the desired therapeutic result. A therapeutically effective amount of the agent may vary according to factors such as the disease state, age, sex, and weight of the individual, and the ability of the agent to elicit a desired response in the individual. A therapeutically effective amount is also one in which any toxic or detrimental effects of the agent are outweighed by the therapeutically beneficial effects. Alternatively, this property of a composition can be evaluated by examining the ability of the compound to inhibit, for example in in vitro by assays known to the skilled practitioner. By contrast, a “prophylactically effective amount” refers to an amount effective, at dosages, and for periods of time necessary, to achieve the desired prophylactic result. Typically, since a prophylactic dose is used in subjects prior to or at an earlier stage of disease, the prophylactically effective amount will be less than the therapeutically effective amount.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells. The term “treating” as used herein may refer to (1) delaying the appearance of one or more symptoms of the condition; (2) inhibiting the development of the condition or one or more symptoms of the condition; (3) relieving the condition, i.e., causing regression of the condition or at least one or more symptoms of the condition; and/or (4) causing a decrease in the severity of the condition or of one or more symptoms of the condition. The terms “treating”, “treatment” and the like, are used interchangeably herein to mean relieving, reducing, alleviating, ameliorating or otherwise inhibiting the condition, including one or more symptoms of the condition. The terms “prevent”, “preventing”, “prophylaxis”, “prophylactic”, “preventative” and the like are used interchangeably herein to mean preventing or delaying the onset of the condition, or the risk of developing the condition. The terms “treating”, “treatment” and the like also include relieving, reducing, alleviating, ameliorating, or otherwise inhibiting the effects of the condition for at least a period of time. It is also to be understood that the terms “treating”, “treatment” and the like do not imply that the condition, or a symptom thereof, is permanently relieved, reduced, alleviated, ameliorated or otherwise inhibited and therefore also encompasses the temporary relief, reduction, alleviation, amelioration or otherwise inhibition of the condition, or of a symptom thereof.

The terms “subject”, “patient”, ‘host” or “individual” used interchangeably herein, refer to any subject, particularly a vertebrate subject, and even more particularly a mammalian subject, for whom therapy or prophylaxis is desired. In one embodiment, the subject is a human subject.

### EMBODIMENTS

Targeting B-cell maturation antigen (BCMA) with antibody conjugates and bispecific T-cell engagers (BiTE; a unique artificial bispecific monoclonal antibody that has two linked, single-chain variable fragments, and having a 1+1 antigen-binding valency) has been demonstrated to have efficacy in treating multiple myeloma (MM). Both BCMA targeting immune-therapies were granted breakthrough status for patients with RRMM by FDA. All these trials demonstrate impressive results with the ability of anti-BCMA CAR T-cells to induce deep responses in highly pretreated RRMM, however, despite this, remissions are not sustained and the majority of patients eventually relapse. One of the mechanisms of resistance lies in the antigen loss or downregulation with the emergence of low BCMA or BCMA-negative subclones.

One of the most important determinants of the success of CAR T-cell or antigen therapy is the choice of the target antigen; an ideal target should be highly expressed on all tumor cells, on cancer stem cells, in most patients, absent in normal counterparts and most organs of the whole body. Given that, studying the myeloma surface proteome is critical to identify additional immunotherapeutic targets and understand the role of an altered surfaceome in the disease biology. In fact, surface proteins may mediate regulatory mechanisms underpinning myeloma manipulation of the bone marrow microenvironment. Developing antigen-binding molecules directed to alternative targets such as SEMA-4A and FCRL3 is crucial to providing therapeutic options to patients who failed BCMA CAR therapy.

The present disclosure is directed to novel antigen binding molecules that specifically bind to SEMA-4A or FCRL3. The present disclosure is directed to antibodies that specifically target and recognize SEMA-4A or FCRL3. In some embodiments, the antibodies are monoclonal antibodies for each of these targets. In some embodiments, the binding regions of the antibodies have been mapped. In some embodiments, the antibodies can detect and bind to SEMA-4A or FCRL3 on the surface of tumor cell lines and patient cells.

In some embodiments, the antibody sequences that comprise the scFvs are extracted from the monoclonal antibodies. The scFvs can be used for the generation of chimeric antigen receptors (CAR). The scFv can constitute the binding domain (extracellular) on the CAR and can be linked to other domains (hinge, transmembrane, intracellular co-stimulatory signaling domain, and CD3zeta domain) to form a full CAR molecule.

In some embodiments, a target-binding modality comprising an antibody or antibody fragment as its binding domain can be used to detect and bind to SEMA-4A or FCRL3 on the surface of tumor cell lines and patient cells. In some embodiments, the antibody fragment may be an ADC, bispecific engagers, or tri-specific engagers.

Various embodiments of the invention are described herein as follows. In an illustrative aspect, a first antigen-binding molecule that specifically binds to SEMA-4A is provided.

In an illustrative aspect, antigen-binding molecules that specifically bind to SEMA-4A are provided.

In at least one embodiment, antibodies generated against SEMA-4A can be used to generate an antibody single-chain variable fragment, which can then be used to prepare a chimeric antigen receptor (CAR). The antibody single-chain variable fragment is a chimeric protein made up of light (LC) and heavy (HC) chains of immunoglobins, connected with a short linker peptide. In accordance with the present disclosure, the antibodies are selected in advance for their binding ability to the target antigen, SEMA-4A. In some embodiments, the linker between the variable regions of the antibodies may consist of hydrophilic residues with stretches of glycine and serine in it for flexibility as well as stretches of glutamate and lysine for added solubility.

TABLE 1

| Antibodies | |
|---|---|
| Against SEMA- | |
| 4A | Light Chain |
| 1B8 | ATGGAGAAAGACACACTCCTGCTATGGGTCCTGCTTCTCTGGGTTCCAGG |
| (SEQ ID NO: 1) | TTCCACAGGTGACATTGTGCTGACCCAATCTCCAGCTTCTTTGGCTGTGTC |
| | TCTAGGGCAGAGGGCCACCATCTCCTGCAGAGCCAGCGAAAGTGTTGAT |
| | AATTATGGCATTAGTTTTATGAACTGGTTCCAACAGAAACCAGGACAGCC |
| | ACCCAAACTCCTCATCTATGCTGCATCCAACCAAGGATCCGGGGTCCCTG |
| | CCAGGTTTAGTGGCAGTGGGTCTGGGACAGACTTCAGCCTCAACATCCAT |
| | CCTATGGAGGAGGATGATACTGCAATGTATTTCTGTCAGCAAAGTAAGG |
| | AGGTTCCATTCACGTTCGGCTCGGGGACAAAGTTGGAAATAAAACGGGC |
| | TGATGCTGCACCAACTGTATCCATCTTCCCACCATCCAGTGAGCAGTTAA |
| | CATCTGGAGGTGCCTCAGTCGTGTGCTTCTTGAA |
| 2C1 | ATGGAGACACATTCTCAGGTCTTTGTATACATGTTGCTGTGGTTGTCTGAT |
| (SEQ ID NO: 2) | GTTGAAGGTGACATTGTGATGACCCAGTCTCACAAATTCATGTCCACATC |
| | AGTAGGAGACAGGGTCAGCATCTCCTGCAAGGCCAGTCAGGATGTGGGT |
| | ACTGCTGTTGCCTGGTATCAACATAAACCAGGACAGTCTCCTAAAGTTCT |
| | GATTTACTGGGCATCCAACCGGCACACTGGAGTCCCTGATCGCTTCACAG |
| | GCAGTGGATCTGGGACAGATTTCACTCTCACCATTACCAATGTGCAGTCT |
| | GAAGACTTGGCAGATTATTTCTGTCAGCAATATAGCTACTATCCCACGTT |
| | CGGAGGGGGGACCAAGCTGGAAATAAAGCGGGCTGATGCTGCACCAACT |
| 1A3 | ATGTCCTCTGCTCAGTTCCTTGGTCTCCTGTTGCTCTGTTTTCAAGGTGTC |
| (SEQ ID NO: 3) | AGATGTGATATCCAGATAACACAGACTACATCCTCCCTGTCTGCCTCTCT |
| | GGGAGACAGAGTCACCATCAGTTGCAGTGCAAGTCAGGGCCTTAGCAAT |
| | TATTTAAACTGGTATCAGCAGAAGCCAGATGGAACTGTTAAACTCCTGAT |
| | CTATTACACATCAATTTTACACTCAGGAGTCCCATCAAGGTTCAGTGGCA |
| | GTGGGTCTGGGACAGATTATTCTCTCACCATCAGCAACCTGGAACCTGAA |
| | GATATTGCCACTTACTATTGTMAGCAGTATAGTAAGCTTCCGTGGACGTT |
| | CGGTGGAGGCTCCAAGTTGGAAATCAAACGGGCTGATGCTGCACCAACT |
| | GTATCCATCTTCCCACCATCCAGTGAGCAGTTAACATCTGGAGGTGCCTC |
| | AGTCGTGTGCTTCTTGAACAACTTCTACCCCAGAGACATCAATGTCAAGT |
| | GGAAGATTGATGGCAGTGAACGACAAAATGGTGTCCTGAACAGTTGGAC |
| | TGATCAGGACAGCAAAGACAGCACCTACAGCATGAGCAGCACCCTCACA |
| | TTGACCAAGGACGAGTATGAACGACATAACAGCTATACCTGTGAGGCCA |
| | CT |
| 1E7 | ATGAGTGTGCCCACTCAGGTCCTGGGGTTGCTGCTGCTGTGGCTTACAGA |
| (SEQ ID NO: 4) | TGCCAGATGTGACATCCAGATGACTCAGTCTCCAGCCTCCCTATCTGTAT |
| | CTGTGGGAGAAACTGTCACCATCACATGTCGAGCAAGTGAGAATATTTA |
| | CAGTAATTTAGCATGGTATCACCAGAAACAGGGAAAATCTCCTCAACTCC |
| | TGGTCTATGCTGCAACAAACTTAGCAGATGGTGTGCCATCAAGGTTCAGT |
| | GGCAATGGATCAGGCACACAGTATTCCCTCAAGATCAACAGCCTGCAGT |
| | CTGAAGATTTTGGGAGTTATTACTGTCAACATTTTTGGGGTACTCCGTGG |
| | ACGTTCGGTGGAGGCACCAAGCTGGAAATCAAACGGGCTGATGCTGCAC |
| | CAACTGTATCCATCTTCCCACCATCCAGTGAGCAGTTAACATCTGGAGGT |
| | GCCTCAGTCGTGTGCTTCTTGAACAACTTCTACCCCAAAGACATCAATGT |
| | CAAGTGGAAGATTGATGGCAGTGAACGACAAAATGGCGTCCTGAACAGT |
| | TGGACTGA |
| 1E2 | ATGGAGACAGACACACTCCTGCTATGGGTGCTGCTGCTCTGGGTTCCAGG |
| (SEQ ID NO: 5) | TTCCACTGGTGACATTGTGCTGACACAGTCTCCTGCTTCCTTACCTGTATC |
| | TCTGGGGCAGAGGGCCACCATTTCATGTAGGGCCAGCCAAAGTGTCACT |
| | ACATCTAGGTATAGTTATATGCACTGGTACCAACAGAAACCAGGACAGC |
| | CACCCAAACTCCTCATCATGTATGCATCCAACCTAGAATCTGGGGTCCCT |
| | GCCAGGTTCAGTGGCAGTGGGTCTGGGACAGACTTCACCCTCAACATCCA |
| | TCCTGTGGAGGAGGAGGATATTGCAATATATTACTGTCAGCACAGTTGGG |
| | AGATWCCGTGGACGTTCGGTGGAGGCACCAAGCTGGAAATCAAACGGG |
| | CTGATGCTGCACCAACTGTATCCATCTTCCCACCATCCAGTGAGCAGTTA |
| | ACATCTGGAGGTGCCTCAGTCGTGTGCTTCTTGAACAACTTCTACCCCAG |
| | AGACATCAATGTCAAGTGGAAGATTGATGGCAGTGAACGACAAAATGGT |
| | GTCCTGAACAGTTGGACTGATCAGGACAGCAAAGACAGCACCTACAGCA |
| | TGAGCAGCACCCTCACATTGACCAAGGACGAGTATGAACGACATAACAG |
| | CTATACCTGTGAGGCCACTCACAAGACATCAACTTCACCCATCGTCAAGA |
| | GCTTCAACA |
| 1H1 | ATGGAGACAGACACACTCCTGCTATGGGTGCTGCTGCYCTGGGTTCCAGG |
| (SEQ ID NO: 6) | TTCCACTGGTGACATTGTGCTGACACAGTCTCCTGCTTCCTTACCTGTATC |
| | TCTGGGGCAGAGGGCCACCATTTCATGTAGGGCCAGCCAAAGTGTCACT |
| | ACATCTAGGTATAGTTATATGCACTGGTACCAACAGAAACCAGGACAGC |
| | CACCCAAACTCCTCATCATGTATGCATCCAACCTAGAATCTGGGGTCCCT |
| | GCCAGGTTCAGTGGCAGTGGGTCTGGGACAGACTTCACCCTCAACATCCA |
| | TCCTGTGGAGGAGGAGGATATTGCAATATATTACTGTCAGCACAGTTGGG |
| | AGATTCCGTGGACGTTCGGTGGAGGCACCAAGCTGGAAATCAAACGGGC |
| | TGATGCTGCACCAACTGTATCCATCTTCCCACCATCCAGTGAGCAGTTAA |
| | CATCTGGAGGTGCCTCAGTCGTGTGCTTCTTGAACAACTTCTACCCCAGA |
| | GACATCAATGTCAAGTGGAAGATTGA |
| 1G1 | ATGGAGACAGACACACTCCTGCTATGGGTGCTGCTGCTCTGGGTTCCAGG |
| (SEQ ID NO: 7) | TTCCACTGGTGACATTGTGCTGACACAGTCTCCTGCTTCCTTACCTGTATC |
| | TCTGGGGCAGAGGGCCACCATTTCATGTAGGGCCAGCCAAAGTGTCACT |
| | ACATCTAGGTATAGTTATATGCACTGGTACCAACAGAAACCAGGACAGC |
| | CACCCAAACTCCTCATCATGTATGCATCCAACCTAGAATCTGGGGTCCCT |
| | GCCAGGTTCAGTGGCAGTGGGTCTGGGACAGACTTCACCCTCAACATCCA |
| | TCCTGTGGAGGAGGAGGATATTGCAATATATTACTGTCAGCACAGTTGGG |
| | AGATTCCGTGGACGTTCGGTGGAGGCACCAAGCTGGAAATCAAACGGGC |
| | TGATGCTGCACCAACTGTATCCATCTTCCCACCATCCAGTGAGCAGTTAA |
| | CATCTGGAGGTGCCTCAGTCGTGTGCTTCTTGAACAACTTCTACCCCAGA |
| | GACATCAATGTCAAGTGGAAGATTGATGGCAGTGAACGACAAAATGGTG |
| | TCCTGAACAGTTGGACTGATCAGGACAGCAAAGACAGCACCTAC |
| 1G10 | ATGAGTGTGCCCACTCAGGTCCTGGGGTTGCTGCTGCTGTGGCTTACAGA |
| (SEQ ID NO: 8) | TGCCAGATGTGACATCCAGATGACTCAGTCTCCAGCCTCCCTATCTGTAT |
| | CTGTGGGAGAAACTGTCACCATCACATGTCGAGCAAGTGAGAATATTTA |
| | CAATAATTTAGCATGGTATCAGCAGAAACAGGGAAAATCTCCTCAGCTC |
| | CTGGTCTATGCTGCAACAAACTTAGCAGATGGTGTGCCATCAAGGTTCAG |
| | TGGCAGTGGATCAGGCACACAGTTTTCCCTCAAGATCAACAGCCTGCAGT |
| | CTGAAGATTTTGGGAGTTATTACTGTCAACATTTTTGGGGTATTCCGTAC |
| | ACGTTCGGAGGGGGGACCAAGCTGGAAATAACACGGGCTGATGCTGCAC |
| | CAACTGTATCCATCTTCCCACCATCCAGTGAGCAGTTAACATCTGGAGGT |
| | GCCTCAGTCGTGTGCTTCTTGAACAACTTCTACCCCAGAGACATCAATGT |
| | CAAGTGGAAGATTGATGG |
| 2G7 | ATGGTTTTCACACCTCAGATACTTGGACTTATGCTTTTTTGGATTTCAGCC |
| (SEQ ID NO: 9) | TCCAGAGGTGATATTATACTAACTCAGTCTCCAGCCACCCTGTCTGTGAC |
| | TCCAGGAGATAGCGTCAGTCTTTCCTGCAGGGCCAGCCAGAGTATTAGCA |
| | ACAACCTACACTGGTATCAACAAAAATCACATGAGTCTCCAAGGCTTCTC |
| | ATCAAGTATGCTTCCCAGTCCATCTCTGGGATCCCCTCCAGGTTCAGTGG |
| | CAGTGGATCAGGGACAGATTTCACTCTCAGTATCAACAGTGTGGAGACT |
| | GAAGATTTTGGAATGTATTTCTGTCAACAGAGTAACAGCTGGCCGCTCAC |
| | GTTCGGTGCTGGGACCAAGCTGGAGCTGAAACGGGCTGATGCTGCACCA |
| | ACTGTATCCATCTTCCCACCATCCAGTGAGCAGTTAACATCTGGAGGTGC |
| | CTCAGTCGTGTGCTTCTTGAACAACTTCTACCCCAAAGACATCAATGTCA |
| | AGTGGAAGATTGATGGCAGTGAACGACAAAATGGCGTCCTGAACAGTTG |
| | GACTGATC |
| 1D12 | ATGGAGACAGACACAATCCTGCTATGGGTGCTGCTGCTCTGGGTTCCAGG |
| (SEQ ID NO: | CTCCACTGGTGACATTGTGCTGACCCAATCTCCAGCTTCTTTGGCTGTGTC |
| 10) | TCTAGGGCAGAGGGCCACCATCTCCTGCAAGGCCAGCCAAAGTGTTGAT |
| | TATGATGGTGATAGTTATATGAACTGGTACCAACAGAAACCAGGACAGC |
| | CACCCAAACTCCTCATCTATGCTGCATCCAATCTAGAATCTGGGATCCCA |
| | GCCAGGTTTAGTGGCAGTGGGTCTGGGACAGACTTCACCCTCAACATCCA |
| | TCCTGTGGAGGAGGAGGATGCTGCAACCTATTACTGTCAGCAAAGTAAT |
| | GAGGATCCTCCGTTCGGAGGGGGGACCAAGCTGGAAATAAAACGGGCTG |
| | ATGCTGCACCAACTGTATCCATCTTCCCACCATCCAGTGAGCAGTTAACA |
| | TCTGGAGGTGCCTCAGTCGTGTGCTTCTTGAACAACTTCTACCCCAAAGA |
| | CATCAATGTCAAGTGGAAGATTGATGGCAGTGAACGACAAAATGGCGTC |
| | CTGAACAGTTGGACTGATCAGGACAGCAAAGACAGCACCTACAGCATGA |
| | GCAGCACCCTCACGTTGACCAAGGACGAGTATGAACGACATAA |

TABLE 2

| Antibodies | |
|---|---|
| Against SEMA- | |
| 4A | Heavy Chain |
| 2C1 | ATGAATTGCAGCTGGATTATCTTCTTCCTGATGGCAGTGGTTACAGGGGT |
| (SEQ ID NO: | CAATTCAGAGGTTCAGCTGCACCAGTCTGGGGCAGACCTTGTGAAGCCAG |
| 11) | GGGCCTCAGTCAAGTTGTCCTGCACAGCTTCTGGCTTCCACATTAAAGAC |
| | ACCTATATACACTGGATGAAACAGAGGCCTGAACAGGGCCTGGAGTGGA |
| | TTGGAAGGATTGATCCTGCGAATGGTAATTCTGAATATGACCCGAAGTTC |
| | CAGGGCAAGGCCACTATAACAGCAGACACATCCTCCAACACAGCCTACC |
| | TGCACCTCAGCAGCCTGACATCTGAGGACACTGCCGTCTATTACTGTGCT |
| | GAATCCAATTACTTCGGTAGTAGTCCCTATGCTTTGGACTACTGGGGTCA |
| | AGGAACCTCAGTCACCGTCTCCTCAGCCAAAACAACAGCCCCATCGGTCT |
| | ATCCACTGGCCCCTGTGTGTGGAGATACAACTGGCTCCTCGGTGACTCTA |
| | GGATGCCTGGTCAAGGGTTATTTCCCTGAGCCAGTG |
| 1A3 | ATGGCTGTTCTGGTGCTGTTCCTCTGCCTGGTTGCATTTCCAAGCTGTGTC |
| (SEQ ID NO: | CTGTCCCAGGTGCAGCTGAAGGAGTCAGGACCTGTCCTGGTGGCGCCCTC |
| 12) | ACAGAGCCTGTCCATCACTTGCACTGTCTCTGGGTTTTCATTAACCAGCTA |
| | TGCTATACACTGGGTTCGCCAGCCTCCAGGAAAGGGTCTGGAGTGGCTGG |
| | GAGTAATCTGGGCTGGTGGAAGCACAGATTATAATTCAGCTCTCATGTCC |
| | AGACTAAACATCAACAAAGACAACTCCAAGAGCCAAGTTTTCTTAAAAA |
| | TGAACAGTCTGCAAATTGATGACACAGCCATGTACTACTGTGCCAGACAC |
| | TCATCAAAAGCCTACTATAGTAACTACTTTGACTACTGGGGCCAAGGCAC |
| | CACTCTCACAGTCTCCTCAGCCAAAACAACAGCCCCATCGGTCTATCCAC |
| | TGGCCCCTGTGTGTGGAGATACAAGTGGC |
| 1E7 | ATGAACTTCGGGCTCAGCTTGATTTTCCTTGTCCTTGTTTTAAAAGGTGTC |
| (SEQ ID NO: | CAGTGTGAAGTGAAGCTGGTGGAGTCTGGGGGAGGCTTAGTGAAGCCTG |
| 13) | GAGGGTCCCTGAATCTCTCCTGTGCAGCCTCTGGATTCACTTTCAATAGCT |
| | TTACCATGTCTTGGGTTCGCCAGACTCCGGCGAAGAGGCTGGAGTGGGTC |
| | GCAACCATTAGTAGTAGTGGTTATAACACCTACTATCCAGACAGTGTGAA |
| | GGGCCGATTCACCATCTCCAGAGACAATGCCAGGAACACCCTGTACCTGC |
| | AAATGAGCAGTCTGAGGTCTGAGGACACGGCCATGTATTACTGTGCAAG |
| | ACATGGGAAGATCTATGATGGTTACCCATTTGCTATGGACTTCTGGGGTC |
| | AAGGAACCTCAGTCACCGTCTCCTCAGCCAAAACAACAGCCCCATCGGTC |
| | TATCCACTGGCCCCTGTGTGTGGAGATACAAGTGGCTCC |
| 1E2 | ATGGGATGGAGCTGTATCATCCTCTTTTTGGTAGCAGCAGCTACAGGTGT |
| (SEQ ID NO: | CCACTCCCAGGTCCAACTGCAGCAGCCTGGGGCTGAGCTGGTGAAGCCTG |
| 14) | GGGCTTCAGTGAAGCTGTCCTGCAAGGCTTCTGGCTACACCTTCACCAGC |
| | TACTGGATGCACTGGGTGAAGCAGAGGCCTGGACAGGGCCTTGAATGGA |
| | TTGGTAATATTGACCCTTCTGATAGTGAAACTCATTACAATCAAAAGTTC |
| | AAGGACAAGGCCACATTGACTGTAGACAAATCTTCCAGCACAGCCTACA |
| | TGCAACTCAGCAGCCTGACATCTGAGGACTCTGCGGTCTATTACTGTGCA |
| | AGACACTACGATGGCTATGCTGTGGACTACTGGGGTCAAGGAACCTCAGT |
| | CACCGTCTCCTCAGCCAAAACAACAGCCCCATCGGTCTATCCACTGGCCC |
| | CTGTGTGTGGAGATACAAGTGGCTCCTCGGTGACTCTAGGATGCCTGGTC |
| | AAGGGTTATTTCCCTGAGCCAGTGACCTTGACCTGGAACTCTGGATCCCT |
| | GTCCAGTGGTGTGCACACCTTCCCAGCTGTCCTGCAGTCTGACC |
| 1H1 | ATGGGATGGAGCTGTATCATCCTCTTTTTGGTAGCAGCAGCTACAGGTGT |
| (SEQ ID NO: | CCACTCCCAGGTCCAACTGCAGCAGCCTGGGGCTGAGCTGGTGAAGCCTG |
| 15) | GGGCTTCAGTGAAGCTGTCCTGCAAGGCTTCTGGCTACACCTTCACCAGC |
| | TACTGGATGCACTGGGTGAAGCAGAGGCCTGGACAGGGCCTTGAATGGA |
| | TTGGTAATATTGACCCTTCTGATAGTGAAACTCATTACAATCAAAAGTTC |
| | AAGGACAAGGCCACATTGACTGTAGACAAATCTTCCAGCACAGCCTACA |
| | TGCAACTCAGCAGCCTGACATCTGAGGACTCTGCGGTCTATTACTGTGCA |
| | AGACACTACGATGGCTATGCTGTGGACTACTGGGGTCAAGGAACCTCAGT |
| | CACCGTCTCCTCAGCCAAAACAACAGCCCCATCGGTCTATCCACTGGCCC |
| | CTGTGTGTGGAGATACAAGTGGCTCCTCGGTGACTCTAGGATGCCTGGTC |
| | AAGGGTTATTTCCCTGAGCCAGTGACCTTGACCTGGAACTCTGGAT |
| 1G1 | ATGGGATGGAGCTGTATCATCCTCTTTTTGGTAGCAGCAGCTACAGGTGT |
| (SEQ ID NO: | CCACTCCCAGGTCCAACTGCAGCAGCCTGGGGCTGAGCTGGTGAAGCCTG |
| 16) | GGGCTTCAGTGAAGCTGTCCTGCAAGGCTTCTGGCTACACCTTCACCAGC |
| | TACTGGATGCACTGGGTGAAGCAGAGGCCTGGACAGGGCCTTGAATGGA |
| | TTGGTAATATTGACCCTTCTGATAGTGAAACTCATTACAATCAAAAGTTC |
| | AAGGACAAGGCCACATTGACTGTAGACAAATCTTCCAGCACAGCCTACA |
| | TGCAACTCAGCAGCCTGACATCTGAGGACTCTGCGGTCTATTACTGTGCA |
| | AGACACTACGATGGCTATGCTGTGGACTACTGGGGTCAAGGAACCTCAGT |
| | CACCGTCTCCTCAGCCAAAACAACAGCCCCATCGGTCTATCCACTGGCCC |
| | CTGTGTGTGGAGATACAAGTGGCTCCTCGGTGACTCTAGGATGCCTGGTC |
| | AAGGGTTATTTCCCTGAGCCAGTGACCTTGACCTGGAACTCTGGATCCCT |
| | GTCCAGTGGTGTGCACACCTTCCCAGCTGTCCTG |
| 1G10 | ATGAACTTCGGGCTCAGCTTGATTTTCCTTGTCCTTGTTTTAAAAGGTGTC |
| (SEQ ID NO: | CAGTGTGAAGTGAAACTGGTGGAGTCTGGGGGAGGCTTAGTGAAGCCTG |
| 17) | GAGGGTCCCTGAAACTCTCCTGTGCAGCCTCTGGATTCACTTTCAGTGGC |
| | TATACCATGTCTTGGGTTCGCCAGACTCCGGCGAAGAGGCTGGAGTGGGT |
| | CGCAACCATTAGTGGTTCTGGTGGTAATATTTACTATCCAGACACTGTGA |
| | AGGGCCGATTCACCATCTCCAGAGACAATGCCAGGAACACCCTGTACCTG |
| | CAAATGAGCAGTCTGAGGTCTGAGGACACGGCCATGTATTACTGTACAA |
| | GACAGGAGGGAGGCTTACGCGGCTTTGCTTACTGGGGCCAAGGGACTCT |
| | GGTCACTGTCTCTGCAGCCAAAACGACACCCCCATCTGTCTATCCACTGG |
| | C |
| 2G7 | ATGGAAAGGCACTGGATCTTTCTACTCCTGTTGTCAGTAACTGCAGGTGT |
| (SEQ ID NO: | CCACTCCCAGGTCCAGCTGCAGCAGTCTGGGGCTGAACTGGCAAGACCTG |
| 18) | GGGCCTCAGTGAAGATGTCCTGCAAGGCTTCTGGCTACACCTTTATTACC |
| | TACACGATGCACTGGGTAAAACAGAGGCCTGGACAGGGTCTGGAATGGA |
| | TTGGATACATTAATCCTAGCAGTGATTATACTAATTACAATCAGAGATTC |
| | AAGGACAAGGCCACATTGACTGCAGACAAATCCTCCAGCACAGCCTACA |
| | TGCAACTGAACAGCCTGACATCTGAGGACTCTGCAGTCTATTACTGTGCA |
| | AGGGGGGCTCCGGCTTACTGGGGCCAAGGGACTCTGGTCACTGTCTCTGC |
| | AGCCAAAACAACAGCCCCATCGGTCTATCCACTGGCCCCTGTGTGTGGAG |
| | ATACAACTGGCTCCTCGGTGACTCTAGGATGCCTGGTCAAGGGTTATTTC |
| | CCTGAGCCAGTGACCTTGACCTGGAACTCTGGATCCCTGTCCAGTGGTGT |
| | GCACACCTTCCCAGC |
| 1D12 | ATGAACAGGCTTACTTCCTCATTGCTGCTGCTGATTGTCCCTGCATATGTC |
| (SEQ ID NO: | CTGTCCCAGGTTACTCTGAAAGAGTCTGGCCCTGGGATATTGCAGCCCTC |
| 19) | CCAGACCCTCAGTCTGACTTGTTCTTTCTCTGGGTTTTCACTGAGCACTTC |
| | TGGTATGGGTGTGAGCTGGATTCGTCAGCCTTCAGGAAAGGGTCTGGAGT |
| | GGCTGGCACACATTTACTGGGATGATGACAAGCGCTATAACCCATCCCTG |
| | AAGAGCCGGCTCACAATCTCCAAGGATACCTCCAGCAACCAGGTATTCCT |
| | CAAGATCACCAGTGTGGACACTGCAGATACTGCCACATACTACTGTGCTC |
| | GTACTATGATTACGACGAGGAACTACTTTGACTACTGGGGCCAAGGCACC |
| | ACTCTCACAGTCTCCTCAGCCAAAACAACAGCCCCATCGGTCTATCCACT |
| | GGCCCCTGTGTGTGGAGATACAACTGGCTCCTCGGTGACTCTAGGATGCC |
| | TGGTCAAGGGTT |

In one embodiment, the antigen-binding molecule may comprise an immunoglobulin heavy chain domain (HC) (as shown in Table 2) and an immunoglobulin light chain domain (LC) (as shown in Table 1).

In an embodiment, the HC comprises an amino acid sequence having at least 80% sequence identity to SEQ ID NO: 11-19. In an embodiment, the HC comprises an amino acid sequence having at least 85% sequence identity to SEQ ID NO: 11-19. In an embodiment, the HC comprises an amino acid sequence having at least 90% sequence identity to SEQ ID NO: 11-19. In an embodiment, the HC comprises an amino acid sequence having at least 91% sequence identity to SEQ ID NO: 11-19. In an embodiment, the HC comprises an amino acid sequence having at least 92% sequence identity to SEQ ID NO: 11-19. In an embodiment, the HC comprises an amino acid sequence having at least 93% sequence identity to SEQ ID NO: 11-19. In an embodiment, the HC comprises an amino acid sequence having at least 94% sequence identity to SEQ ID NO: 11-19. In an embodiment, the HC comprises an amino acid sequence having at least 95% sequence identity to SEQ ID NO: 11-19. In an embodiment, the HC comprises an amino acid sequence having at least 96% sequence identity to SEQ ID NO: 11-19. In an embodiment, the HC comprises an amino acid sequence having at least 97% sequence identity to SEQ ID NO: 11-19. In an embodiment, the HC comprises an amino acid sequence having at least 98% sequence identity to SEQ ID NO: 11-19. In an embodiment, the HC comprises an amino acid sequence having at least 99% sequence identity to SEQ ID NO: 11-19.

In an embodiment, the LC comprises an amino acid sequence having at least 80% sequence identity to SEQ ID NO: 1-10. In an embodiment, the LC comprises an amino acid sequence having at least 85% sequence identity to SEQ ID NO: 1-10. In an embodiment, the LC comprises an amino acid sequence having at least 90% sequence identity SEQ ID NO: 1-10. In an embodiment, the LC comprises an amino acid sequence having at least 91% sequence identity to SEQ ID NO: 1-10. In an embodiment, the LC comprises an amino acid sequence having at least 92% sequence identity to SEQ ID NO: 1-10. In an embodiment, the LC comprises an amino acid sequence having at least 93% sequence identity to SEQ ID NO: 1-10. In an embodiment, the LC comprises an amino acid sequence having at least 94% sequence identity to SEQ ID NO: 1-10. In an embodiment, the LC comprises an amino acid sequence having at least 95% sequence identity to SEQ ID NO: 1-10. In an embodiment, the LC comprises an amino acid sequence having at least 96% sequence identity to SEQ ID NO: 1-10. In an embodiment, the LC comprises an amino acid sequence having at least 97% sequence identity to SEQ ID NO: 1-10. In an embodiment, the LC comprises an amino acid sequence having at least 98% sequence identity to SEQ ID NO: 1-10. In an embodiment, the LC comprises an amino acid sequence having at least 99% sequence identity to SEQ ID NO: 1-10.

In an embodiment, the antigen-binding molecule is an antibody or a SEMA-4A binding fragment thereof. In an embodiment, the SEMA-4A-binding fragment is selected from the group consisting of a Fab fragment, a scFab, and Fab′, a single chain variable fragment (scFv) and a one-armed antibody. In an embodiment, the antigen-binding molecule is a humanized antibody or SEMA-4A-binding fragment thereof.

In an illustrative aspect, an isolated nucleic acid molecule is provided. The isolated nucleic acid comprises a nucleic acid sequence encoding any of the antigen-binding molecules described herein.

In an illustrative aspect, an expression construct is provided. The expression construct comprises a nucleic acid sequence encoding the antigen-binding molecule of any of the antigen-binding molecules described herein, operably linked to one or more regulatory sequences. In an illustrative aspect, a host cell comprising the expression construct is provided.

In an illustrative aspect, antigen-binding molecules that specifically bind to FCRL3 are provided.

In at least one embodiment, antibodies generated against FCRL3 can be used to generate an antibody single-chain variable fragment that can then be used to prepare a chimeric antigen receptor (CAR). The antibody single-chain variable fragment is a chimeric protein made up of light (LC) and heavy (HC) chains of immunoglobins, connected with a short linker peptide. In accordance with the present disclosure, the antibodies are selected in advance for their binding ability to the target antigen, FCRL3. In some embodiments, the linker between the variable regions may consist of hydrophilic residues with stretches of glycine and serine in it for flexibility as well as stretches of glutamate and lysine for added solubility.

TABLE 3

| Antibodies | |
|---|---|
| FCRL3 | Light Chain |
| 2F1 | ATGGAATCACAGACTCAGGTCCTCATGTCCCTGCTGCTCTGGGTATCTGG |
| (SEQ ID NO: | TACCTGTGGGGACATTGTTATGACACAGTCTCCATCCTCCCTGAGTGTGT |
| 20) | CAGCAGGACATAAGGTCACTATGAGCTGCAAGTCCAGTCAGAGTCTATT |
| | AAACAGTAGAAATCAAAAGAACTACTTGGCCTGGTACCAGCAGAAACCA |
| | TGGCAGCCTCCTAAACTTCTGATCTACGGGGCATCCACTAGGGAATCTGG |
| | GGTCCCTGATCGCTTCACAGGCAGTGGATCTGGAACAGATTTCACTCTCA |
| | CCATCAGCAGTGTGCAGGCTGAAGACCTGGCAGTTTATTACTGTCAGAAT |
| | GATTATAGTTATCCTCCCACGTTCGGTGCTGGGACCAAGCTGGAGCTGAA |
| | ACGGGCTGATGCTGCACCAACTGTATCCATCTTCCCACCATCCAGTGAGC |
| | AGTTAACATCT |
| 2H3 | ATGGATTTTCAAGTGCAGATTTTCAGCTTCCTGCTAATCAGTGCTTCAGTC |
| (SEQ ID NO: | ATAATGTCCAGAGGACAAATTGTTCTCTCCCAGTCTCCAGCAATCCTGTC |
| 21) | TGCATCTCCAGGGGAGAAGGTCACAATGACTTGCAGGGCCAGCTCAAGT |
| | GTAAGTTACATGCACTGGTACCAGCAGAAGCCAGGATCCTCCCCCAAAC |
| | CCTGGATTTATGCCACATCCAACCTGGCTTCTGGAGTCCCTGCTCGCTTCA |
| | GTGGCAGTGGGTCTGGGACCTCTTACTCTCTCACAATCAGCAGAGTGGAG |
| | GCTGAAGATGCTGCCACTTATTACTGCCAGCAGTGGAGTAGTAACCCACC |
| | GACGTTCGGTGGAGGCACCAAGCTGGAAATCAAACGGGCTGATGCTGCA |
| | CCAACTGTATCCATCTTCCCACCATCCAGTGAGCAGTTAACATCTGGAGG |
| | TGCCTCAGTCGTGTGCTTCTTGAACAACTTCTACCCCAAAGACATCAATG |
| | TCAAGTG |
| 2C9 | ATGAAGTTGCCTGTTAGGCTGTTGGTGCTGATGTTCTGGATTCCTGGTTCC |
| (SEQ ID NO: | AGCAGTGATGTTGTGATGACCCAAACTCCACTCTCCCTGCCTGTCAGTCT |
| 22) | TGGAGATCAAGCCTCCATCTCTTGCAGATCTAGTCAGAGCCTTGTATACA |
| | GTAATGGAAACACCTATTTACATTGGTACCTGCAGAAGCCAGGCCAGTCT |
| | CCAAAGCTCCTGATCTACAAAGTTTCCAACCGATTTTCTGGGGTCCCAGA |
| | CAGGTTCAGTGGCAGTGGATCAGGGACAGATTTCACACTCAAGATCAGC |
| | AGAGTGGAGGCTGAGGATCTGGGAGTTTATTTCTGCTCTCAAAGTACACA |
| | TGTTCCATTCACGTTCGGCTCGGGGACAAAGTTGGAAATAAAACGGGCT |
| | GATGCTGCACCAACTGTATCCATCTTCCCACCATCCAGTGAGCAGTTAAC |
| | ATCTGGAGGTGCCTCAGTCGTGTGCTTCTTGAACAACTTCTACCCCAAAG |
| | ACATCAATGTCAAGTGGAA |
| 1C4 | ATGGAGACAGTCACACTCCTGCTATGGGTGCTACTGCTCTGGGTTCCAGG |
| (SEQ ID NO: | CTCCACTGGTGACATTGTGCTGACCCAATCTCCAGCTTCTTTGGCTGTGTC |
| 23) | TCTAGGGCAGAGGGCCACCATCTCCTGCAGAGCCAGCGAAAGTGTCAGT |
| | ATTCGTGGTACTAGTTTAATGCACTGGTACCAACAGAAACCAGGATATCC |
| | ACCCAAACTCCTCATCTATGCTGCATCCAACCTAGAATCTGGGGTTCCTG |
| | CCAGGTTTAGTGGCAGAAGGTCTGGGACAGACTTCACCCTCAACATTCAT |
| | CCTGTGGAGGAAGATGATGCTGCAACCTATTACTGTCAGCAAAGTAGGG |
| | AATATCCGCTCACGTTCGGTGCTGGGACCAAGCTGGAGCTGAAACGGGC |
| | TGATGCTGCACCAACTGTATCCATCTTCCCACCATCCAGTGAGCAGTTAA |
| | CATCTG |
| 1A7 | ATGATGTCCTCTGCTCAGTTCCTTGGTCTCCTGTTGCTCTGTTTTCAAGGT |
| (SEQ ID NO: | ACCAGATGTGATATCCAGATGACACAGACTACATCCTCCCTGTCTGCCTC |
| 24) | TCTGGGAGACAGAGTCACCATCAGTTGCAGTGCAAGTCAGGGCATTAAC |
| | AATTATTTAAACTGGTATCAGCAGAAACCAGATGGAACTGTTAAACTCCT |
| | GATCTATTACACATCAAGTTTACACTCAGGAGTCCCATCAAGGTTCAGTG |
| | GCAGTGGGTCTGGGACAGATTATTCTCTCACCATCAGCAACCTGGAACCT |
| | GAAGATATTGCCACTTACTATTGTCAGCAGTATAGTAAGCTTCCATTCAC |
| | GTTCGGCTCGGGGACAAAGTTGGAAATAAAACGGGCTGATGCTGCACCA |
| | ACTGTATCCATCTTCCCACCATCCAGTGAGCAGTTAACATCTGGAGGTGC |
| | CTCAGTCGTGTGCTTCTTGAACAACTTCTACCCCAAAGACATCAATGTCA |
| | AGTGGAAGATTGATGGCAGTGAACGACAAAATGGCGTCCTGAACAGTTG |
| | GACTGAT |
| 1A1 | ATGGAATCACAGACTCAGGTCCTCATGTCCCTGCTGCTCTGGGTATCTGG |
| (SEQ ID NO: | TACCTGTGGGGACATTGTGATGACACAGTCTCCATCCTCCCTGAGTGTGT |
| 25) | CAGCAGGAGATAAGGTCACTATGAGCTGCAAGTCCAGTCAGAGTCTGTT |
| | AAACAGTAGAAACCAAAAGAACTACTTGGCCTGGTACCAGCAGAAACCA |
| | TGGCAGCCTCCTAAACTGCTGATCTACGGGGCATCCACTAGGGAATCTGG |
| | GGTCCCTGATCGCTTCACAGGCAGTGGATCTGGAACAGATTTCACTCTCA |
| | CCATCAGCAGTGTGCAGGCTGAAGACCTGGCAGTTTATTACTGTCAGAAT |
| | GATTATAGTTATCCTCCGACGTTCGGTGGAGGCACCAAGCTGGAAATCAA |
| | ACGGGCTGATGCTGCACCAACTGTATCCATCTTCCCACCATCCAGTGAGC |
| | AGTTAACATCTGGAGGTGCCTCAGTCGTGTGCTTCTTGAACAACTTCTAC |
| | CCCAAAGACATCAATGTCAAGTGGAAGATTGATGGCAGTGAACGACAAA |
| | ATGGCGTCCTGAACAGTTGGACTGATCAGGACAGCAAAGACAGCACCTA |
| | CA |
| 1E10 | ATGGATTTTCAAGTGCAGATTTTCAGCTTCCTGCTAATCAGTGCTTCAGTC |
| (SEQ ID NO: | AAAATGTCCAGAGGACAAATTGTTCTCTCCCAGTCTCCAGCAATCCTGTC |
| 26) | TGCATCTCCAGGGGAGAAGGTCACAATGACTTGCAGGGCCAGCTCAAGT |
| | GTAAGTTACATGCACTGGTACCAGCAGAAGCCAGGATCCTCCCCCAAAC |
| | CCTGGATTTATGCCACATCCAACCTGGCTTCTGGAGTCCCTGCTCGCTTCA |
| | GTGGCAGTGGGTCTGGGACCTCTTACTCTCTCACAATCAGCAGAGTGGAG |
| | GCTGAAGATGCTGCCACTTATTACTGCCAGCAGTGGAGTAGTAACCCACC |
| | GACGTTCGGTGGAGGCACCAAGCTGGAAATCAAACGGGCTGATGCTGCA |
| | CCAACTGTATCCATCTTCCCACCATCCAGTGAGCAGTTAACA |
| 2D4 | ATGGAGTCACAGACTCAGGTCTTTGTATACATGTTGCTGTGGTTGTCTGG |
| (SEQ ID NO: | TGTTGATGGAGACATTGTGATGACCCAGTCTCAAAAATTCATGTCCACAT |
| 27) | CAGTAGGAGACAGGGTCAGCGTCACCTGCAAGGCCAGTCAGAATGTGGG |
| | TACTAATGTAGCCTGGTATCAACAGAAACCAGGGCAATCTCCTAAAGCA |
| | CTGATTTACTCGGCATCCTACCGGTACAGTGGAGTCCCTGATCGCTTCAC |
| | AGGCAGTGGATCTGGGACAGATTTCACTCTCACCATCAGCAATGTGCAGT |
| | CTGAAGACTTGGCAGAGTATTTCTGTCAGCAATATAACAGCTATCCGTAC |
| | ACGTTCGGAGGGGGGACCAAGCTGGAAATAAAACGGGCTGATGCTGCAC |
| | CAACTGTATCCATCTTCCCACCATCCAGTGAGCAGTTAACATCTGGAGGT |
| | GCCTCAGTCGTGTGCTTCTTGAACAACTTCTACCCCAAAGAC |
| 2B3 | ATGTCCTCTGCTCAGTTCCTTGGTCTCCTGTTGCTCTGTTTTCAAGGTACC |
| (SEQ ID NO: | AGATGTGATATCCAGATGACACAGACTACATCCTCCCTGTCTGCCTCTCT |
| 28) | GGGAGACAGAGTCACCATCAGTTGCAGGGCAAGTCAGGATATTAGCAAT |
| | TATTTAAACTGGTATCAGCAGAAACCAGATGGAACTGTTAAACTCCTGAT |
| | CTACTACACATCAAGATTACACTCAGGAGTCCCATCAAGGTTCAGTGGCA |
| | GTGGGTCTGGGACAGATTATTCTCTCACCATCAGCAACCTGGAACCTGAA |
| | GATATTGCCACTTACTATTGTCAGCAGTATAGTAAGCTTCCGCTCACGTT |
| | CGGTGCTGGGACCAAGCTGGAGCTGAAACGGGCTGATGCTGCACCAACT |
| | GTATCCATCTTCCCACCATCCAGTGAGCAGTTAACATCTGGAGGTGCCTC |
| | AGTCGTGTGCTTCTTGAACAACTTCTACCCCAAAGACATCAATGTCAAGT |
| | GGAAGATTGATGGCAGTGAACGACAAAATGGCGTCCTGAACAGTTGGAC |
| | TGATCAGGACAGCAAAGACAGCACCTACAGCATGA |
| 1E12 | ATGGAGACAGACACACTCCTGCTATGGGTGCTGCTGCTCTGGGTTCCAGG |
| (SEQ ID NO: | TTCCACTGGTGACATTGTGCTGACACAGTCTCCTGCTTCCTTAGCTGTATC |
| 29) | TCTATGGCAGAGGGCCACCATCTCATGCAGGGCCAGCCAAAGTGTCAGT |
| | ACATCTAGGTATAGTTATATGTACTGGTACCAACAGAAACCAGGACAGC |
| | CACCCAAACTCCTCATCAAGTATGCATCCAACCTAGAATCTGGGGTCCCT |
| | GCCAGGTTCAGTGGCAGTGGGTCTGGGACAGACTTCACCCTCAACATCCA |
| | TCCTGTGGAGGAGGAGGATACTGCAACATATTACTGTCAGCACAGTTGG |
| | GAGATTCCGTACACGTTCGGAGGGGGGACCATGCTGGAAATAAAACGGG |
| | CTGATGCTGCACCAACTGTATCCATCTTCCCACCATCCAGTGAGCAGTTA |
| | ACATCTGGAGGTGCCTCAGTCGTGTGCTTCTTGAACAACTTCTACCCCAA |
| | AGACATCAATGTCAAGTGGAAGATTGATGGCAGTGAACGACAAAATGGC |
| | GTCCTGAACAGTTGGACTGATCAGGACAGCAAAGACAGCACCTACAGCA |
| | TGAGCAGCACCCTCACGTTGACCAAGGACGAGTATGAACGACATAACAG |
| | CTATACCTGTGAG |
| 1H5 | ATGGAGACAGACACACTCCTGCTATGGGTGCTGCTGCTCTGGGTTCCAGG |
| (SEQ ID NO: | TTCCACAGGTAACATTGTGCTGACCCAATCTCCAGCTTCTTTGGCTGTGTC |
| 30) | TCTAGGGCAGAGGGCCACCATATCCTGCAGAGCCAGTGAAAGTGTTGAT |
| | AGTTATGGCAATAGTTTTATGCACTGGTACCAACAGAAACCAGGACAGC |
| | CACCCAAACTCCTCATCTATCTTGCATCCAACCTAGAATCTGGGGTCCCT |
| | GCCAGGTTCAGTGGCAGTGGGTCTAGGACAGACTTCACCCTCACCATTGA |
| | TCCTGTGGAGGCTGATGATGCTGCAACCTATTACTGTCAACAAAATAATG |
| | AGGATCCGTGGACGTTCGGTGGAGGCACCAAGCTGGAAATCAAACGGGC |
| | TGATGCTGCACCAACTGTATCCATCTTCCCACCATCCAGTGAGCAGTTAA |
| | CATCTGGAGGTGCCTCAGTCGTGTGCTTCTTGAACAACTTCTACCCCAAA |
| | GACATCAATGTCAAGTGGAAGATTGATGGCAGTGAACGACAAAATGGCG |
| | TCCTGAACAGTTGGACTGATCAGGACAGCAAAGACAGCACCTACAGCAT |
| | GAGCAGCACCCTCACGTTGACCAAGGACGAGTATGAACGACATAACAGC |
| | TATACCTGTGAGGCCACTCACAA |

TABLE 4

| Antibodies | |
|---|---|
| Against FCRL3 | Heavy Chain |
| 2F1 | ATGGGATGGAGCTGGATCTTTCTCTTTCTCCTGTCAGGAACTGCAGGTGT |
| (SEQ ID NO: | CCTCTCTGAGGTCCAGTTGCAACAATCTGGACCTGAGCTGGTGAAGCCTG |
| 31) | GGGCTTCAGTGAAGATATCCTGTAAGACTTCTGGATACACTTTCACTGAC |
| | TACTACATGAACTGGGTGAAGCAGAGCCATGGAAAGAGCCTTGAGTGGA |
| | TTGGAGATGTTAATCCTAACAATAATAATACTAGATACAATCAGAAGTTC |
| | AAGGGCAAGGCCACATTGACCGTAGACAAGTCCTCCAGCACAGCCTACA |
| | TGGAGCTCCGCAGCCTGACATCTGAGGACTCTGCAGTCTATTTCTGTGCA |
| | AGGGGGGGGCTGTTCTATGCTATGGACAACTGGGGTCAAGGAACCTCAG |
| | TCACCGTCTCCTCAGCCAAAACAACACCCCCATCAGTCTATCCACTGGCC |
| | CCTGGGTGTGGAGATACAACTGGTTCCTCCGTGACTCTGGGATGCCTGGT |
| | CAAGGGCTACTTCCCTGAGTCAGTGACTGTGACTTGGAACTCTGGATCCC |
| | TGTCCAGCAGTGTGCACACCTTCCCAGCTCTCCTGCAGTCTGGACTCT |
| 2C9 | ATGGGATGGAGCTGGATCTTTCTCATTCTCCTGTCAGGAACTGCAGGTGT |
| (SEQ ID NO: | CCTCTCTGAGGTCCAGCTGCAACAATCTGGACCTGAGCTGGTGAAGCCTG |
| 32) | GGGCTTCAGTGAAGATATCCTGTAAGGCTTCTGGATACACGTTCAAAGAC |
| | TACTACATGAACTGGGTGAAGCAGAGCCATGGAAAGAGCCTTCAGTGGA |
| | TTGGAGATATTAATCCTAACAATGGTGGTACTACCTACAACCAGAAGTTC |
| | AAGGGCAAGGCCACATTGACTGTAGACAAGTCCTCCAGCATAGCCTACA |
| | TGGAGCTCCGCAGCCTGACATCTGAGGACTCTGCAGTCTATTACTGTGCA |
| | AGCGACTTTGACTACTGGGGCCAAGGCACCACTCTCACAGTCTCCTCAGC |
| | CAAAACGACACCCCCATCTGTCTATCCACTGGCCCCTGGATCTGCTGCCC |
| | AAACTAACTCCATGGTGACCCTGGGATGCCTGGTCAAGGGCTATTTCCCT |
| | GAGCCAGTGACAGTGACCTGGAACTCTGGATCCCTGTCCAGCGGTGTGCA |
| | CACCTTCCCAGCTGTCCTGCA |
| 1C4 | ATGGCTGTCCTGGCACTGCTCCTCTGCCTGGTGACATTCCCAAGCTGTGTC |
| (SEQ ID NO: | CTGTCCCAGGTGCAGCTGAAGGAGTCAGGACCTGGCCTGGTGGCGCCCTC |
| 33) | ACAGAGCCTGTCCATCACATGCACTGTCTCAGGGTTCTCATTAACCAGCT |
| | ATGGTGTAAGCTGGGTTCGCCAGCCTCCAGGAAAGGGTCTGGAGTGGCT |
| | GGGAGTAATATGGGGTGACGGGAGCACAAATTATCATTCAGCTCTCATAT |
| | CCAGACTGAGCATCAGCAAGGATAACTCCAAGAGCCAAGTTTTCTTAAA |
| | ACTGAACAGTCTGCAAACTGATGACACAGCCACGTACTACTGTGCCAAA |
| | GGGGGGGACGGCTTTGACTACTGGGGCCAAGGCACCACTCTCACAGTCTC |
| | CTCAGCCAAAACAACAGCCCCATCGGTCTATCCACTGGCCCCTGTGTGTG |
| | GAGATACAAGTGGCTCCTCGGTGACTCTAGGATGCCTGGTCAAGGGTTAT |
| | TTCCCTGAGCCAGTGACCTTGACCTGGAACTCT |
| 1A7 | ATGGGATGGAGCTGGATCTTTCTCCTCTTCCTGTCAGGAACTGCAGGTGT |
| (SEQ ID NO: | CCTCTCTGAGGTCCACCTGCAACAGTCTGGACCTGAGCTGGTGAAGCCTG |
| 34) | GGGCTTCAGTGAAGATACCCTGCAAGGCTTCTGGATACACATTCACTGAC |
| | TACAACATGGACTGGGTGAAGCAGAGCCATGGAAAGAGCCTTGAGTGGA |
| | TTGGAGATATTAATCCTAACAATGGTGGTGCTATCTACAACCAGAAGTTC |
| | AAGGGCAAGGCCACATTGACTGTCGACAAGTCCTCCAACACAGCCTACA |
| | TGGAGCTCCGCAGCCTGACATCTGAGGACACTGCAGTCTATTACTGTGCA |
| | AGAAGGGATTACCAAAGTCCCTTTGACTACTGGGGCCAAGGCACCACTCT |
| | CACAGTCTCCTCAGCCAAAACAACACCCCCATCAGTCTATCCACTGGCCC |
| | CTGGGTGTGGAGATACAACTGGTTCCTCCGTGACTCTGGGATGCCTGGTC |
| | A |
| 1A1 | ATGGGATGGAGCTGGATCTTTCTCTTTCTCCTGTCAGGAACTGCAGGTGT |
| (SEQ ID NO: | CCTCTCTGAGGTCCAGCTGCAACAATCTGGACCTGAGCTGGTGAAGCCTG |
| 35) | GGACTTCAATGAAGATATCCTGTAAGGCTTCTGCATACACGTTCACTGAC |
| | TACTTCATGAACTGGGTGAAGCAGAGCCATGGAAAGAGCCTTGAGTGGA |
| | TTGGAGATATTAATCCTAACAATGGTGCTACTAGGTACAATCAGAGGTTC |
| | AAGGTCAAGGCCACACTGACTGTAGACAAGTCCTCCAGCACAGCCTACA |
| | TGGAGCTCCGCAGCCTGACATCTGAGGACTCTGCAGTCTATTACTGTGCA |
| | AGAGGGGACGAAGGGTTTGTTTACTGGGGCCAAGGGACTCTGGTCACTG |
| | TCTCTGCAGCCAAAACGACACCCCCATCTGTCTATCCACTGGCCCCTGGA |
| | TCTGCTGCCCAAACTAACTCCATGGTGACCCTGGGATGCCTGGTCAAGGG |
| | CTATTTCCCTGAGCCAGTGACAGTGACCTGGAACTCTGGATCCCTGTCCA |
| | GCGGTGTGCACACCTTCCCAGCTGTCCTGCAGTCTGACCTCTACACTC |
| 1E10 | ATGGCTGTCTTGGGGCTGCTCTTCTGCCTGGTGACATTCCCAAGCTGTGTC |
| (SEQ ID NO: | CTATCCCAGGTGCAGCTGAAGCAGTCAGGACCTGGCCTAGTGCAGCCCTC |
| 36) | ACAGAGCCTGTCCATCACCTGCACAGTCTCTAGTTTCTCATTAACTAGCTA |
| | TGGTGTACACTGGGTTCGCCAGTCTCCAGGAAAGGGTCTGGAGTGGCTGG |
| | GAGTGATATGGAGTGGTGTAAGCACAGACTATAATGCAGCTTTCATATCC |
| | AGACTGAACATCAGCAAGGACAATTCCAAGAGCCAAGTTTTCTTTAAAAT |
| | GAACAGTCTGCAAACTGATGACACAGCCATATATTACTGTGCCAGAAATG |
| | GGCTCGGGCTACGTAGGGCCTCCATGGACTACTGGGGTCAAGGAACCTC |
| | AGTCACCGTCTCCTCAGCCAAAACAACAGCCCCATCGGTCTATCCACTGG |
| | CCCCTGTGTGTGGAGATACAAGTGGCTCCTCGGTGACTCTAGGATGCCTG |
| | GTCAAGGGTTATTTCCCTGAGCCAGTGACCTTGACCTGGAACTCTGGATC |
| | CCTGTCCA |
| 2D4 | ATGGAATGGATCTGGATCTTTCTCTTCATCCTGTCAGGAACTGCAGGTGT |
| (SEQ ID NO: | CCAATCCCAGGTTCAGCTGCAGCAGTCTGGAGCTGAGCTGGCGAGGCCTG |
| 37) | GGGCTTCAGTGAAGCTGTCCTGCAAGGCTTCTGGCTACACCTTCACAAGC |
| | TATGGTATAAGCTGGGTGAAGCAGAGAACTGGACAGGGCCTTGAGTGGA |
| | TTGGAGAGATTTATCCTAGAAGTGGTAATACTTACTACAATGAGAAGTTC |
| | AAGGGCAAGGCCACACTGACTGCAGACAAATCCTCCAGCACAGCGTACA |
| | TGGAGCTCCGCAGCCTGACATCTGAGGACTCTGCGGTCTATTTCTGTGCA |
| | AGATCGGACTACTGGGGCCAAGGCACCACTCTCACAGTCTCCTCAGCCAA |
| | AACAACAGCCCCATCGGTCTATCCACTGGCCCCTGTGTGTGGAGATACAA |
| | GTGGCTCCTCGGTGACTCTAGGATGCCTGGTCAAGGGTTATTTCCCTGAG |
| | CCAGTGACCTTGACCTGGAACTCTGGATCCCTGTCCAGTGGTGTGCACAC |
| | CTTCCCAGCTGTCCTGCAGTCTGACCTCTACA |
| 2B3 | ATGAACTTGGGGCTCAGCTTGATTTTCCTTGTCCTTGTTTTAAAAGGTGTC |
| (SEQ ID NO: | CAGTGTGAAGTGAAGCTGGTGGAGTCTGGGGGAGGCTTAGTGCAGCCTG |
| 38) | GAGGGTCCCTGAAACTCTCCTGTGCAGCCTCTGGATTCACTTTCAGTGAC |
| | TATTACATGTATTGGGTTCGCCAGACTCCAGAGAAGAGGCTGGAGTGGGT |
| | CGCATACATTAGTAATGGTGGTGGTAGCACCTATTATCCAGACACTGTAA |
| | AGGGCCGATTCACCATCTCCAGAGACAATGCCAAGAACACCCTGTACCTG |
| | CAAATGAGCCGTCTGAAGTCTGAGGACACAGCCATGTATTACTGTGCAAG |
| | ACTAGCGGAGGATGGTTACTACGGGGACTACTTTGACTACTGGGGCCAA |
| | GGCACCACTCTCACAGTCTCCTCAGCCAAAACGACACCCCCATCTGTCTA |
| | TCCACTGGCCCCTGGATCTGCTGCCCAAACTAACTCCATGGTGACCCTGG |
| | GATGCCTGGTCAAGGGCTATTTCC |
| 1E12 | ATGGGATGGAGCTGTATCATTGTCCTCTTGGTATCAACAGCTACAGGTGT |
| (SEQ ID NO: | CCACTCCCAGGTCCAACTGCAGCAGCCTGGGTCTGAACTGGTGAGGCCTG |
| 39) | GGACTTCAGTGAAGTTGTCCTGCAAGGCTTCTGGCTATATTTTCACCAGCT |
| | ACTGGATGCACTGGGTAAAGCAGAGGCCTGGACAAGGCCTTGAGTGGAT |
| | CGGAGTGATTGATCCTTCTGATACTTATACTAACTACAATCAAAAGTTCA |
| | AGGGCAAGGCCACATTGACTGTAGACACATCCTCCAGCACAGCCTACAT |
| | GCAGCTCAGCAGCCTGACATCTGAGGACTCTGCGGTCTATTACTGTGCAA |
| | GAGAGGAGTATGGTGACTACGTAGGAGGCTTTGACTTCTGGGGCCAAGG |
| | CACCACTCTCACAGTCTCCTCAGCCAAAACAACACCCCCATCAGTCTATC |
| | CACTGGCCCCTGGGTGTGGAGATACAACTGGTTCCTCCGTGACTCTGGGA |
| | TGCCTGGTCAAGGGCTACTTCCCTGAGTCAGTGAC |
| 1H5 | ATGGAATGGAGCTGGGTCTTTCTCTTCCTCCTGTCAGTAATTGCAGGTGTC |
| (SEQ ID NO: | CAATCCCAGGTTCAACTGCAGCAGTCTGGGGCTGAACTGGTGAGGCCTGG |
| 40) | GGCTTCAGTGACGCTGTCCTGCAAGGCTTCGGGCTACACATTTTCTGACT |
| | ATGAAATGCACTGGGTGAAGCAGACACCTGTGCATGGCCTGGAATGGAT |
| | TGGAGCTTTTGATCCTGAAACTGGTACTACTGCCTACAATCAGAAGTTCA |
| | AGGACAAGGCCATACTGATTGCAGACAAATCCTCCAGCACAGCCTACAT |
| | GGAGCTCCGCAGCCTGACATCTGAGGACTCTGCCGTCTATTACTGTACAA |
| | GAGCCTACTATAATCACTACGTTCACTTTGACTACTGGGGCCAAGGCACC |
| | ACTCTCACAGTCTCCTCAGCCAAAACAACACCCCCATCAGTCTATCCACT |
| | GGCCCCTGGGTGTGGAGATACAACTGGTTCCTCCGTGACTCTGGGATGCC |
| | TGGTCAAGGGCTACTTCCCTGAGTCAGTGACTGTGAC |

In one embodiment, the antigen-binding molecule may comprise an immunoglobulin heavy chain domain (HC) (as shown in Table 4) and an immunoglobulin light chain domain (LC) (as shown in Table 3).

In an embodiment, the HC comprises an amino acid sequence having at least 80% sequence identity to SEQ ID NO: 31-40. In an embodiment, the HC comprises an amino acid sequence having at least 85% sequence identity to SEQ ID NO: 31-40. In an embodiment, the HC comprises an amino acid sequence having at least 90% sequence identity to SEQ ID NO: 31-40. In an embodiment, the HC comprises an amino acid sequence having at least 91% sequence identity to SEQ ID NO: 31-40. In an embodiment, the HC comprises an amino acid sequence having at least 92% sequence identity to SEQ ID NO: 31-40. In an embodiment, the HC comprises an amino acid sequence having at least 93% sequence identity to SEQ ID NO: 31-40. In an embodiment, the HC comprises an amino acid sequence having at least 94% sequence identity to SEQ ID NO: 31-40. In an embodiment, the HC comprises an amino acid sequence having at least 95% sequence identity to SEQ ID NO: 31-40. In an embodiment, the HC comprises an amino acid sequence having at least 96% sequence identity to SEQ ID NO: 31-40. In an embodiment, the HC comprises an amino acid sequence having at least 97% sequence identity to SEQ ID NO: 31-40. In an embodiment, the HC comprises an amino acid sequence having at least 98% sequence identity to SEQ ID NO: 31-40. In an embodiment, the HC comprises an amino acid sequence having at least 99% sequence identity to SEQ ID NO: 31-40.

In an embodiment, the LC comprises an amino acid sequence having at least 80% sequence identity to SEQ ID NO: 20-30. In an embodiment, the LC comprises an amino acid sequence having at least 85% sequence identity to SEQ ID NO: 20-30. In an embodiment, the LC comprises an amino acid sequence having at least 90% sequence identity SEQ ID NO: 20-30. In an embodiment, the LC comprises an amino acid sequence having at least 91% sequence identity to SEQ ID NO: 20-30. In an embodiment, the LC comprises an amino acid sequence having at least 92% sequence identity to SEQ ID NO: 20-30. In an embodiment, the LC comprises an amino acid sequence having at least 93% sequence identity to SEQ ID NO: 20-30. In an embodiment, the LC comprises an amino acid sequence having at least 94% sequence identity to SEQ ID NO: 20-30. In an embodiment, the LC comprises an amino acid sequence having at least 95% sequence identity to SEQ ID NO: 20-30. In an embodiment, the LC comprises an amino acid sequence having at least 96% sequence identity to SEQ ID NO: 20-30. In an embodiment, the LC comprises an amino acid sequence having at least 97% sequence identity to SEQ ID NO: 20-30. In an embodiment, the LC comprises an amino acid sequence having at least 98% sequence identity to SEQ ID NO: 20-30. In an embodiment, the LC comprises an amino acid sequence having at least 99% sequence identity to SEQ ID NO: 20-30.

In an embodiment, the antigen-binding molecule is an antibody or a FCRL3 binding fragment thereof. In an embodiment, the FCRL3-binding fragment is selected from the group consisting of a Fab fragment, a scFab, a Fab′, a single chain variable fragment (scFv) and a one-armed antibody. In an embodiment, the antigen-binding molecule is a humanized antibody or a FCRL3-binding fragment thereof.

In an embodiment, the disclosure is directed to an isolated nucleic acid comprising a nucleic acid sequence encoding any of the antigen-binding molecules described herein.

In an embodiment, the disclosure is directed to an expression construct comprising a nucleic acid sequence encoding the antigen-binding molecule of any of the antigen-binding molecules described herein, operably linked to one or more regulatory sequences. In an embodiment, the disclosure is directed to a host cell comprising the expression construct.

As described above, the antigen-binding molecules described above that specifically bind to a specific antigen (FCRL3 or SEMA-4A) can have different binding regions. Tables 5-10 list the amino acid sequences of the antigen binding molecules described including the amino acid sequence of the framework region (FR) and complementarity determining region (CDR) that binds specifically to, or interacts specifically with, the target antigen. Tables 5-7 list the amino acid sequences of the heavy chains and Tables 8-10 list the amino acid sequences of the light chains.

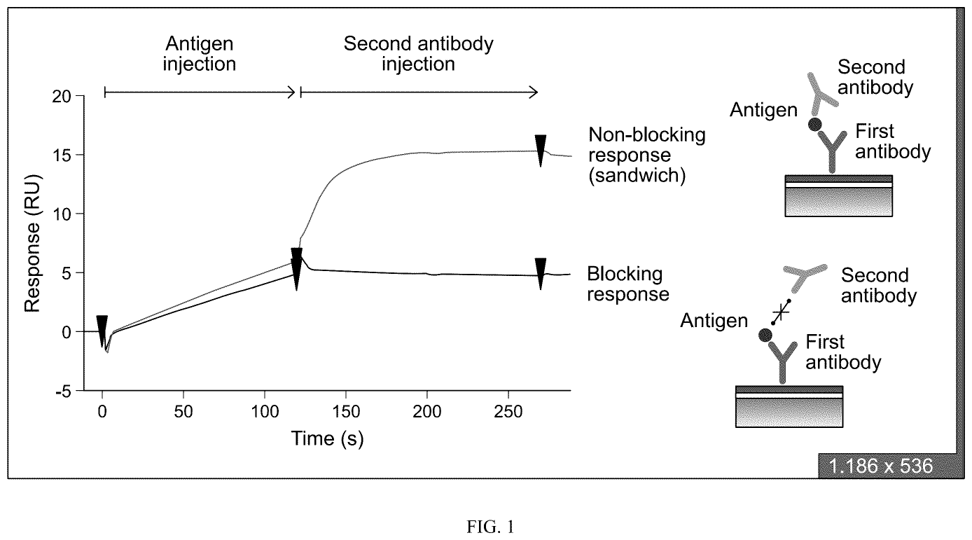

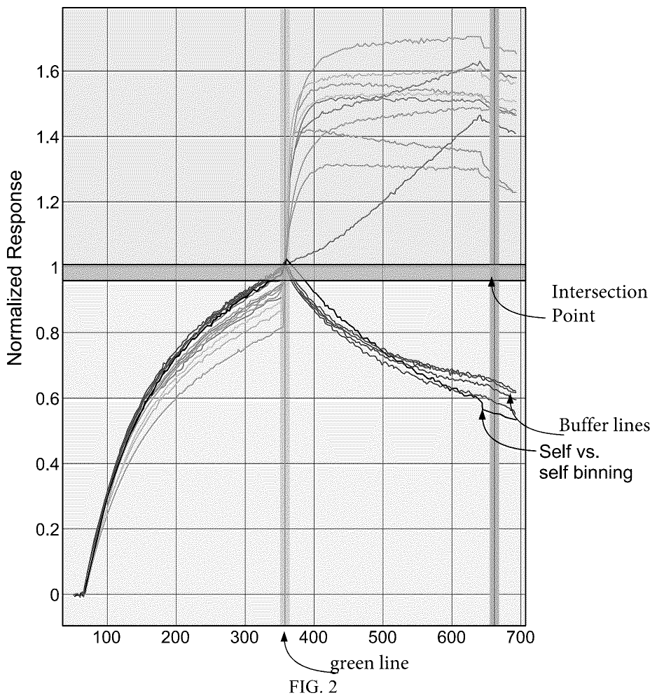

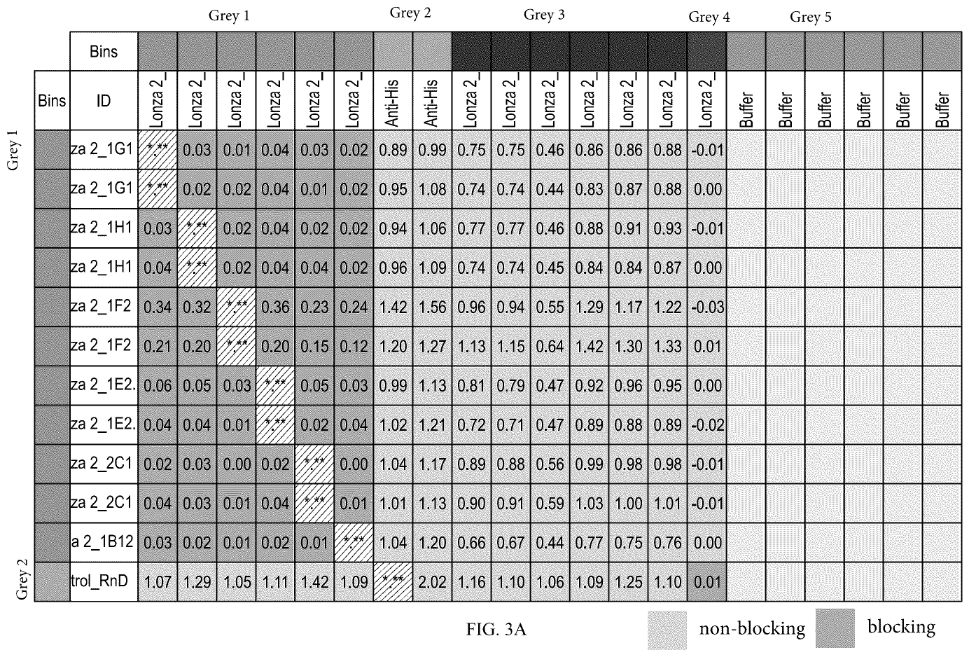

FIGS. 1 and 2 illustrate binning experiments where single antigen-binding molecules that specifically bind to an antigen are chemically conjugated to a chip and other antigen-binding molecules that specifically bind to the same antigen are individually added in solution. As shown in FIG. 2, the sensor gram itself represents a single antigen-binding molecule (e.g., antibody) that has been chemically conjugated to the chip. Each individual trace represents an antigen that has been added to the chip (to the left of the green line) and which is very quickly changed for a particular antigen-binding molecule (e.g., antibody) in solution to the right of the green line. If both, the chip conjugated and in solution, antigen-binding molecules (e.g., antibody) can bind the antigen at the same time the trace goes up to reflect the increase in protein bound that is bound to the chip. The binding of this additional antigen-binding molecule (e.g., antibody) may also cross-link the antigen making it less prone to falling off. If both, the chip conjugated and in solution, antigen-binding molecule (e.g., antibody) cannot bind to the antigen at the same time, the assay shows the antigen falling off when the in solution antigen-binding molecule (e.g., antibody) is added. When the same antigen-binding molecule (e.g., antibody) is in solution and is bound to the chip, the in solution antigen-binding molecule (e.g., antibody) has no place to bind the antigen and the trace decreases. FIG. 2 illustrates this self-vs self-binning, the buffer lines, and the intersection point or cut of point.

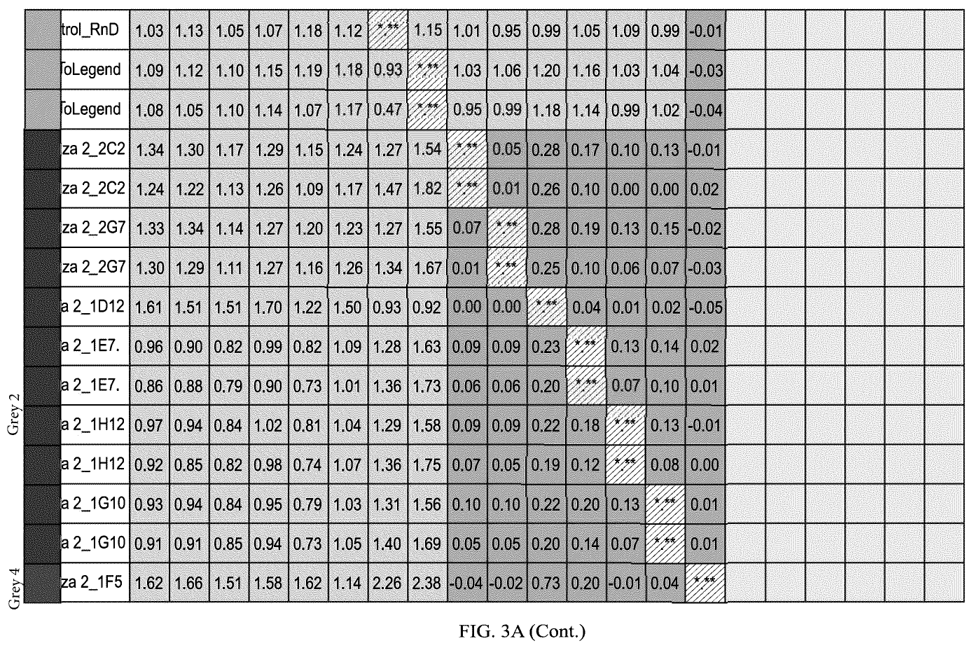

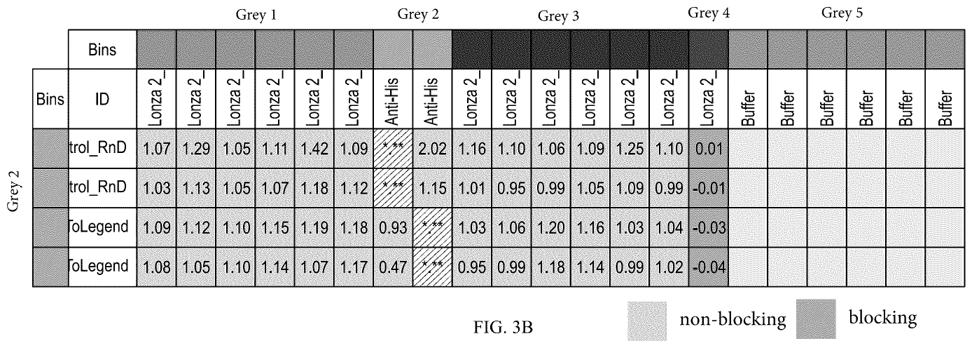

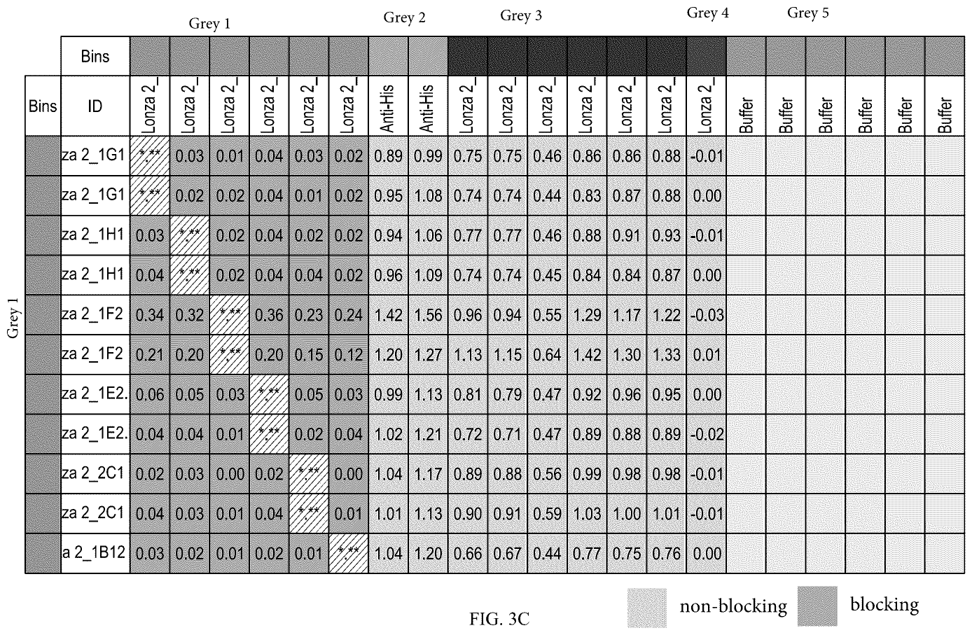

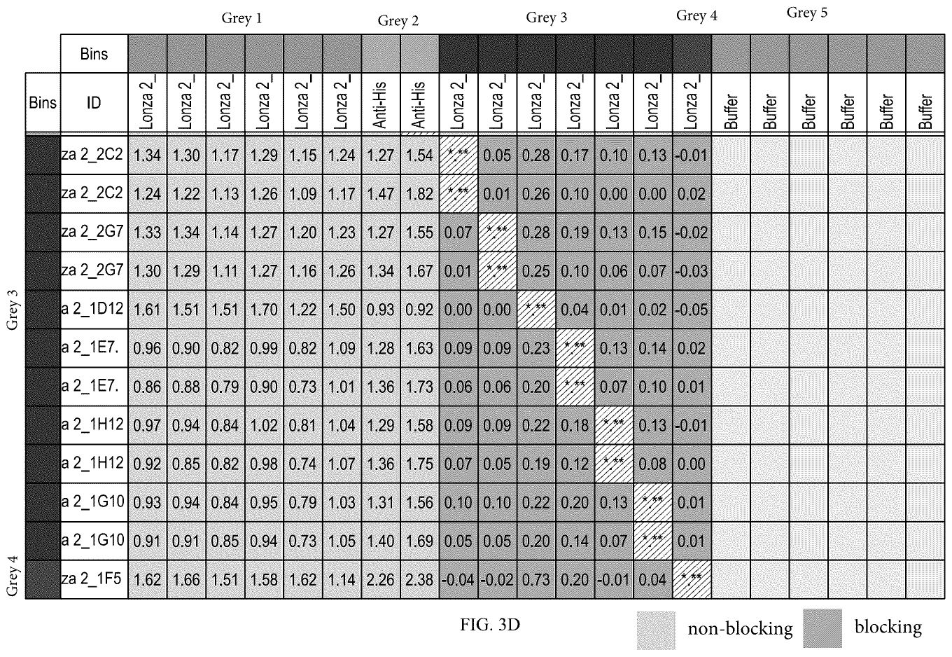

FIG. 3A-3D illustrates a binning experiment for antibodies and controls targeting SEMA-4A. FIG. 3A-3D illustrate blocking and non-blocking squares. FIG. 3A illustrates a binning experiment for antibodies targeting SEMA-4A. As shown in FIG. 3B, the Anti-His antibodies are positive controls. All the antibodies block themselves. The His antibodies may not block themselves because of complex formation. As shown, there can be two bins of SEMA-4A antibodies grey 1 bin (FIG. 3B) and grey 3 (FIG. 3C). All the grey 1 bin antibodies block each other, bind to the same region of SEMA-4A, and do not block the grey 3 bin antibodies. The grey 3 bin antibodies may bind to different regions of the SEMA-4A than the grey 1 bin antibodies. As shown in FIG. 3D, 1F5 may bind to the antigen if it is first to bind the antigen but may no longer be able to bind to SEMA-4A if members of the grey 1 bin bind to SEMA-4A first. Thus, the binding order of the antibodies to SEMA-4A may be important.

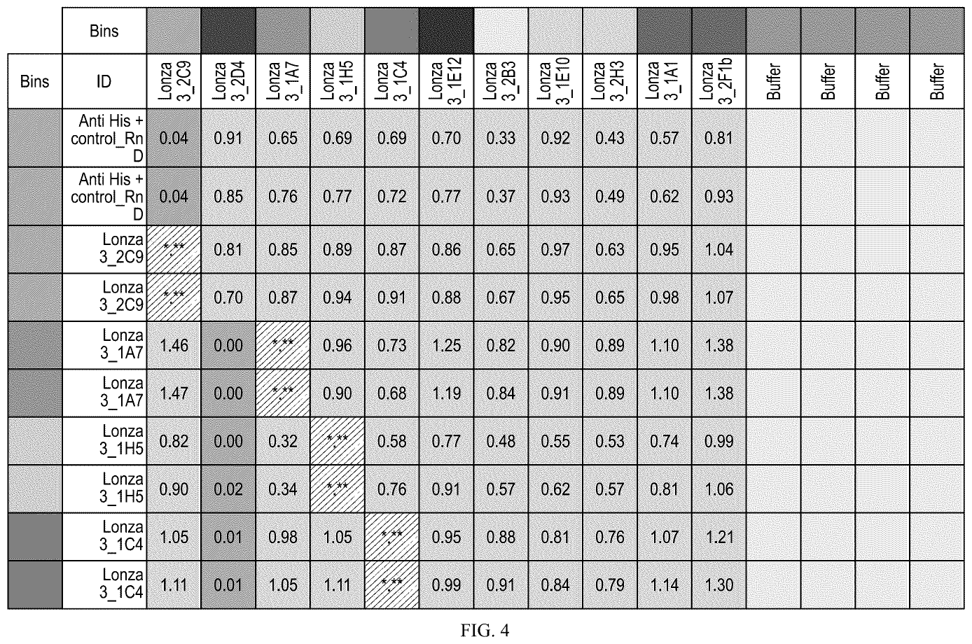

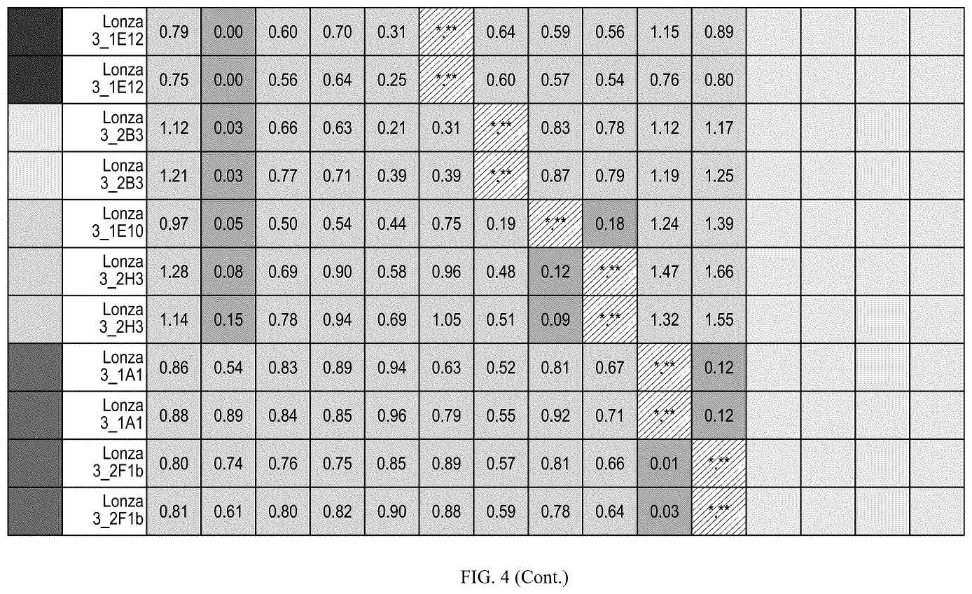

FIG. 4 illustrates a binning experiment for antibodies targeting FCRL3 illustrating blocking and non-blocking squares. As shown, the antibodies represented different bins with the exception of 1E10 and 2H3 that bin together, 1A1, and 2F1 that bin together. Further, many antibodies in the separate bins appear to block 2D4. This may be because the binding of the antibodies from various bins has an allosteric effect and results in a conformational change in FCRL3 that prevents 2D4 from binding.

### Example 1: Generation of Monoclonal Antibodies

Novel purified monoclonal antibodies recognizing SEMA4A through the immunization of 12 female mice of three different strains (Balb/c CD1, F1 cross) were generated by infusion of two million NIH/3T3 cells overexpressing SEMA4A antigen and of recombinant peptides covering the extracellular domain (ECD) of SEMA4A. Ten days following the sixth boost, serum was tested by bead and cell-based ranking using an iQue high throughput flow-cytometer (Intellicyte). The six best mice were given a final boost of 1M cells and 5 ug of protein. Three days later, mice were euthanatized and splenocytes were fused 1:1 with a myeloma fusion partner (FOX-NY). Obtained hybridomas were screened using simultaneous high-throughput against three MM cell lines (KMS11, H929, U266) and a bead-conjugated SEMA4A ECD antigen. Up to fifteen hybridoma clones were selected and subcloned by a ClonePix® 2 Colony Picker in semi-solid methylcellulose selection media containing fluorescently labeled secondary antibodies to detect cells producing mouse IgG. These cells were screened with cells and protein on beads. 10-fold difference in MFI between SEMA4A+ HEK293 cells and parental HEK293 was considered optimal. The positive cells from this cloning were re-arrayed into eight subclones and frozen. Variable domain sequencing was performed on each antibody. Novel purified monoclonal antibodies recognizing FCRL3 were similarly generated.

### Example 2: Flow Cytometry

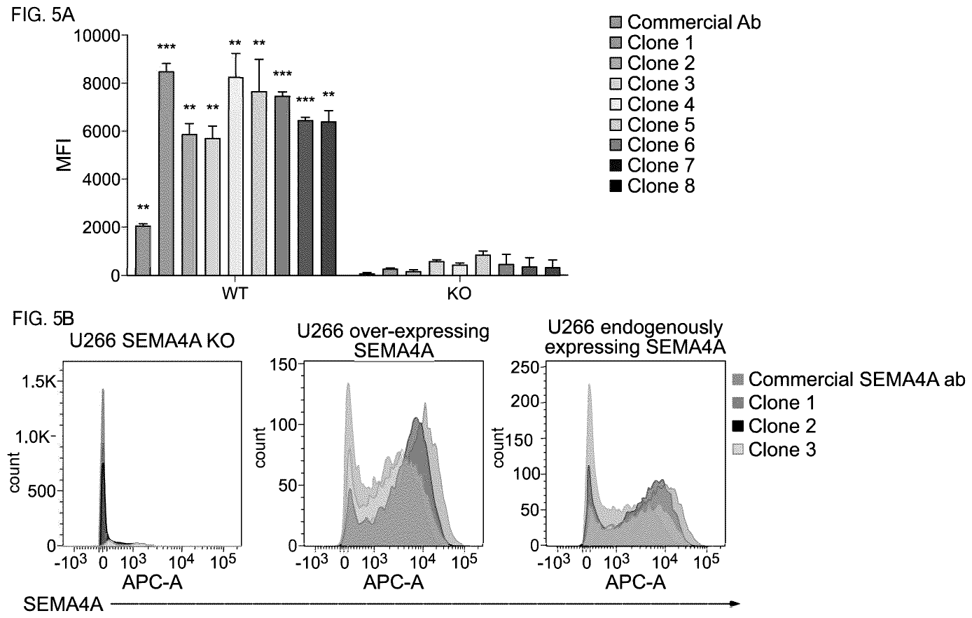

Different concentrations (0.1 ug, 0.2 ug, 0.4 ug and 0.8 ug) of antibodies were tested in flow cytometry on two MM cell lines (U266 and KMS11) endogenously expressing the target (SEMA-4A) at different levels. Cells virally infected to overexpress or genetically inactivate SEMA4A (KO) and the commercial antibody (Biolegend cat #148406) validate specific binding were also tested. As shown in FIGS. 5A-B, eight out of eleven binders are able to specifically recognize SEMA4A on MM cells. FIG. 5A shows the MFI values from flow-cytometric analyses of eight selected antibodies conjugated with secondary antibodies or the SEMA4A commercially available antibody (Cat #148406 Biolegend) in U266 MM cell lines endogenously expressing SEMA4A (WT) or knocked-out for SEMA4A (KO). Experiments were performed in duplicates and results on MM cells compared to KO cells (p<0.05). FIG. 5B shows the representative FACS plots with three antibodies with different binding strength. Highest for clone 1 and lowest for clone 3. Clone 2 shows intermediate affinity.

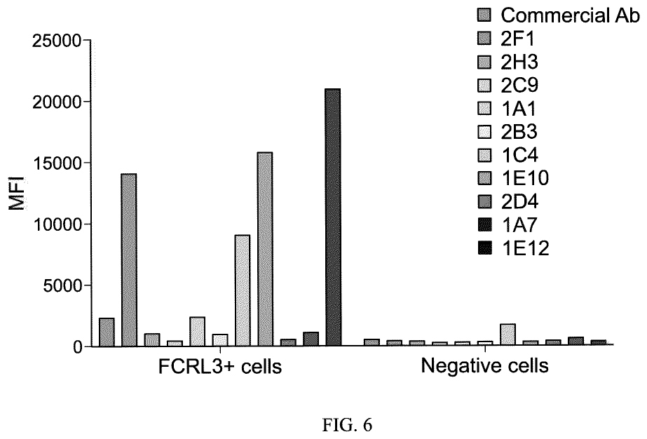

Different concentrations (0.1 ug, 0.2 ug, 0.4 ug and 0.8 ug) of antibodies were tested in flow cytometry on cell lines (U266 and DAUDI) endogenously expressing the target (FCRL3) at different levels. Cells virally infected to overexpress or genetically inactivate FCRL3 and the commercial antibody (Rndsystems—FAB3126P) validate specific binding were also tested. FIG. 6 shows the shows the MFI values from flow-cytometric analyses of antibodies targeting FCRL3.

TABLE 5

| Name | Chain | | FR1 | | CDR1 | | FR2 | | CDR2 |
|---|---|---|---|---|---|---|---|---|---|
| 1F5 | Heavy | SEQ. | EVQLQQSGPELVKS | SEQ. | GYTFTDY | SEQ | MDWVKQSHGK | SEQ. | IYPNNG |
| | | NO. 41 | GDLVKMSCKAS | NO. 65 | Y | NO. 89 | SLEWIGY | NO. 113 | GT |
| 2F1 | Heavy | SEQ. | EVQLQQSGPELVKP | SEQ. | GYTFTDY | SEQ | MNWVKQSHGK | SEQ | VNPNNN |
| | | NO. 42 | GASVKISCKTS | NO. 66 | Y | NO. 90 | SLEWIGD | NO. 114 | NT |
| 1A7 | Heavy | SEQ. | EVHLQQSGPELVKP | SEQ. | GYTFTDY | SEQ. | MDWVKQSHGK | SEQ. | INPNNG |
| | | NO. 43 | GASVKIPCKAS | NO. 67 | N | NO. 91 | SLEWIGD | NO. 115 | GA |
| 1H5 | Heavy | SEQ. | QVQLQQSGAEL VR | SEQ. | GYTFSDY | SEQ. | MHWVKQTPVH | SEQ. | FDPETG |
| | | NO. 44 | PGASVTLSCKAS | NO. 68 | E | NO. 92 | GLEWIGA | NO. 116 | TT |
| 1B12 | Heavy | SEQ. | QVQLQQSGAELAK | SEQ. | GYTFSSY | SEQ | MHWVKQRPGQ | SEQ. | IDPSSSY |
| | | NO. 45 | PGASVKMSCKAS | NO. 69 | W | NO. 93 | DLEWIGY | NO. 117 | A |
| 1E2 | Heavy | SEQ. | QVQLQQPGAELVK | SEQ. | GYTFTSY | SEQ. | MHWVKQRPGQ | SEQ. | IDPSDSE |
| | | NO. 46 | PGASVKLSCKAS | NO. 70 | W | NO. 94 | GLEWIGN | NO. 118 | T |
| 1A3 | Heavy | SEQ. | QVQLKESGPVLVAP | SEQ. | GFSLTSY | SEQ. | IHWVRQPPGKG | SEQ. | IWAGGS |
| | | NO. 47 | SQSLSITCTVS | NO. 71 | A | NO. 95 | LEWLGV | NO. 119 | T |
| 1D12 | Heavy | SEQ. | QVTLKESGPGILQPS | SEQ. | GFSLSTS | SEQ. | VSWIRQPSGKG | SEQ. | IYWDDD |
| | | NO. 48 | QTLSLTCSFS | NO. 72 | GMG | NO. 96 | LEWLAH | NO. 120 | K |
| 1E7 | Heavy | SEQ. | EVKLVESGGGLVKP | SEQ. | GFTFNSF | SEQ. | MSWVRQTPAK | SEQ. | ISSSGYN |
| | | NO. 49 | GGSLNLSCAAS | NO. 73 | T | NO. 97 | RLEWVAT | NO. 121 | T |
| 1H1 | Heavy | SEQ. | QVQLQQPGAELVK | SEQ. | GYTFTSY | SEQ. | MHWVKQRPGQ | SEQ. | IDPSDSE |
| | | NO. 50 | PGASVKLSCKAS | NO. 74 | W | NO. 98 | GLEWIGN | NO. 122 | T |
| 2C2 | Heavy | SEQ. | EVQLHQSGADLVK | SEQ. | GFHIKDT | SEQ | IHWMKQRPEQG | SEQ. | IDPANG |
| | | NO. 51 | PGASVKLSCTAS | NO. 75 | Y | NO. 99 | LEWIGR | NO. 123 | NS |
| 2C1 | Heavy | SEQ. | EVQLHQSGADLVK | SEQ. | GFHIKDT | SEQ | IHWMKQRPEQG | SEQ | IDPANG |
| | | NO. 52 | PGASVKLSCTAS | NO. 76 | Y | NO. 100 | LEWIGR | NO. 124 | NS |
| 1G1 | Heavy | SEQ. | QVQLQQPGAELVK | SEQ. | GYTFTSY | SEQ. | MHWVKQRPGQ | SEQ. | IDPSDSE |
| | | NO. 53 | PGASVKLSCKAS | NO. 77 | W | NO. 101 | GLEWIGN | NO. 125 | T |
| 1F2 | Heavy | SEQ. | QVOLKESGPVLVAP | SEQ. | GFSLTSY | SEQ. | IHWVRQPPGKG | SEQ. | IWAGGS |
| | | NO. 54 | SQSLSITCTVS | NO. 78 | A | NO. 102 | LEWLGV | NO. 126 | T |
| 2G7 | Heavy | SEQ. | QVQLQQSGAELAR | SEQ. | GYTFITY | SEQ. | MHWVKQRPGQ | SEQ. | INPSSDY |
| | | NO. 55 | PGASVKMSCKAS | NO. 79 | T | NO. 103 | GLEWIGY | NO. 127 | T |
| 1E10 | Heavy | SEQ. | QVQLKQSGPGLVQ | SEQ. | SFSLTSY | SEQ. | VHWVRQSPGK | SEQ. | IWSGVS |
| | | NO. 56 | PSQSLSITCTVS | NO. 80 | G | NO. 104 | GLEWLGV | NO. 128 | T |
| 1C4 | Heavy | SEQ. | QVQLKESGPGLVAP | SEQ. | GFSLTSY | SEQ. | VSWVRQPPGKG | SEQ. | IWGDGS |
| | | NO. 57 | SQSLSITCTVS | NO. 81 | G | NO. 105 | LEWLGV | NO. 129 | T |
| 2D4 | Heavy | SEQ. | QVQLQQSGAELAR | SEQ. | GYTFTSY | SEQ | ISWVKQRTGQG | SEQ. | IYPRSG |
| | | NO. 58 | PGASVKLSCKAS | NO. 82 | G | NO. 106 | LEWIGE | NO. 130 | NT |
| 1G10 | Heavy | SEQ. | EVKLVESGGGLVKP | SEQ. | GFTFSGY | SEQ. | MSWVRQTPAK | SEQ. | ISGSGG |
| | | NO. 59 | GGSLKLSCAAS | NO. 83 | T | NO. 107 | RLEWVAT | NO. 131 | NI |
| 1H2 | Heavy | SEQ. | EVKLVESGGGLVKP | SEQ. | GFTFSGY | SEQ. | MSWVRQTPAK | SEQ. | ISGSGG |
| | | NO. 60 | GGSLKLSCAAS | NO. 84 | T | NO. 108 | RLEWVAT | NO. 132 | NI |
| 1E12 | Heavy | SEQ. | QVQLQQPGSELVRP | SEQ. | GYIFTSY | SEQ. | MHWVKQRPGQ | SEQ. | IDPSDTY |
| | | NO. 61 | GTSVKLSCKAS | NO. 85 | W | NO. 109 | GLEWIGV | NO. 133 | T |
| 2B3 | Heavy | SEQ | EVKLVESGGGLVQP | SEQ. | GFTFSDY | SEQ | MYWVRQTPEK | SEQ. | ISNGGG |
| | | NO. 62 | GGSLKLSCAAS | NO. 86 | Y | NO. 110 | RLEWVAY | NO. 134 | ST |
| 2C9 | Heavy | SEQ. | EVQLQQSGPELVKP | SEQ. | GYTFKDY | SEQ. | MNWVKQSHGK | SEQ. | INPNNG |
| | | NO. 63 | GASVKISCKAS | NO. 87 | Y | NO. 111 | SLQWIGD | NO. 135 | GT |
| 1A1 | Heavy | SEQ. | EVQLQQSGPELVKP | SEQ. | AYTFTDY | SEQ. | MNWVKQSHGK | SEQ. | INPNNG |
| | | NO. 64 | GTSMKISCKAS | NO. 88 | F | NO. 112 | SLEWIGD | NO. 136 | AT |

TABLE 6

| Name | Chain | | FR3 | | CDR3 | | FR4 |
|---|---|---|---|---|---|---|---|
| 1F5 | Heavy | SEQ. | SYNQKFKGKATLTVDKSSST | SEQ. | ARGAY | SEQ. | WGQGTLVTVSA |
| | | NO. 137 | AYMELHSLTSEDSAVYYC | NO. 161 | | NO. 185 | |
| 2F1 | Heavy | SEQ. | RYNQKFKGKATLTVDKSSS | SEQ. | ARGGLFYAMDN | SEQ. | WGQGTSVTVSS |
| | | NO. 138 | TAYMELRSLTSEDSAVYFC | NO. 162 | | NO. 186 | |
| 1A7 | Heavy | SEQ. | IYNQKFKGKATLTVDKSSNT | SEQ. | ARRDYQSPFDY | SEQ. | WGQGTTLTVSS |
| | | NO. 139 | AYMELRSLTSEDTAVYYC | NO. 163 | | NO. 187 | |
| 1H5 | Heavy | SEQ | AYNQKFKDKAILIADKSSST | SEQ | TRAYYNHYVHFDY | SEQ. | WGQGTTLTVSS |
| | | NO. 140 | AYMELRSLTSEDSAVYYC | NO. 164 | | NO. 188 | |
| 1B12 | Heavy | SEQ. | EYNQKFKDKATLTADRSSST | SEQ. | VRGYFGNYDYALD | SEQ. | WGQGTSVTVSS |
| | | NO. 141 | AYMQLSSLTSEDSAVYYC | NO. 165 | Y | NO. 189 | |
| 1E2 | Heavy | SEQ. | HYNQKFKDKATLTVDKSSS | SEQ. | ARHYDGYAVDY | SEQ. | WGQGTSVTVSS |
| | | NO. 142 | TAYMQLSSLTSEDSAVYYC | NO. 166 | | NO. 190 | |
| 1A3 | Heavy | SEQ. | DYNSALMSRLNINKDNSKS | SEQ. | ARHSSKAYYSNYF | SEQ. | WGQGTTLTVSS |
| | | NO. 143 | QVFLKMNSLQIDDTAMYYC | NO. 167 | DY | NO. 191 | |
| 1D12 | Heavy | SEQ. | RYNPSLKSRLTISKDTSSNQV | SEQ. | ARTMITTRNYFDY | SEQ. | WGQGTTLTVSS |
| | | NO. 144 | FLKITSVDTADTATYYC | NO. 168 | | NO. 192 | |
| 1E7 | Heavy | SEQ. | YYPDSVKGRFTISRDNARNT | SEQ. | ARHGKIYDGYPFA | SEQ. | WGQGTSVTVSS |
| | | NO. 145 | LYLQMSSLRSEDTAMYYC | NO. 169 | MDF | NO. 193 | |
| 1H1 | Heavy | SEQ. | HYNQKFKDKATLTVDKSSS | SEQ. | ARHYDGYAVDY | SEQ. | WGQGTSVTVSS |
| | | NO. 146 | TAYMQLSSLTSEDSAVYYC | NO. 170 | | NO. 194 | |
| 2C2 | Heavy | SEQ. | EYDPKFQGKATITADTSSNT | SEQ. | AESNYFGSSPYALD | SEQ. | WGQGTSVTVSS |
| | | NO. 147 | AYLHLSSLTSEDTAVYYC | NO. 171 | Y | NO. 195 | |
| 2C1 | Heavy | SEQ. | EYDPKFQGKATITADTSSNT | SEQ. | AESNYFGSSPYALD | SEQ. | WGQGTSVTVSS |
| | | NO. 148 | AYLHLSSLTSEDTAVYYC | NO. 172 | Y | NO. 196 | |
| 1G1 | Heavy | SEQ. | HYNQKFKDKATLTVDKSSS | SEQ. | ARHYDGYAVDY | SEQ. | WGQGTSVTVSS |
| | | NO. 149 | TAYMQLSSLTSEDSAVYYC | NO. 173 | | NO. 197 | |
| 1F2 | Heavy | SEQ. | DYNSALMSRLNINKDNSKS | SEQ. | ARHSSKAYYSNYF | SEQ. | WGQGTTLTVSS |
| | | NO. 150 | QVFLKMNSLQIDDTAMYYC | NO. 174 | DY | NO. 198 | |
| 2G7 | Heavy | SEQ. | NYNQRFKDKATLTADKSSS | SEQ. | ARGAPAY | SEQ. | WGQGTLVTVSA |
| | | NO. 151 | TAYMQLNSLTSEDSAVYYC | NO. 175 | | NO. 199 | |
| 1E10 | Heavy | SEQ | DYNAAFISRLNISKDNSKSQ | SEQ. | ARNGLGLRRASMD | SEQ. | WGQGTSVTVSS |
| | | NO. 152 | VFFKMNSLQTDDTAIYYC | NO. 176 | Y | NO. 200 | |
| 1C4 | Heavy | SEQ. | NYHSALISRLSISKDNSKSQV | SEQ. | AKGGDGFDY | SEQ. | WGQGTTLTVSS |
| | | NO. 153 | FLKLNSLQTDDTATYYC | NO. 177 | | NO. 201 | |
| 2D4 | Heavy | SEQ. | YYNEKFKGKATLTADKSSST | SEQ. | ARSDY | SEQ. | WGQGTTLTVSS |
| | | NO. 154 | AYMELRSLTSEDSAVYFC | NO. 178 | | NO. 202 | |
| 1G10 | Heavy | SEQ. | YYPDTVKGRFTISRDNARNT | SEQ. | TRQEGGLRGFAY | SEQ. | WGQGTLVTVSA |
| | | NO. 155 | LYLQMSSLRSEDTAMYYC | NO. 179 | | NO. 203 | |
| 1H2 | Heavy | SEQ | YYPDTVKGRFTISRDNARNT | SEQ. | TRQEGGLRGFAY | SEQ. | WGQGTLVTVSA |
| | | NO. 156 | LYLQMSSLRSEDTAMYYC | NO. 180 | | NO. 204 | |
| 1E12 | Heavy | SEQ. | NYNQKFKGKATLTVDTSSST | SEQ. | AREEYGDYVGGFD | SEQ. | WGQGTTLTVSS |
| | | NO. 157 | AYMQLSSLTSEDSAVYYC | NO. 181 | F | NO. 205 | |
| 2B3 | Heavy | SEQ. | YYPDTVKGRFTISRDNAKNT | SEQ. | ARLAEDGYYGDYF | SEQ. | WGQGTTLTVSS |
| | | NO. 158 | LYLQMSRLKSEDTAMYYC | NO. 182 | DY | NO. 206 | |
| 2C9 | Heavy | SEQ. | TYNQKFKGKATLTVDKSSSI | SEQ. | ASDFDY | SEQ. | WGQGTTLTVSS |
| | | NO. 159 | AYMELRSLTSEDSAVYYC | NO. 183 | | NO. 207 | |
| 1A1 | Heavy | SEQ. | RYNQRFKVKATLTVDKSSST | SEQ. | ARGDEGFVY | SEQ. | WGQGTLVTVSA |
| | | NO. 160 | AYMELRSLTSEDSAVYYC | NO. 184 | | NO. 208 | |

TABLE 7

| | | Heavy V | Heavy D | Heavy J | Heavy C |
|---|---|---|---|---|---|
| Name | Chain | Gene | Gene | Gene | Gene |
| 1F5 | Heavy | IGHV1-34 | NA | IGHJ3 | IGHM |
| 2F1 | Heavy | IGHV1-26 | IGHD3-1; IGHD3-2 | IGHJ4 | IGHG2B |
| 1A7 | Heavy | IGHV1-18 | IGHD2-1; IGHD2-10; IGHD2-11; IGHD2-8 | IGHJ2 | IGHG2B |
| 1H5 | Heavy | IGHV1-15 | IGHD2-5; IGHD2-6 | IGHJ2 | IGHG2B |
| 1B12 | Heavy | IGHV1-7 | IGHD2-10; IGHD2-11 | IGHJ4 | IGHG2C |
| 1E2 | Heavy | IGHV1-52; IGHV1-61 | IGHD1-NCBI-FL16.1J | IGHJ4 | IGHG2C |
| 1A3 | Heavy | IGHV2-9 | IGHD2-5; IGHD2-6 | IGHJ2 | IGHG2C |
| 1D12 | Heavy | IGHV8-12 | IGHD2-4; IGHD2-9 | IGHJ2 | IGHG2C |
| 1E7 | Heavy | IGHV5-9 | IGHD2-3 | IGHJ4 | IGHG2C |
| 1H1 | Heavy | IGHV1-52; IGHV1-61 | IGHD1-NCBI-FL16.1J | IGHJ4 | IGHG2C |
| 2C2 | Heavy | IGHV14-3 | IGHD1-1 | IGHJ4 | IGHG2C |
| 2C1 | Heavy | IGHV14-3 | IGHD1-1 | IGHJ4 | IGHG2C |
| 1G1 | Heavy | IGHV1-52; IGHV1-61 | IGHD1-NCBI-FL16.1J | IGHJ4 | IGHG2C |
| 1F2 | Heavy | IGHV2-9 | IGHD2-5; IGHD2-6 | IGHJ2 | IGHG2C |
| 2G7 | Heavy | IGHV1-4 | IGHD3-1; IGHD3-2 | IGHJ3 | IGHG2C |
| 1E10 | Heavy | IGHV2-2 | IGHD3-1; IGHD3-2 | IGHJ4 | IGHG2C |
| 1C4 | Heavy | IGHV2-3 | IGHD4-1 | IGHJ2 | IGHG2C |
| 2D4 | Heavy | IGHV1-81 | IGHD1-NCBI-FL16.1J | IGHJ2 | IGHG2C |
| 1G10 | Heavy | IGHV5-9 | IGHD2-10; IGHD2-11; IGHD2-12; | IGHJ3 | IGHG1 |
| | | | IGHD2-14; IGHD2-5; IGHD2-6; IGHD2-9 | | |
| 1H2 | Heavy | IGHV5-9 | IGHD2-10; IGHD2-11; IGHD2-12; | IGHJ3 | IGHG1 |
| | | | IGHD2-14; IGHD2-5; IGHD2-6; IGHD2-9 | | |
| 1E12 | Heavy | IGHV1-59 | IGHD2-13 | IGHJ2 | IGHG2B |
| 2B3 | Heavy | IGHV5-12 | IGHD2-3 | IGHJ2 | IGHG1 |
| 2C9 | Heavy | IGHV1-26 | NA | IGHJ2 | IGHG1 |
| 1A1 | Heavy | IGHV1-26 | IGHD4-1 | IGHJ3 | IGHG1 |

TABLE 8

| Name | Chain | | FR1 | | CDR1 | | FR2 | | CDR2 |
|---|---|---|---|---|---|---|---|---|---|
| 1F5 | Light | SEQ | DIVMTQSQKFMSTS | SEQ. | QNVGTN | SEQ. | VAWYQQKP | SEQ. | SAS |
| | | NO. 209 | VGDRVSVTCKAS | NO. 235 | | NO. 261 | GOSPKALIY | NO. 287 | |
| 2F1 | Light | SEQ. | DIVMTQSPSSLSVSA | SEQ. | QSLLNSRNQKNY | SEQ. | LAWYQQKP | SEQ. | GAS |
| | | NO. 210 | GHKVTMSCKSS | NO. 236 | | NO. 262 | WQPPKLLIY | NO. 288 | |
| 1A7 | Light | SEQ. | DIQMTQTTSSLSASL | SEQ. | QGINNY | SEQ. | LNWYQQKP | SEQ. | YTS |
| | | NO. 211 | GDRVTISCSAS | NO. 237 | | NO. 263 | DGTVKLLIY | NO. 289 | |
| 1H5 | Light | SEQ | NIVLTQSPASLAVSL | SEQ. | ESVDSYGNSF | SEQ. | MHWYQQKP | SEQ. | LAS |
| | | NO. 212 | GQRATISCRAS | NO. 238 | | NO. 264 | GQPPKLLIY | NO. 290 | |
| 2H3 | Light | SEQ. | QIVLSQSPAILSASPG | SEQ. | SSVSY | SEQ. | MHWYQQKP | SEQ. | ATS |
| | | NO. 213 | EKVTMTCRAS | NO. 239 | | NO. 265 | GSSPKPWIY | NO. 291 | |
| 1B12 | Light | SEQ. | NIVLTQSPASLAVSL | SEQ. | ESVDSYGNSF | SEQ. | MHWYQQKP | SEQ. | LAS |
| | | NO. 214 | GQRATISCRAS | NO. 240 | | NO. 266 | GQPPKLLIY | NO. 292 | |
| 1E2 | Light | SEQ. | DIVLTQSPASLPVSL | SEQ | QSVTTSRYSY | SEQ. | MHWYQQKP | SEQ. | YAS |
| | | NO. 215 | GQRATISCRAS | NO. 241 | | NO. 267 | GQPPKLLIM | NO. 293 | |
| 1A3 | Light | SEQ. | DIQITQTTSSLSASLG | SEQ. | QGLSNY | SEQ. | LNWYQQKP | SEQ. | YTS |
| | | NO. 216 | DRVTISCSAS | NO. 242 | | NO. 268 | DGTVKLLIY | NO. 294 | |
| 1D12 | Light | SEQ. | DIVLTQSPASLAVSL | SEQ. | QSVDYDGDSY | SEQ. | MNWYQQKP | SEQ | AAS |
| | | NO. 217 | GQRATISCKAS | NO. 243 | | NO. 269 | GQPPKLLIY | NO. 295 | |
| 1E7 | Light | SEQ. | DIQMTQSPASLSVSV | SEQ. | ENIYSN | SEQ. | LAWYHQKQ | SEQ. | AAT |
| | | NO. 218 | GETVTITCRAS | NO. 244 | | NO. 270 | GKSPQLLVY | NO. 296 | |
| 1H1 | Light | SEQ. | DIVLTQSPASLPVSL | SEQ. | QSVTTSRYSY | SEQ. | MHWYQQKP | SEQ. | YAS |
| | | NO. 219 | GQRATISCRAS | NO. 245 | | NO. 271 | GQPPKLLIM | NO. 297 | |
| 2C2 | Light | SEQ. | DIILTQSPATLSVTPG | SEQ. | QSISNN | SEQ | LHWYQQKS | SEQ. | YAS |
| | | NO. 220 | DSVSLSCRAS | NO. 246 | | NO. 272 | HESPRLLIK | NO. 298 | |
| 2C1 | Light | SEQ. | DIVMTQSHKFMSTS | SEQ | QDVGTA | SEQ. | VAWYQHKP | SEQ | WAS |
| | | NO. 221 | VGDRVSISCKAS | NO. 247 | | NO. 273 | GQSPKVLIY | NO. 299 | |
| 1G1 | Light | SEQ. | DIVLTQSPASLPVSL | SEQ. | QSVTTSRYSY | SEQ. | MHWYQQKP | SEQ. | YAS |
| | | NO. 222 | GQRATISCRAS | NO. 248 | | NO. 274 | GQPPKLLIM | NO. 300 | |
| 1F2 | Light | SEQ. | DIQITQTTSSLSASLG | SEQ. | QGLSNY | SEQ. | LNWYQQKP | SEQ. | YTS |
| | | NO. 223 | DRVTISCSAS | NO. 249 | | NO. 275 | DGTVKLLIY | NO. 301 | |
| 2G7c | Light | SEQ. | DIILTQSPATLSVTPG | SEQ | QSISNN | SEQ. | LHWYQQKS | SEQ. | YAS |
| | | NO. 224 | DSVSLSCRAS | NO. 250 | | NO. 276 | HESPRLLIK | NO. 302 | |
| 1E10 | Light | SEQ. | QIVLSQSPAILSASPG | SEQ. | SSVSY | SEQ | MHWYQQKP | SEQ. | ATS |
| | | NO. 225 | EKVTMTCRAS | NO. 251 | | NO. 277 | GSSPKPWIY | NO. 303 | |
| 1C4 | Light | SEQ. | DIVLTQSPASLAVSL | SEQ. | ESVSIRGTSL | SEQ. | MHWYQQKP | SEQ. | AAS |
| | | NO. 226 | GQRATISCRAS | NO. 252 | | NO. 278 | GYPPKLLIY | NO. 304 | |
| 2D4 | Light | SEQ. | DIVMTQSQKFMSTS | SEQ. | QNVGTN | SEQ. | VAWYQQKP | SEQ. | SAS |
| | | NO. 227 | VGDRVSVTCKAS | NO. 253 | | NO. 279 | GQSPKALIY | NO. 305 | |
| 1B8 | Light | SEQ. | DIVLTQSPASLAVSL | SEQ. | ESVDNYGISF | SEQ. | MNWFQQKP | SEQ. | AAS |
| | | NO. 228 | GQRATISCRAS | NO. 254 | | NO. 280 | GQPPKLLIY | NO. 306 | |
| 1G10 | Light | SEQ. | DIQMTQSPASLSVSV | SEQ | ENIYNN | SEQ. | LAWYQQKQ | SEQ. | AAT |
| | | NO. 229 | GETVTITCRAS | NO. 255 | | NO. 281 | GKSPQLLVY | NO. 307 | |
| 1H2 | Light | SEQ. | DIQMTQSPASLSVSV | SEQ. | ENIYNN | SEQ. | LAWYQQKQ | SEQ. | AAT |
| | | NO. 230 | GETVTITCRAS | NO. 256 | | NO. 282 | GKSPQLLVY | NO. 308 | |
| 1E12 | Light | SEQ. | DIVLTQSPASLAVSL | SEQ. | QSVSTSRYSY | SEQ. | MYWYQQKP | SEQ. | YAS |
| | | NO. 231 | WQRATISCRAS | NO. 257 | | NO. 283 | GQPPKLLIK | NO. 309 | |
| 2B3 | Light | SEQ. | DIQMTQTTSSLSASL | SEQ | QDISNY | SEQ. | LNWYQQKP | SEQ. | YTS |
| | | NO. 232 | GDRVTISCRAS | NO. 258 | | NO. 284 | DGTVKLLIY | NO.310 | |
| 2C9 | Light | SEQ. | DVVMTQTPLSLPVSL | SEQ. | QSLVYSNGNTY | SEQ. | LHWYLQKPG | SEQ. | KVS |
| | | NO. 233 | GDQASISCRSS | NO. 259 | | NO. 285 | QSPKLLIY | NO. 311 | |
| 1A1 | Light | SEQ. | DIVMTQSPSSLSVSA | SEQ. | QSLLNSRNQKNY | SEQ. | LAWYQQKP | SEQ. | GAS |
| | | NO. 234 | GDKVTMSCKSS | NO. 260 | | NO. 286 | WQPPKLLIY | NO. 312 | |

TABLE 9

| Name | Chain | | FR3 | | CDR3 | | FR4 |
|---|---|---|---|---|---|---|---|
| 1F5 | Light | SEQ. | YRYSGVPDRFTGSGSGTDFTLTI | SEQ. | QQYNNYPYT | SEQ | FGGGTKLEIK |
| | | NO. 313 | SNVQSEDLAEYFC | NO. 339 | | NO. 365 | |
| 2F1 | Light | SEQ. | TRESGVPDRFTGSGSGTDFTLTI | SEQ. | QNDYSYPPT | SEQ | FGAGTKLELK |
| | | NO. 314 | SSVQAEDLAVYYC | NO. 340 | | NO. 366 | |
| 1A7 | Light | SEQ. | SLHSGVPSRFSGSGSGTDYSLTIS | SEQ. | QQYSKLPFT | SEQ. | FGSGTKLEIK |
| | | NO. 315 | NLEPEDIATYYC | NO. 341 | | NO. 367 | |
| 1H5 | Light | SEQ. | NLESGVPARFSGSGSRTDFTLTI | SEQ. | QQNNEDPWT | SEQ | FGGGTKLEIK |
| | | NO. 316 | DPVEADDAATYYC | NO. 342 | | NO. 368 | |
| 2H3 | Light | SEQ. | NLASGVPARFSGSGSGTSYSLTI | SEQ. | QQWSSNPPT | SEQ | FGGGTKLEIK |
| | | NO. 317 | SRVEAEDAATYYC | NO. 343 | | NO. 369 | |
| 1B12 | Light | SEQ. | NLESGVPARFSGSGSRTDFTLTI | SEQ. | QQNNEDPWT | SEQ. | FGGGTKLEIK |
| | | NO. 318 | DPVEADDAATYYC | NO. 344 | | NO. 370 | |
| 1E2 | Light | SEQ. | NLESGVPARFSGSGSGTDFTLNI | SEQ. | QHSWEIPWT | SEQ. | FGGGTKLEIK |
| | | NO. 319 | HPVEEEDIAIYYC | NO. 345 | | NO. 371 | |
| 1A3 | Light | SEQ. | ILHSGVPSRFSGSGSGTDYSLTIS | SEQ. | XQYSKLPWT | SEQ. | FGGGSKLEIK |
| | | NO. 320 | NLEPEDIATYYC | NO. 346 | | NO. 372 | |
| 1D12 | Light | SEQ. | NLESGIPARFSGSGSGTDFTLNIH | SEQ. | QQSNEDPP | SEQ | FGGGTKLEIK |
| | | NO. 321 | PVEEEDAATYYC | NO. 347 | | NO. 373 | |
| 1E7 | Light | SEQ. | NLADGVPSRFSGNGSGTQYSLKI | SEQ. | QHFWGTPWT | SEQ | FGGGTKLEIK |
| | | NO. 322 | NSLQSEDFGSYYC | NO. 348 | | NO. 374 | |
| 1H1 | Light | SEQ. | NLESGVPARFSGSGSGTDFTLNI | SEQ. | QHSWEIPWT | SEQ. | FGGGTKLEIK |
| | | NO. 323 | HPVEEEDIAIYYC | NO. 349 | | NO. 375 | |
| 2C2 | Light | SEQ. | QSISGIPSRFSGSGSGTDFTLSINS | SEQ. | QQSNSWPLT | SEQ. | FGAGTKLELK |
| | | NO. 324 | VETEDFGMYFC | NO. 350 | | NO. 376 | |
| 2C1 | Light | SEQ | NRHTGVPDRFTGSGSGTDFTLTI | SEQ. | QQYSYYPT | SEQ | FGGGTKLEIK |
| | | NO. 325 | TNVQSEDLADYFC | NO. 351 | | NO. 377 | |
| 1G1 | Light | SEQ | NLESGVPARFSGSGSGTDFTLNI | SEQ. | QHSWEIPWT | SEQ. | FGGGTKLEIK |
| | | NO. 326 | HPVEEEDIAIYYC | NO. 352 | | NO. 378 | |
| 1F2 | Light | SEQ. | ILHSGVPSRFSGSGSGTDYSLTIS | SEQ. | QQYSKLPWT | SEQ. | FGGGSKLEIK |
| | | NO. 327 | NLEPEDIATYYC | NO. 353 | | NO. 379 | |
| 2G7 | Light | SEQ. | QSISGIPSRFSGSGSGTDFTLSINS | SEQ. | QQSNSWPLT | SEQ. | FGAGTKLELK |
| | | NO. 328 | VETEDFGMYFC | NO. 354 | | NO. 380 | |
| 1E10 | Light | SEQ. | NLASGVPARFSGSGSGTSYSLTI | SEQ. | QQWSSNPPT | SEQ | FGGGTKLEIK |
| | | NO. 329 | SRVEAEDAATYYC | NO. 355 | | NO. 381 | |
| 1C4 | Light | SEQ. | NLESGVPARFSGRRSGTDFTLNI | SEQ. | QQSREYPLT | SEQ | FGAGTKLELK |
| | | NO. 330 | HPVEEDDAATYYC | NO. 356 | | NO. 382 | |
| 2D4 | Light | SEQ. | YRYSGVPDRFTGSGSGTDFTLTI | SEQ. | QQYNSYPYT | SEQ | FGGGTKLEIK |
| | | NO. 331 | SNVQSEDLAEYFC | NO. 357 | | NO. 383 | |
| 1B8 | Light | SEQ. | NQGSGVPARFSGSGSGTDFSLNI | SEQ. | QQSKEVPFT | SEQ. | FGSGTKLEIK |
| | | NO.332 | HPMEEDDTAMYFC | NO. 358 | | NO. 384 | |
| 1G10 | Light | SEQ. | NLADGVPSRFSGSGSGTQFSLKI | SEQ. | QHFWGIPYT | SEQ | FGGGTKLEIT |
| | | NO. 333 | NSLQSEDFGSYYC | NO. 359 | | NO. 385 | |
| 1H2 | Light | SEQ. | NLADGVPSRFSGSGSGTQFSLKI | SEQ. | QHFWGIPYT | SEQ | FGGGTKLEIT |
| | | NO. 334 | NSLQSEDFGSYYC | NO. 360 | | NO. 386 | |
| 1E12 | Light | SEQ. | NLESGVPARFSGSGSGTDFTLNI | SEQ. | QHSWEIPYT | SEQ. | FGGGTMLEIK |
| | | NO. 335 | HPVEEEDTATYYC | NO. 361 | | NO. 387 | |
| 2B3 | Light | SEQ. | RLHSGVPSRFSGSGSGTDYSLTI | SEQ. | QQYSKLPLT | SEQ. | FGAGTKLELK |
| | | NO. 336 | SNLEPEDIATYYC | NO. 362 | | NO. 388 | |
| 2C9 | Light | SEQ. | NRFSGVPDRFSGSGSGTDFTLKI | SEQ. | SQSTHVPFT | SEQ. | FGSGTKLEIK |
| | | NO. 337 | SRVEAEDLGVYFC | NO. 363 | | NO. 389 | |
| 1A1 | Light | SEQ. | TRESGVPDRFTGSGSGTDFTLTI | SEQ | QNDYSYPPT | SEQ | FGGGTKLEIK |
| | | NO. 338 | SSVQAEDLAVYYC | NO. 364 | | NO. 390 | |

TABLE 10

| Name | Chain | Light V Gene | Light J Gene | Light C Gene |
|---|---|---|---|---|
| 1F5 | Light | IGKV6-15 | IGKJ2 | IGKC |
| 2F1 | Light | IGKV8-28 | IGKJ5 | IGKC |
| 1A7 | Light | IGKV10-94 | IGKJ4 | IGKC |
| 1H5 | Light | IGKV3-10 | IGKJ1 | IGKC |
| 2H3 | Light | IGKV4-72 | IGKJ1 | IGKC |
| 1B12 | Light | IGKV3-10 | IGKJ1 | IGKC |
| 100 | Light | IGKV3-7 | IGKJ1 | IGKC |
| 1A3 | Light | IGKV10-94 | IGKJ1 | IGKC |
| 1D12 | Light | IGKV3-4 | IGKJ2 | IGKC |
| 1E7 | Light | IGKV12-46 | IGKJ1 | IGKC |
| 1H1 | Light | IGKV3-7 | IGKJ1 | IGKC |
| 2C2 | Light | IGKV5-43 | IGKJ5 | IGKC |
| 2C1 | Light | IGKV6-23 | IGKJ2 | IGKC |
| 1G1 | Light | IGKV3-7 | IGKJ1 | IGKC |
| 1F2 | Light | IGKV10-94 | IGKJ1 | IGKC |
| 2G7 | Light | IGKV5-43 | IGKJ5 | IGKC |
| 1E10 | Light | IGKV4-72 | IGKJ1 | IGKC |
| 1C4 | Light | IGKV3-9 | IGKJ5 | IGKC |
| 2D4 | Light | IGKV6-15 | IGKJ2 | IGKC |
| 1B8 | Light | IGKV3-2 | IGKJ4 | IGKC |
| 1G10 | Light | IGKV12-46 | IGKJ2 | IGKC |
| 1H2 | Light | IGKV12-46 | IGKJ2 | IGKC |
| 1E12 | Light | IGKV3-7 | IGKJ2 | IGKC |
| 2B3 | Light | IGKV10-94 | IGKJ5 | IGKC |
| 2C9 | Light | IGKV1-110 | IGKJ4 | IGKC |
| 1A1 | Light | IGKV8-28 | IGKJ1 | IGKC |

What is claimed is:

1. An antigen-binding molecule that specifically binds to SEMA-4A, wherein the antigen-binding molecule comprises an immunoglobulin heavy chain domain (HC) and an immunoglobulin light chain domain (LC), wherein the HC or LC comprises a complementarity determining region, and wherein the HC comprises an amino acid sequence having at least 80% sequence identity to one of SEQ ID NO: 11-19, and the LC comprises an amino acid sequence having at least 80% sequence identity to one of SEQ ID NO: 1-10.

2. An antigen-binding molecule of claim 1, wherein the HC comprises an amino acid sequence having at least 85% sequence identity to one of SEQ ID NO: 11-19, and the LC comprises an amino acid sequence having at least 85% sequence identity to one of SEQ ID NO: 1-10.

3. An antigen-binding molecule of claim 1, wherein the HC comprises an amino acid sequence having at least 90% sequence identity to one of SEQ ID NO: 11-19, and the LC comprises an amino acid sequence having at least 90% sequence identity to one of SEQ ID NO: 1-10.

4. An antigen-binding molecule of claim 1, wherein the HC comprises an amino acid sequence having at least 95% sequence identity to one of SEQ ID NO: 11-19, and the LC comprises an amino acid sequence having at least 95% sequence identity to one of SEQ ID NO: 1-10.

5. An antigen-binding molecule of claim 1, wherein the HC comprises an amino acid sequence having at least 95% sequence identity to one of SEQ ID NO: 11-19.

6. An antigen-binding molecule of claim 1, wherein the LC comprises an amino acid sequence having at least 95% sequence identity to one of SEQ ID NO: 1-10.

7. The antigen-binding molecule of claim 1, wherein the antigen-binding fragment is comprised in a target binding modality including but not limited to an ADC, a bi-specific engager, and a tri-specific engager.

8. The antigen-binding molecule of claim 1, wherein the antigen-binding fragment is engineered into one or more cell types including but not limited to T cells, NK cells, macrophages, and pluripotent stem cells.

9. A pharmaceutical composition comprising the antigen-binding molecule of claim 1.

10. A method for treating multiple myeloma, said method comprising administering the pharmaceutical composition of claim 1 to a patient in need of such treatment.

11. An antigen-binding molecule that specifically binds to FCRL3, wherein the antigen-binding molecule comprises an immunoglobulin heavy chain domain (HC) and an immunoglobulin light chain domain (LC), wherein the HC or LC comprises a complementarity determining region, and wherein the HC comprises an amino acid sequence having at least 80% sequence identity to one of SEQ ID NO: 31-40, and the LC comprises an amino acid sequence having at least 80% sequence identity to one of SEQ ID NO: 20-30.

12. An antigen-binding molecule of claim 11, wherein the HC comprises an amino acid sequence having at least 85% sequence identity to one of SEQ ID NO: 31-40, and the LC comprises an amino acid sequence having at least 85% sequence identity to one of SEQ ID NO: 20-30.

13. An antigen-binding molecule of claim 11, wherein the HC comprises an amino acid sequence having at least 90% sequence identity to one of SEQ ID NO: 31-40, and the LC comprises an amino acid sequence having at least 90% sequence identity to one of SEQ ID NO: 20-30.

14. An antigen-binding molecule of claim 11, wherein the HC comprises an amino acid sequence having at least 95% sequence identity to one of SEQ ID NO: 31-40, and the LC comprises an amino acid sequence having at least 95% sequence identity to one of SEQ ID NO: 20-30.

15. An antigen-binding molecule of claim 11, wherein the HC comprises an amino acid sequence having at least 95% sequence identity to one of SEQ ID NO: 20-30.

16. An antigen-binding molecule of claim 11, wherein the LC comprises an amino acid sequence having at least 95% sequence identity to one of SEQ ID NO: 31-40.

17. The antigen-binding molecule of claim 11, wherein the antigen-binding fragment is comprised in a target binding modality including but not limited to an ADC, a bi-specific engager, and a tri-specific engager.

18. The antigen-binding molecule of claim 11, wherein the antigen-binding fragment is engineered into one or more cell types including but not limited to T cells, NK cells, macrophages, and pluripotent stem cells.

19. A pharmaceutical composition comprising the antigen-binding molecule of claim 11.

20. A method for treating multiple myeloma, said method comprising administering the pharmaceutical composition of claim 11 to a patient in need of such treatment.


SEQUENCE LISTING (379 sequences)

SEQ ID NO: 1 - DNA, 482 bp, synthetic construct
  source 1..482: mol_type = other DNA
atggagaaagacacactcctgctatgggtcctgcttctctgggttccaggttccacaggtgacattgtgctgacccaatctccagcttctttggctgtgtctctagggcagagggccaccatctcctgcagagccagcgaaagtgttgataattatggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccattcacgttcggctcggggacaaagttggaaataaaacgggctgatgctgcaccaactgtatccatcttcccaccatccagtgagcagttaacatctggaggtgcctcagtcgtgtgcttcttgaa

SEQ ID NO: 2 - DNA, 399 bp, synthetic construct
  source 1..399: mol_type = other DNA
atggagacacattctcaggtctttgtatacatgttgctgtggttgtctgatgttgaaggtgacattgtgatgacccagtctcacaaattcatgtccacatcagtaggagacagggtcagcatctcctgcaaggccagtcaggatgtgggtactgctgttgcctggtatcaacataaaccaggacagtctcctaaagttctgatttactgggcatccaaccggcacactggagtccctgatcgcttcacaggcagtggatctgggacagatttcactctcaccattaccaatgtgcagtctgaagacttggcagattatttctgtcagcaatatagctactatcccacgttcggaggggggaccaagctggaaataaagcgggctgatgctgcaccaact

SEQ ID NO: 3 - DNA, 648 bp, synthetic construct
  source 1..648: mol_type = other DNA
atgtcctctgctcagttccttggtctcctgttgctctgttttcaaggtgtcagatgtgatatccagataacacagactacatcctccctgtctgcctctctgggagacagagtcaccatcagttgcagtgcaagtcagggccttagcaattatttaaactggtatcagcagaagccagatggaactgttaaactcctgatctattacacatcaattttacactcaggagtcccatcaaggttcagtggcagtgggtctgggacagattattctctcaccatcagcaacctggaacctgaagatattgccacttactattgtmagcagtatagtaagcttccgtggacgttcggtggaggctccaagttggaaatcaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccact

SEQ ID NO: 4 - DNA, 554 bp, synthetic construct
  source 1..554: mol_type = other DNA
atgagtgtgcccactcaggtcctggggttgctgctgctgtggcttacagatgccagatgtgacatccagatgactcagtctccagcctccctatctgtatctgtgggagaaactgtcaccatcacatgtcgagcaagtgagaatatttacagtaatttagcatggtatcaccagaaacagggaaaatctcctcaactcctggtctatgctgcaacaaacttagcagatggtgtgccatcaaggttcagtggcaatggatcaggcacacagtattccctcaagatcaacagcctgcagtctgaagattttgggagttattactgtcaacatttttggggtactccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactga

SEQ ID NO: 5 - DNA, 703 bp, synthetic construct
  source 1..703: mol_type = other DNA
atggagacagacacactcctgctatgggtgctgctgctctgggttccaggttccactggtgacattgtgctgacacagtctcctgcttccttacctgtatctctggggcagagggccaccatttcatgtagggccagccaaagtgtcactacatctaggtatagttatatgcactggtaccaacagaaaccaggacagccacccaaactcctcatcatgtatgcatccaacctagaatctggggtccctgccaggttcagtggcagtgggtctgggacagacttcaccctcaacatccatcctgtggaggaggaggatattgcaatatattactgtcagcacagttgggagatwccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaaca

SEQ ID NO: 6 - DNA, 524 bp, synthetic construct
  source 1..524: mol_type = other DNA
atggagacagacacactcctgctatgggtgctgctgcyctgggttccaggttccactggtgacattgtgctgacacagtctcctgcttccttacctgtatctctggggcagagggccaccatttcatgtagggccagccaaagtgtcactacatctaggtatagttatatgcactggtaccaacagaaaccaggacagccacccaaactcctcatcatgtatgcatccaacctagaatctggggtccctgccaggttcagtggcagtgggtctgggacagacttcaccctcaacatccatcctgtggaggaggaggatattgcaatatattactgtcagcacagttgggagattccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccagagacatcaatgtcaagtggaagattga

SEQ ID NO: 7 - DNA, 591 bp, synthetic construct
  source 1..591: mol_type = other DNA
atggagacagacacactcctgctatgggtgctgctgctctgggttccaggttccactggtgacattgtgctgacacagtctcctgcttccttacctgtatctctggggcagagggccaccatttcatgtagggccagccaaagtgtcactacatctaggtatagttatatgcactggtaccaacagaaaccaggacagccacccaaactcctcatcatgtatgcatccaacctagaatctggggtccctgccaggttcagtggcagtgggtctgggacagacttcaccctcaacatccatcctgtggaggaggaggatattgcaatatattactgtcagcacagttgggagattccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctac

SEQ ID NO: 8 - DNA, 515 bp, synthetic construct
  source 1..515: mol_type = other DNA
atgagtgtgcccactcaggtcctggggttgctgctgctgtggcttacagatgccagatgtgacatccagatgactcagtctccagcctccctatctgtatctgtgggagaaactgtcaccatcacatgtcgagcaagtgagaatatttacaataatttagcatggtatcagcagaaacagggaaaatctcctcagctcctggtctatgctgcaacaaacttagcagatggtgtgccatcaaggttcagtggcagtggatcaggcacacagttttccctcaagatcaacagcctgcagtctgaagattttgggagttattactgtcaacatttttggggtattccgtacacgttcggaggggggaccaagctggaaataacacgggctgatgctgcaccaactgtatccatcttcccaccatccagtgagcagttaacatctggaggtgcctcagtcgtgtgcttcttgaacaacttctaccccagagacatcaatgtcaagtggaagattgatgg

SEQ ID NO: 9 - DNA, 556 bp, synthetic construct
  source 1..556: mol_type = other DNA
atggttttcacacctcagatacttggacttatgcttttttggatttcagcctccagaggtgatattatactaactcagtctccagccaccctgtctgtgactccaggagatagcgtcagtctttcctgcagggccagccagagtattagcaacaacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcaacagagtaacagctggccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatc

SEQ ID NO: 10 - DNA, 638 bp, synthetic construct
  source 1..638: mol_type = other DNA
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatcctccgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataa

SEQ ID NO: 11 - DNA, 534 bp, synthetic construct
  source 1..534: mol_type = other DNA
atgaattgcagctggattatcttcttcctgatggcagtggttacaggggtcaattcagaggttcagctgcaccagtctggggcagaccttgtgaagccaggggcctcagtcaagttgtcctgcacagcttctggcttccacattaaagacacctatatacactggatgaaacagaggcctgaacagggcctggagtggattggaaggattgatcctgcgaatggtaattctgaatatgacccgaagttccagggcaaggccactataacagcagacacatcctccaacacagcctacctgcacctcagcagcctgacatctgaggacactgccgtctattactgtgctgaatccaattacttcggtagtagtccctatgctttggactactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtg

SEQ ID NO: 12 - DNA, 480 bp, synthetic construct
  source 1..480: mol_type = other DNA
atggctgttctggtgctgttcctctgcctggttgcatttccaagctgtgtcctgtcccaggtgcagctgaaggagtcaggacctgtcctggtggcgccctcacagagcctgtccatcacttgcactgtctctgggttttcattaaccagctatgctatacactgggttcgccagcctccaggaaagggtctggagtggctgggagtaatctgggctggtggaagcacagattataattcagctctcatgtccagactaaacatcaacaaagacaactccaagagccaagttttcttaaaaatgaacagtctgcaaattgatgacacagccatgtactactgtgccagacactcatcaaaagcctactatagtaactactttgactactggggccaaggcaccactctcacagtctcctcagccaaaacaacagccccatcggtctatccactggcccctgtgtgtggagatacaagtggc

SEQ ID NO: 13 - DNA, 489 bp, synthetic construct
  source 1..489: mol_type = other DNA
atgaacttcgggctcagcttgattttccttgtccttgttttaaaaggtgtccagtgtgaagtgaagctggtggagtctgggggaggcttagtgaagcctggagggtccctgaatctctcctgtgcagcctctggattcactttcaatagctttaccatgtcttgggttcgccagactccggcgaagaggctggagtgggtcgcaaccattagtagtagtggttataacacctactatccagacagtgtgaagggccgattcaccatctccagagacaatgccaggaacaccctgtacctgcaaatgagcagtctgaggtctgaggacacggccatgtattactgtgcaagacatgggaagatctatgatggttacccatttgctatggacttctggggtcaaggaacctcagtcaccgtctcctcagccaaaacaacagccccatcggtctatccactggcccctgtgtgtggagatacaagtggctcc

SEQ ID NO: 14 - DNA, 592 bp, synthetic construct
  source 1..592: mol_type = other DNA
atgggatggagctgtatcatcctctttttggtagcagcagctacaggtgtccactcccaggtccaactgcagcagcctggggctgagctggtgaagcctggggcttcagtgaagctgtcctgcaaggcttctggctacaccttcaccagctactggatgcactgggtgaagcagaggcctggacagggccttgaatggattggtaatattgacccttctgatagtgaaactcattacaatcaaaagttcaaggacaaggccacattgactgtagacaaatcttccagcacagcctacatgcaactcagcagcctgacatctgaggactctgcggtctattactgtgcaagacactacgatggctatgctgtggactactggggtcaaggaacctcagtcaccgtctcctcagccaaaacaacagccccatcggtctatccactggcccctgtgtgtggagatacaagtggctcctcggtgactctaggatgcctggtcaagggttatttccctgagccagtgaccttgacctggaactctggatccctgtccagtggtgtgcacaccttcccagctgtcctgcagtctgacc

SEQ ID NO: 15 - DNA, 544 bp, synthetic construct
  source 1..544: mol_type = other DNA
atgggatggagctgtatcatcctctttttggtagcagcagctacaggtgtccactcccaggtccaactgcagcagcctggggctgagctggtgaagcctggggcttcagtgaagctgtcctgcaaggcttctggctacaccttcaccagctactggatgcactgggtgaagcagaggcctggacagggccttgaatggattggtaatattgacccttctgatagtgaaactcattacaatcaaaagttcaaggacaaggccacattgactgtagacaaatcttccagcacagcctacatgcaactcagcagcctgacatctgaggactctgcggtctattactgtgcaagacactacgatggctatgctgtggactactggggtcaaggaacctcagtcaccgtctcctcagccaaaacaacagccccatcggtctatccactggcccctgtgtgtggagatacaagtggctcctcggtgactctaggatgcctggtcaagggttatttccctgagccagtgaccttgacctggaactctggat

SEQ ID NO: 16 - DNA, 582 bp, synthetic construct
  source 1..582: mol_type = other DNA
atgggatggagctgtatcatcctctttttggtagcagcagctacaggtgtccactcccaggtccaactgcagcagcctggggctgagctggtgaagcctggggcttcagtgaagctgtcctgcaaggcttctggctacaccttcaccagctactggatgcactgggtgaagcagaggcctggacagggccttgaatggattggtaatattgacccttctgatagtgaaactcattacaatcaaaagttcaaggacaaggccacattgactgtagacaaatcttccagcacagcctacatgcaactcagcagcctgacatctgaggactctgcggtctattactgtgcaagacactacgatggctatgctgtggactactggggtcaaggaacctcagtcaccgtctcctcagccaaaacaacagccccatcggtctatccactggcccctgtgtgtggagatacaagtggctcctcggtgactctaggatgcctggtcaagggttatttccctgagccagtgaccttgacctggaactctggatccctgtccagtggtgtgcacaccttcccagctgtcctg

SEQ ID NO: 17 - DNA, 449 bp, synthetic construct
  source 1..449: mol_type = other DNA
atgaacttcgggctcagcttgattttccttgtccttgttttaaaaggtgtccagtgtgaagtgaaactggtggagtctgggggaggcttagtgaagcctggagggtccctgaaactctcctgtgcagcctctggattcactttcagtggctataccatgtcttgggttcgccagactccggcgaagaggctggagtgggtcgcaaccattagtggttctggtggtaatatttactatccagacactgtgaagggccgattcaccatctccagagacaatgccaggaacaccctgtacctgcaaatgagcagtctgaggtctgaggacacggccatgtattactgtacaagacaggagggaggcttacgcggctttgcttactggggccaagggactctggtcactgtctctgcagccaaaacgacacccccatctgtctatccactggc

SEQ ID NO: 18 - DNA, 563 bp, synthetic construct
  source 1..563: mol_type = other DNA
atggaaaggcactggatctttctactcctgttgtcagtaactgcaggtgtccactcccaggtccagctgcagcagtctggggctgaactggcaagacctggggcctcagtgaagatgtcctgcaaggcttctggctacacctttattacctacacgatgcactgggtaaaacagaggcctggacagggtctggaatggattggatacattaatcctagcagtgattatactaattacaatcagagattcaaggacaaggccacattgactgcagacaaatcctccagcacagcctacatgcaactgaacagcctgacatctgaggactctgcagtctattactgtgcaaggggggctccggcttactggggccaagggactctggtcactgtctctgcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagc

SEQ ID NO: 19 - DNA, 514 bp, synthetic construct
  source 1..514: mol_type = other DNA
atgaacaggcttacttcctcattgctgctgctgattgtccctgcatatgtcctgtcccaggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagcaaccaggtattcctcaagatcaccagtgtggacactgcagatactgccacatactactgtgctcgtactatgattacgacgaggaactactttgactactggggccaaggcaccactctcacagtctcctcagccaaaacaacagccccatcggtctatccactggcccctgtgtgtggagatacaactggctcctcggtgactctaggatgcctggtcaagggtt

SEQ ID NO: 20 - DNA, 459 bp, synthetic construct
  source 1..459: mol_type = other DNA
atggaatcacagactcaggtcctcatgtccctgctgctctgggtatctggtacctgtggggacattgttatgacacagtctccatcctccctgagtgtgtcagcaggacataaggtcactatgagctgcaagtccagtcagagtctattaaacagtagaaatcaaaagaactacttggcctggtaccagcagaaaccatggcagcctcctaaacttctgatctacggggcatccactagggaatctggggtccctgatcgcttcacaggcagtggatctggaacagatttcactctcaccatcagcagtgtgcaggctgaagacctggcagtttattactgtcagaatgattatagttatcctcccacgttcggtgctgggaccaagctggagctgaaacgggctgatgctgcaccaactgtatccatcttcccaccatccagtgagcagttaacatct

SEQ ID NO: 21 - DNA, 506 bp, synthetic construct
  source 1..506: mol_type = other DNA
atggattttcaagtgcagattttcagcttcctgctaatcagtgcttcagtcataatgtccagaggacaaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagtagtaacccaccgacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtg

SEQ ID NO: 22 - DNA, 518 bp, synthetic construct
  source 1..518: mol_type = other DNA
atgaagttgcctgttaggctgttggtgctgatgttctggattcctggttccagcagtgatgttgtgatgacccaaactccactctccctgcctgtcagtcttggagatcaagcctccatctcttgcagatctagtcagagccttgtatacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaa

SEQ ID NO: 23 - DNA, 454 bp, synthetic construct
  source 1..454: mol_type = other DNA
atggagacagtcacactcctgctatgggtgctactgctctgggttccaggctccactggtgacattgtgctgacccaatctccagcttctttggctgtgtctctagggcagagggccaccatctcctgcagagccagcgaaagtgtcagtattcgtggtactagtttaatgcactggtaccaacagaaaccaggatatccacccaaactcctcatctatgctgcatccaacctagaatctggggttcctgccaggtttagtggcagaaggtctgggacagacttcaccctcaacattcatcctgtggaggaagatgatgctgcaacctattactgtcagcaaagtagggaatatccgctcacgttcggtgctgggaccaagctggagctgaaacgggctgatgctgcaccaactgtatccatcttcccaccatccagtgagcagttaacatctg

SEQ ID NO: 24 - DNA, 555 bp, synthetic construct
  source 1..555: mol_type = other DNA
atgatgtcctctgctcagttccttggtctcctgttgctctgttttcaaggtaccagatgtgatatccagatgacacagactacatcctccctgtctgcctctctgggagacagagtcaccatcagttgcagtgcaagtcagggcattaacaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgat

SEQ ID NO: 25 - DNA, 598 bp, synthetic construct
  source 1..598: mol_type = other DNA
atggaatcacagactcaggtcctcatgtccctgctgctctgggtatctggtacctgtggggacattgtgatgacacagtctccatcctccctgagtgtgtcagcaggagataaggtcactatgagctgcaagtccagtcagagtctgttaaacagtagaaaccaaaagaactacttggcctggtaccagcagaaaccatggcagcctcctaaactgctgatctacggggcatccactagggaatctggggtccctgatcgcttcacaggcagtggatctggaacagatttcactctcaccatcagcagtgtgcaggctgaagacctggcagtttattactgtcagaatgattatagttatcctccgacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctaca

SEQ ID NO: 26 - DNA, 441 bp, synthetic construct
  source 1..441: mol_type = other DNA
atggattttcaagtgcagattttcagcttcctgctaatcagtgcttcagtcaaaatgtccagaggacaaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagtagtaacccaccgacgttcggtggaggcaccaagctggaaatcaaacgggctgatgctgcaccaactgtatccatcttcccaccatccagtgagcagttaaca

SEQ ID NO: 27 - DNA, 489 bp, synthetic construct
  source 1..489: mol_type = other DNA
atggagtcacagactcaggtctttgtatacatgttgctgtggttgtctggtgttgatggagacattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagac

SEQ ID NO: 28 - DNA, 583 bp, synthetic construct
  source 1..583: mol_type = other DNA
atgtcctctgctcagttccttggtctcctgttgctctgttttcaaggtaccagatgtgatatccagatgacacagactacatcctccctgtctgcctctctgggagacagagtcaccatcagttgcagggcaagtcaggatattagcaattatttaaactggtatcagcagaaaccagatggaactgttaaactcctgatctactacacatcaagattacactcaggagtcccatcaaggttcagtggcagtgggtctgggacagattattctctcaccatcagcaacctggaacctgaagatattgccacttactattgtcagcagtatagtaagcttccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatga

SEQ ID NO: 29 - DNA, 657 bp, synthetic construct
  source 1..657: mol_type = other DNA
atggagacagacacactcctgctatgggtgctgctgctctgggttccaggttccactggtgacattgtgctgacacagtctcctgcttccttagctgtatctctatggcagagggccaccatctcatgcagggccagccaaagtgtcagtacatctaggtatagttatatgtactggtaccaacagaaaccaggacagccacccaaactcctcatcaagtatgcatccaacctagaatctggggtccctgccaggttcagtggcagtgggtctgggacagacttcaccctcaacatccatcctgtggaggaggaggatactgcaacatattactgtcagcacagttgggagattccgtacacgttcggaggggggaccatgctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgag

SEQ ID NO: 30 - DNA, 668 bp, synthetic construct
  source 1..668: mol_type = other DNA
atggagacagacacactcctgctatgggtgctgctgctctgggttccaggttccacaggtaacattgtgctgacccaatctccagcttctttggctgtgtctctagggcagagggccaccatatcctgcagagccagtgaaagtgttgatagttatggcaatagttttatgcactggtaccaacagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcaacaaaataatgaggatccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaa

SEQ ID NO: 31 - DNA, 595 bp, synthetic construct
  source 1..595: mol_type = other DNA
atgggatggagctggatctttctctttctcctgtcaggaactgcaggtgtcctctctgaggtccagttgcaacaatctggacctgagctggtgaagcctggggcttcagtgaagatatcctgtaagacttctggatacactttcactgactactacatgaactgggtgaagcagagccatggaaagagccttgagtggattggagatgttaatcctaacaataataatactagatacaatcagaagttcaagggcaaggccacattgaccgtagacaagtcctccagcacagcctacatggagctccgcagcctgacatctgaggactctgcagtctatttctgtgcaaggggcgggctgttctatgctatggacaactggggtcaaggaacctcagtcaccgtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactct

SEQ ID NO: 32 - DNA, 569 bp, synthetic construct
  source 1..569: mol_type = other DNA
atgggatggagctggatctttctcattctcctgtcaggaactgcaggtgtcctctctgaggtccagctgcaacaatctggacctgagctggtgaagcctggggcttcagtgaagatatcctgtaaggcttctggatacacgttcaaagactactacatgaactgggtgaagcagagccatggaaagagccttcagtggattggagatattaatcctaacaatggtggtactacctacaaccagaagttcaagggcaaggccacattgactgtagacaagtcctccagcatagcctacatggagctccgcagcctgacatctgaggactctgcagtctattactgtgcaagcgactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgca

SEQ ID NO: 33 - DNA, 531 bp, synthetic construct
  source 1..531: mol_type = other DNA
atggctgtcctggcactgctcctctgcctggtgacattcccaagctgtgtcctgtcccaggtgcagctgaaggagtcaggacctggcctggtggcgccctcacagagcctgtccatcacatgcactgtctcagggttctcattaaccagctatggtgtaagctgggttcgccagcctccaggaaagggtctggagtggctgggagtaatatggggtgacgggagcacaaattatcattcagctctcatatccagactgagcatcagcaaggataactccaagagccaagttttcttaaaactgaacagtctgcaaactgatgacacagccacgtactactgtgccaaagggggggacggctttgactactggggccaaggcaccactctcacagtctcctcagccaaaacaacagccccatcggtctatccactggcccctgtgtgtggagatacaagtggctcctcggtgactctaggatgcctggtcaagggttatttccctgagccagtgaccttgacctggaactct

SEQ ID NO: 34 - DNA, 499 bp, synthetic construct
  source 1..499: mol_type = other DNA
atgggatggagctggatctttctcctcttcctgtcaggaactgcaggtgtcctctctgaggtccacctgcaacagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtgctatctacaaccagaagttcaagggcaaggccacattgactgtcgacaagtcctccaacacagcctacatggagctccgcagcctgacatctgaggacactgcagtctattactgtgcaagaagggattaccaaagtccctttgactactggggccaaggcaccactctcacagtctcctcagccaaaacaacacccccatcagtctatccactggcccctgggtgtggagatacaactggttcctccgtgactctgggatgcctggtca

SEQ ID NO: 35 - DNA, 595 bp, synthetic construct
  source 1..595: mol_type = other DNA
atgggatggagctggatctttctctttctcctgtcaggaactgcaggtgtcctctctgaggtccagctgcaacaatctggacctgagctggtgaagcctgggacttcaatgaagatatcctgtaaggcttctgcatacacgttcactgactacttcatgaactgggtgaagcagagccatggaaagagccttgagtggattggagatattaatcctaacaatggtgctactaggtacaatcagaggttcaaggtcaaggccacactgactgtagacaagtcctccagcacagcctacatggagctccgcagcctgacatctgaggactctgcagtctattactgtgcaagaggggacgaagggtttgtttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactc

SEQ ID NO: 36 - DNA, 559 bp, synthetic construct
  source 1..559: mol_type = other DNA
atggctgtcttggggctgctcttctgcctggtgacattcccaagctgtgtcctatcccaggtgcagctgaagcagtcaggacctggcctagtgcagccctcacagagcctgtccatcacctgcacagtctctagtttctcattaactagctatggtgtacactgggttcgccagtctccaggaaagggtctggagtggctgggagtgatatggagtggtgtaagcacagactataatgcagctttcatatccagactgaacatcagcaaggacaattccaagagccaagttttctttaaaatgaacagtctgcaaactgatgacacagccatatattactgtgccagaaatgggctcgggctacgtagggcctccatggactactggggtcaaggaacctcagtcaccgtctcctcagccaaaacaacagccccatcggtctatccactggcccctgtgtgtggagatacaagtggctcctcggtgactctaggatgcctggtcaagggttatttccctgagccagtgaccttgacctggaactctggatccctgtcca

SEQ ID NO: 37 - DNA, 580 bp, synthetic construct
  source 1..580: mol_type = other DNA
atggaatggatctggatctttctcttcatcctgtcaggaactgcaggtgtccaatcccaggttcagctgcagcagtctggagctgagctggcgaggcctggggcttcagtgaagctgtcctgcaaggcttctggctacaccttcacaagctatggtataagctgggtgaagcagagaactggacagggccttgagtggattggagagatttatcctagaagtggtaatacttactacaatgagaagttcaagggcaaggccacactgactgcagacaaatcctccagcacagcgtacatggagctccgcagcctgacatctgaggactctgcggtctatttctgtgcaagatcggactactggggccaaggcaccactctcacagtctcctcagccaaaacaacagccccatcggtctatccactggcccctgtgtgtggagatacaagtggctcctcggtgactctaggatgcctggtcaagggttatttccctgagccagtgaccttgacctggaactctggatccctgtccagtggtgtgcacaccttcccagctgtcctgcagtctgacctctaca

SEQ ID NO: 38 - DNA, 523 bp, synthetic construct
  source 1..523: mol_type = other DNA
atgaacttggggctcagcttgattttccttgtccttgttttaaaaggtgtccagtgtgaagtgaagctggtggagtctgggggaggcttagtgcagcctggagggtccctgaaactctcctgtgcagcctctggattcactttcagtgactattacatgtattgggttcgccagactccagagaagaggctggagtgggtcgcatacattagtaatggtggtggtagcacctattatccagacactgtaaagggccgattcaccatctccagagacaatgccaagaacaccctgtacctgcaaatgagccgtctgaagtctgaggacacagccatgtattactgtgcaagactagcggaggatggttactacggggactactttgactactggggccaaggcaccactctcacagtctcctcagccaaaacgacacccccatctgtctatccactggcccctggatctgctgcccaaactaactccatggtgaccctgggatgcctggtcaagggctatttcc

SEQ ID NO: 39 - DNA, 533 bp, synthetic construct
  source 1..533: mol_type = other DNA
atgggatggagctgtatcattgtcctcttggtatcaacagctacaggtgtccactcccaggtccaactgcagcagcctgggtctgaactggtgaggcctgggacttcagtgaagttgtcctgcaaggcttctggctatattttcaccagctactggatgcactgggtaaagcagaggcctggacaaggccttgagtggatcggagtgattgatccttctgatacttatactaactacaatcaaaagttcaagggcaaggccacattgactgtagacacatcctccagcacagcctacatgcagctcagcagcctgacatctgaggactctgcggtctattactgtgcaagagaggagtatggtgactacgtaggaggctttgacttctggggccaaggcaccactctcacagtctcctcagccaaaacaacacccccatcagtctatccactggcccctgggtgtggagatacaactggttcctccgtgactctgggatgcctggtcaagggctacttccctgagtcagtgac

SEQ ID NO: 40 - DNA, 536 bp, synthetic construct
  source 1..536: mol_type = other DNA
atggaatggagctgggtctttctcttcctcctgtcagtaattgcaggtgtccaatcccaggttcaactgcagcagtctggggctgaactggtgaggcctggggcttcagtgacgctgtcctgcaaggcttcgggctacacattttctgactatgaaatgcactgggtgaagcagacacctgtgcatggcctggaatggattggagcttttgatcctgaaactggtactactgcctacaatcagaagttcaaggacaaggccatactgattgcagacaaatcctccagcacagcctacatggagctccgcagcctgacatctgaggactctgccgtctattactgtacaagagcctactataatcactacgttcactttgactactggggccaaggcaccactctcacagtctcctcagccaaaacaacacccccatcagtctatccactggcccctgggtgtggagatacaactggttcctccgtgactctgggatgcctggtcaagggctacttccctgagtcagtgactgtgac

SEQ ID NO: 41 - protein, 25 aa, synthetic construct
EVQLQQSGPELVKSGDLVKMSCKAS

SEQ ID NO: 42 - protein, 25 aa, synthetic construct
EVQLQQSGPELVKPGASVKISCKTS

SEQ ID NO: 43 - protein, 25 aa, synthetic construct
EVHLQQSGPELVKPGASVKIPCKAS

SEQ ID NO: 44 - protein, 25 aa, synthetic construct
QVQLQQSGAELVRPGASVTLSCKAS

SEQ ID NO: 45 - protein, 25 aa, synthetic construct
QVQLQQSGAELAKPGASVKMSCKAS

SEQ ID NO: 46 - protein, 25 aa, synthetic construct
QVQLQQPGAELVKPGASVKLSCKAS

SEQ ID NO: 47 - protein, 25 aa, synthetic construct
QVQLKESGPVLVAPSQSLSITCTVS

SEQ ID NO: 48 - protein, 25 aa, synthetic construct
QVTLKESGPGILQPSQTLSLTCSFS

SEQ ID NO: 49 - protein, 25 aa, synthetic construct
EVKLVESGGGLVKPGGSLNLSCAAS

SEQ ID NO: 50 - protein, 25 aa, synthetic construct
QVQLQQPGAELVKPGASVKLSCKAS

SEQ ID NO: 51 - protein, 25 aa, synthetic construct
EVQLHQSGADLVKPGASVKLSCTAS

SEQ ID NO: 52 - protein, 25 aa, synthetic construct
EVQLHQSGADLVKPGASVKLSCTAS

SEQ ID NO: 53 - protein, 25 aa, synthetic construct
QVQLQQPGAELVKPGASVKLSCKAS

SEQ ID NO: 54 - protein, 25 aa, synthetic construct
QVQLKESGPVLVAPSQSLSITCTVS

SEQ ID NO: 55 - protein, 25 aa, synthetic construct
QVQLQQSGAELARPGASVKMSCKAS

SEQ ID NO: 56 - protein, 25 aa, synthetic construct
QVQLKQSGPGLVQPSQSLSITCTVS

SEQ ID NO: 57 - protein, 25 aa, synthetic construct
QVQLKESGPGLVAPSQSLSITCTVS

SEQ ID NO: 58 - protein, 25 aa, synthetic construct
QVQLQQSGAELARPGASVKLSCKAS

SEQ ID NO: 59 - protein, 25 aa, synthetic construct
EVKLVESGGGLVKPGGSLKLSCAAS

SEQ ID NO: 60 - protein, 25 aa, synthetic construct
EVKLVESGGGLVKPGGSLKLSCAAS

SEQ ID NO: 61 - protein, 25 aa, synthetic construct
QVQLQQPGSELVRPGTSVKLSCKAS

SEQ ID NO: 62 - protein, 25 aa, synthetic construct
EVKLVESGGGLVQPGGSLKLSCAAS

SEQ ID NO: 63 - protein, 25 aa, synthetic construct
EVQLQQSGPELVKPGASVKISCKAS

SEQ ID NO: 64 - protein, 25 aa, synthetic construct
EVQLQQSGPELVKPGTSMKISCKAS

SEQ ID NO: 65 - protein, 8 aa, synthetic construct
GYTFTDYY

SEQ ID NO: 66 - protein, 8 aa, synthetic construct
GYTFTDYY

SEQ ID NO: 67 - protein, 8 aa, synthetic construct
GYTFTDYN

SEQ ID NO: 68 - protein, 8 aa, synthetic construct
GYTFSDYE

SEQ ID NO: 69 - protein, 8 aa, synthetic construct
GYTFSSYW

SEQ ID NO: 70 - protein, 8 aa, synthetic construct
GYTFTSYW

SEQ ID NO: 71 - protein, 8 aa, synthetic construct
GFSLTSYA

SEQ ID NO: 72 - protein, 10 aa, synthetic construct
GFSLSTSGMG

SEQ ID NO: 73 - protein, 8 aa, synthetic construct
GFTFNSFT

SEQ ID NO: 74 - protein, 8 aa, synthetic construct
GYTFTSYW

SEQ ID NO: 75 - protein, 8 aa, synthetic construct
GFHIKDTY

SEQ ID NO: 76 - protein, 8 aa, synthetic construct
GFHIKDTY

SEQ ID NO: 77 - protein, 8 aa, synthetic construct
GYTFTSYW

SEQ ID NO: 78 - protein, 8 aa, synthetic construct
GFSLTSYA

SEQ ID NO: 79 - protein, 8 aa, synthetic construct
GYTFITYT

SEQ ID NO: 80 - protein, 8 aa, synthetic construct
SFSLTSYG

SEQ ID NO: 81 - protein, 8 aa, synthetic construct
GFSLTSYG

SEQ ID NO: 82 - protein, 8 aa, synthetic construct
GYTFTSYG

SEQ ID NO: 83 - protein, 8 aa, synthetic construct
GFTFSGYT

SEQ ID NO: 84 - protein, 8 aa, synthetic construct
GFTFSGYT

SEQ ID NO: 85 - protein, 8 aa, synthetic construct
GYIFTSYW

SEQ ID NO: 86 - protein, 8 aa, synthetic construct
GFTFSDYY

SEQ ID NO: 87 - protein, 8 aa, synthetic construct
GYTFKDYY

SEQ ID NO: 88 - protein, 8 aa, synthetic construct
AYTFTDYF

SEQ ID NO: 89 - protein, 17 aa, synthetic construct
MDWVKQSHGKSLEWIGY

SEQ ID NO: 90 - protein, 17 aa, synthetic construct
MNWVKQSHGKSLEWIGD

SEQ ID NO: 91 - protein, 17 aa, synthetic construct
MDWVKQSHGKSLEWIGD

SEQ ID NO: 92 - protein, 17 aa, synthetic construct
MHWVKQTPVHGLEWIGA

SEQ ID NO: 93 - protein, 17 aa, synthetic construct
MHWVKQRPGQDLEWIGY

SEQ ID NO: 94 - protein, 17 aa, synthetic construct
MHWVKQRPGQGLEWIGN

SEQ ID NO: 95 - protein, 17 aa, synthetic construct
IHWVRQPPGKGLEWLGV

SEQ ID NO: 96 - protein, 17 aa, synthetic construct
VSWIRQPSGKGLEWLAH

SEQ ID NO: 97 - protein, 17 aa, synthetic construct
MSWVRQTPAKRLEWVAT

SEQ ID NO: 98 - protein, 17 aa, synthetic construct
MHWVKQRPGQGLEWIGN

SEQ ID NO: 99 - protein, 17 aa, synthetic construct
IHWMKQRPEQGLEWIGR

SEQ ID NO: 100 - protein, 17 aa, synthetic construct
IHWMKQRPEQGLEWIGR

SEQ ID NO: 101 - protein, 17 aa, synthetic construct
MHWVKQRPGQGLEWIGN

SEQ ID NO: 102 - protein, 17 aa, synthetic construct
IHWVRQPPGKGLEWLGV

SEQ ID NO: 103 - protein, 17 aa, synthetic construct
MHWVKQRPGQGLEWIGY

SEQ ID NO: 104 - protein, 17 aa, synthetic construct
VHWVRQSPGKGLEWLGV

SEQ ID NO: 105 - protein, 17 aa, synthetic construct
VSWVRQPPGKGLEWLGV

SEQ ID NO: 106 - protein, 17 aa, synthetic construct
ISWVKQRTGQGLEWIGE

SEQ ID NO: 107 - protein, 17 aa, synthetic construct
MSWVRQTPAKRLEWVAT

SEQ ID NO: 108 - protein, 17 aa, synthetic construct
MSWVRQTPAKRLEWVAT

SEQ ID NO: 109 - protein, 17 aa, synthetic construct
MHWVKQRPGQGLEWIGV

SEQ ID NO: 110 - protein, 17 aa, synthetic construct
MYWVRQTPEKRLEWVAY

SEQ ID NO: 111 - protein, 17 aa, synthetic construct
MNWVKQSHGKSLQWIGD

SEQ ID NO: 112 - protein, 17 aa, synthetic construct
MNWVKQSHGKSLEWIGD

SEQ ID NO: 113 - protein, 8 aa, synthetic construct
IYPNNGGT

SEQ ID NO: 114 - protein, 8 aa, synthetic construct
VNPNNNNT

SEQ ID NO: 115 - protein, 8 aa, synthetic construct
INPNNGGA

SEQ ID NO: 116 - protein, 8 aa, synthetic construct
FDPETGTT

SEQ ID NO: 117 - protein, 8 aa, synthetic construct
IDPSSSYA

SEQ ID NO: 118 - protein, 8 aa, synthetic construct
IDPSDSET

SEQ ID NO: 119 - protein, 7 aa, synthetic construct
IWAGGST

SEQ ID NO: 120 - protein, 7 aa, synthetic construct
IYWDDDK

SEQ ID NO: 121 - protein, 8 aa, synthetic construct
ISSSGYNT

SEQ ID NO: 122 - protein, 8 aa, synthetic construct
IDPSDSET

SEQ ID NO: 123 - protein, 8 aa, synthetic construct
IDPANGNS

SEQ ID NO: 124 - protein, 8 aa, synthetic construct
IDPANGNS

SEQ ID NO: 125 - protein, 8 aa, synthetic construct
IDPSDSET

SEQ ID NO: 126 - protein, 7 aa, synthetic construct
IWAGGST

SEQ ID NO: 127 - protein, 8 aa, synthetic construct
INPSSDYT

SEQ ID NO: 128 - protein, 7 aa, synthetic construct
IWSGVST

SEQ ID NO: 129 - protein, 7 aa, synthetic construct
IWGDGST

SEQ ID NO: 130 - protein, 8 aa, synthetic construct
IYPRSGNT

SEQ ID NO: 131 - protein, 8 aa, synthetic construct
ISGSGGNI

SEQ ID NO: 132 - protein, 8 aa, synthetic construct
ISGSGGNI

SEQ ID NO: 133 - protein, 8 aa, synthetic construct
IDPSDTYT

SEQ ID NO: 134 - protein, 8 aa, synthetic construct
ISNGGGST

SEQ ID NO: 135 - protein, 8 aa, synthetic construct
INPNNGGT

SEQ ID NO: 136 - protein, 49 aa, synthetic construct
INPNNGATREFSYNQKFKGKATLTVDKSSSTAYMELHSLTSEDSAVYYC

SEQ ID NO: 137 - protein, 38 aa, synthetic construct
RYNQKFKGKATLTVDKSSSTAYMELRSLTSEDSAVYFC

SEQ ID NO: 138 - protein, 38 aa, synthetic construct
IYNQKFKGKATLTVDKSSNTAYMELRSLTSEDTAVYYC

SEQ ID NO: 139 - protein, 38 aa, synthetic construct
AYNQKFKDKAILIADKSSSTAYMELRSLTSEDSAVYYC

SEQ ID NO: 140 - protein, 38 aa, synthetic construct
EYNQKFKDKATLTADRSSSTAYMQLSSLTSEDSAVYYC

SEQ ID NO: 141 - protein, 38 aa, synthetic construct
HYNQKFKDKATLTVDKSSSTAYMQLSSLTSEDSAVYYC

SEQ ID NO: 142 - protein, 38 aa, synthetic construct
DYNSALMSRLNINKDNSKSQVFLKMNSLQIDDTAMYYC

SEQ ID NO: 143 - protein, 38 aa, synthetic construct
RYNPSLKSRLTISKDTSSNQVFLKITSVDTADTATYYC

SEQ ID NO: 144 - protein, 38 aa, synthetic construct
YYPDSVKGRFTISRDNARNTLYLQMSSLRSEDTAMYYC

SEQ ID NO: 145 - protein, 38 aa, synthetic construct; identical to SEQ ID NO: 141

SEQ ID NO: 146 - protein, 38 aa, synthetic construct
EYDPKFQGKATITADTSSNTAYLHLSSLTSEDTAVYYC

SEQ ID NO: 147 - protein, 38 aa, synthetic construct; identical to SEQ ID NO: 146

SEQ ID NO: 148 - protein, 38 aa, synthetic construct; identical to SEQ ID NO: 141

SEQ ID NO: 149 - protein, 38 aa, synthetic construct; identical to SEQ ID NO: 142

SEQ ID NO: 150 - protein, 38 aa, synthetic construct
NYNQRFKDKATLTADKSSSTAYMQLNSLTSEDSAVYYC

SEQ ID NO: 151 - protein, 38 aa, synthetic construct
DYNAAFISRLNISKDNSKSQVFFKMNSLQTDDTAIYYC

SEQ ID NO: 152 - protein, 38 aa, synthetic construct
NYHSALISRLSISKDNSKSQVFLKLNSLQTDDTATYYC

SEQ ID NO: 153 - protein, 38 aa, synthetic construct
YYNEKFKGKATLTADKSSSTAYMELRSLTSEDSAVYFC

SEQ ID NO: 154 - protein, 38 aa, synthetic construct
YYPDTVKGRFTISRDNARNTLYLQMSSLRSEDTAMYYC

SEQ ID NO: 155 - protein, 38 aa, synthetic construct; identical to SEQ ID NO: 154

SEQ ID NO: 156 - protein, 38 aa, synthetic construct
NYNQKFKGKATLTVDTSSSTAYMQLSSLTSEDSAVYYC

SEQ ID NO: 157 - protein, 38 aa, synthetic construct
YYPDTVKGRFTISRDNAKNTLYLQMSRLKSEDTAMYYC

SEQ ID NO: 158 - protein, 38 aa, synthetic construct
TYNQKFKGKATLTVDKSSSIAYMELRSLTSEDSAVYYC

SEQ ID NO: 159 - protein, 38 aa, synthetic construct
RYNQRFKVKATLTVDKSSSTAYMELRSLTSEDSAVYYC

SEQ ID NO: 160 - protein, 5 aa, synthetic construct
ARGAY

SEQ ID NO: 161 - protein, 11 aa, synthetic construct
ARGGLFYAMDN

SEQ ID NO: 162 - protein, 11 aa, synthetic construct
ARRDYQSPFDY

SEQ ID NO: 163 - protein, 13 aa, synthetic construct
TRAYYNHYVHFDY

SEQ ID NO: 164 - protein, 14 aa, synthetic construct
VRGYFGNYDYALDY

SEQ ID NO: 165 - protein, 11 aa, synthetic construct
ARHYDGYAVDY

SEQ ID NO: 166 - protein, 15 aa, synthetic construct
ARHSSKAYYSNYFDY

SEQ ID NO: 167 - protein, 13 aa, synthetic construct
ARTMITTRNYFDY

SEQ ID NO: 168 - protein, 16 aa, synthetic construct
ARHGKIYDGYPFAMDF

SEQ ID NO: 169 - protein, 11 aa, synthetic construct
ARHYDGYAVDY

SEQ ID NO: 170 - protein, 15 aa, synthetic construct
AESNYFGSSPYALDY

SEQ ID NO: 171 - protein, 15 aa, synthetic construct
AESNYFGSSPYALDY

SEQ ID NO: 172 - protein, 11 aa, synthetic construct
ARHYDGYAVDY

SEQ ID NO: 173 - protein, 15 aa, synthetic construct
ARHSSKAYYSNYFDY

SEQ ID NO: 174 - protein, 7 aa, synthetic construct
ARGAPAY

SEQ ID NO: 175 - protein, 14 aa, synthetic construct
ARNGLGLRRASMDY

SEQ ID NO: 176 - protein, 9 aa, synthetic construct
AKGGDGFDY

SEQ ID NO: 177 - protein, 5 aa, synthetic construct
ARSDY

SEQ ID NO: 178 - protein, 12 aa, synthetic construct
TRQEGGLRGFAY

SEQ ID NO: 179 - protein, 12 aa, synthetic construct
TRQEGGLRGFAY

SEQ ID NO: 180 - protein, 14 aa, synthetic construct
AREEYGDYVGGFDF

SEQ ID NO: 181 - protein, 15 aa, synthetic construct
ARLAEDGYYGDYFDY

SEQ ID NO: 182 - protein, 6 aa, synthetic construct
ASDFDY

SEQ ID NO: 183 - protein, 9 aa, synthetic construct
ARGDEGFVY

SEQ ID NO: 184 - protein, 11 aa, synthetic construct
WGQGTLVTVSA

SEQ ID NO: 185 - protein, 11 aa, synthetic construct
WGQGTSVTVSS

SEQ ID NO: 186 - protein, 11 aa, synthetic construct
WGQGTTLTVSS

SEQ ID NO: 187 - protein, 11 aa, synthetic construct
WGQGTTLTVSS

SEQ ID NO: 188 - protein, 11 aa, synthetic construct
WGQGTSVTVSS

SEQ ID NO: 189 - protein, 11 aa, synthetic construct
WGQGTSVTVSS

SEQ ID NO: 190 - protein, 11 aa, synthetic construct
WGQGTTLTVSS

SEQ ID NO: 191 - protein, 11 aa, synthetic construct
WGQGTTLTVSS

SEQ ID NO: 192 - protein, 11 aa, synthetic construct
WGQGTSVTVSS

SEQ ID NO: 193 - protein, 11 aa, synthetic construct
WGQGTSVTVSS

SEQ ID NO: 194 - protein, 11 aa, synthetic construct
WGQGTSVTVSS

SEQ ID NO: 195 - protein, 11 aa, synthetic construct
WGQGTSVTVSS

SEQ ID NO: 196 - protein, 11 aa, synthetic construct
WGQGTSVTVSS

SEQ ID NO: 197 - protein, 11 aa, synthetic construct
WGQGTTLTVSS

SEQ ID NO: 198 - protein, 11 aa, synthetic construct
WGQGTLVTVSA

SEQ ID NO: 199 - protein, 11 aa, synthetic construct
WGQGTSVTVSS

SEQ ID NO: 200 - protein, 11 aa, synthetic construct
WGQGTTLTVSS

SEQ ID NO: 201 - protein, 11 aa, synthetic construct
WGQGTTLTVSS

SEQ ID NO: 202 - protein, 11 aa, synthetic construct
WGQGTLVTVSA

SEQ ID NO: 203 - protein, 11 aa, synthetic construct
WGQGTLVTVSA

SEQ ID NO: 204 - protein, 11 aa, synthetic construct
WGQGTTLTVSS

SEQ ID NO: 205 - protein, 11 aa, synthetic construct
WGQGTTLTVSS

SEQ ID NO: 206 - protein, 11 aa, synthetic construct
WGQGTTLTVSS

SEQ ID NO: 207 - protein, 11 aa, synthetic construct
WGQGTLVTVSA

SEQ ID NO: 208 - protein, 26 aa, synthetic construct
DIVMTQSQKFMSTSVGDRVSVTCKAS

SEQ ID NO: 209 - protein, 26 aa, synthetic construct
DIVMTQSPSSLSVSAGHKVTMSCKSS

SEQ ID NO: 210 - protein, 26 aa, synthetic construct
DIQMTQTTSSLSASLGDRVTISCSAS

SEQ ID NO: 211 - protein, 26 aa, synthetic construct
NIVLTQSPASLAVSLGQRATISCRAS

SEQ ID NO: 212 - protein, 26 aa, synthetic construct [sequence omitted]

SEQ ID NO: 213 - protein, 26 aa, synthetic construct
NIVLTQSPASLAVSLGQRATISCRAS

SEQ ID NO: 214 - protein, 26 aa, synthetic construct
DIVLTQSPASLPVSLGQRATISCRAS

SEQ ID NO: 215 - protein, 26 aa, synthetic construct
DIQITQTTSSLSASLGDRVTISCSAS

SEQ ID NO: 216 - protein, 26 aa, synthetic construct
DIVLTQSPASLAVSLGQRATISCKAS

SEQ ID NO: 217 - protein, 26 aa, synthetic construct
DIQMTQSPASLSVSVGETVTITCRAS

SEQ ID NO: 218 - protein, 26 aa, synthetic construct
DIVLTQSPASLPVSLGQRATISCRAS

SEQ ID NO: 219 - protein, 26 aa, synthetic construct
DIILTQSPATLSVTPGDSVSLSCRAS

SEQ ID NO: 220 - protein, 26 aa, synthetic construct
DIVMTQSHKFMSTSVGDRVSISCKAS

SEQ ID NO: 221 - protein, 26 aa, synthetic construct
DIVLTQSPASLPVSLGQRATISCRAS

SEQ ID NO: 222 - protein, 26 aa, synthetic construct
DIQITQTTSSLSASLGDRVTISCSAS

SEQ ID NO: 223 - protein, 26 aa, synthetic construct
DIILTQSPATLSVTPGDSVSLSCRAS

SEQ ID NO: 224 - protein, 26 aa, synthetic construct [sequence omitted]

SEQ ID NO: 225 - protein, 26 aa, synthetic construct
DIVLTQSPASLAVSLGQRATISCRAS

SEQ ID NO: 226 - protein, 26 aa, synthetic construct
DIVMTQSQKFMSTSVGDRVSVTCKAS

SEQ ID NO: 227 - protein, 26 aa, synthetic construct
DIVLTQSPASLAVSLGQRATISCRAS

SEQ ID NO: 228 - protein, 26 aa, synthetic construct
DIQMTQSPASLSVSVGETVTITCRAS

SEQ ID NO: 229 - protein, 26 aa, synthetic construct
DIQMTQSPASLSVSVGETVTITCRAS

SEQ ID NO: 230 - protein, 26 aa, synthetic construct
DIVLTQSPASLAVSLWQRATISCRAS

SEQ ID NO: 231 - protein, 26 aa, synthetic construct
DIQMTQTTSSLSASLGDRVTISCRAS

SEQ ID NO: 232 - protein, 26 aa, synthetic construct
DVVMTQTPLSLPVSLGDQASISCRSS

SEQ ID NO: 233 - protein, 26 aa, synthetic construct
DIVMTQSPSSLSVSAGDKVTMSCKSS

SEQ ID NO: 234 - protein, 6 aa, synthetic construct
QNVGTN

SEQ ID NO: 235 - protein, 12 aa, synthetic construct
QSLLNSRNQKNY

SEQ ID NO: 236 - protein, 6 aa, synthetic construct
QGINNY

SEQ ID NO: 237 - protein, 10 aa, synthetic construct
ESVDSYGNSF

SEQ ID NO: 238 - protein, 5 aa, synthetic construct
SSVSY

SEQ ID NO: 239 - protein, 10 aa, synthetic construct
ESVDSYGNSF

SEQ ID NO: 240 - protein, 10 aa, synthetic construct
QSVTTSRYSY

SEQ ID NO: 241 - protein, 6 aa, synthetic construct
QGLSNY

SEQ ID NO: 242 - protein, 10 aa, synthetic construct
QSVDYDGDSY

SEQ ID NO: 243 - protein, 6 aa, synthetic construct
ENIYSN

SEQ ID NO: 244 - protein, 10 aa, synthetic construct
QSVTTSRYSY

SEQ ID NO: 245 - protein, 6 aa, synthetic construct
QSISNN

SEQ ID NO: 246 - protein, 6 aa, synthetic construct
QDVGTA

SEQ ID NO: 247 - protein, 10 aa, synthetic construct
QSVTTSRYSY

SEQ ID NO: 248 - protein, 6 aa, synthetic construct
QGLSNY

SEQ ID NO: 249 - protein, 6 aa, synthetic construct
QSISNN

SEQ ID NO: 250 - protein, 5 aa, synthetic construct
SSVSY

SEQ ID NO: 251 - protein, 10 aa, synthetic construct
ESVSIRGTSL

SEQ ID NO: 252 - protein, 6 aa, synthetic construct
QNVGTN

SEQ ID NO: 253 - protein, 10 aa, synthetic construct
ESVDNYGISF

SEQ ID NO: 254 - protein, 6 aa, synthetic construct
ENIYNN

SEQ ID NO: 255 - protein, 6 aa, synthetic construct
ENIYNN

SEQ ID NO: 256 - protein, 10 aa, synthetic construct
QSVSTSRYSY

SEQ ID NO: 257 - protein, 6 aa, synthetic construct
QDISNY

SEQ ID NO: 258 - protein, 11 aa, synthetic construct
QSLVYSNGNTY

SEQ ID NO: 259 - protein, 12 aa, synthetic construct
QSLLNSRNQKNY

SEQ ID NO: 260 - protein, 17 aa, synthetic construct
VAWYQQKPGQSPKALIY

SEQ ID NO: 261 - protein, 17 aa, synthetic construct
LAWYQQKPWQPPKLLIY

SEQ ID NO: 262 - protein, 17 aa, synthetic construct
LNWYQQKPDGTVKLLIY

SEQ ID NO: 263 - protein, 17 aa, synthetic construct
MHWYQQKPGQPPKLLIY

SEQ ID NO: 264 - protein, 17 aa, synthetic construct
MHWYQQKPGSSPKPWIY

SEQ ID NO: 265 - protein, 17 aa, synthetic construct
MHWYQQKPGQPPKLLIY

SEQ ID NO: 266 - protein, 17 aa, synthetic construct
MHWYQQKPGQPPKLLIM

SEQ ID NO: 267 - protein, 17 aa, synthetic construct
LNWYQQKPDGTVKLLIY

SEQ ID NO: 268 - protein, 17 aa, synthetic construct
MNWYQQKPGQPPKLLIY

SEQ ID NO: 269 - protein, 17 aa, synthetic construct
LAWYHQKQGKSPQLLVY

SEQ ID NO: 270 - protein, 17 aa, synthetic construct
MHWYQQKPGQPPKLLIM

SEQ ID NO: 271 - protein, 17 aa, synthetic construct
LHWYQQKSHESPRLLIK

SEQ ID NO: 272 - protein, 17 aa, synthetic construct
VAWYQHKPGQSPKVLIY

SEQ ID NO: 273 - protein, 17 aa, synthetic construct
MHWYQQKPGQPPKLLIM

SEQ ID NO: 274 - protein, 17 aa, synthetic construct
LNWYQQKPDGTVKLLIY

SEQ ID NO: 275 - protein, 17 aa, synthetic construct
LHWYQQKSHESPRLLIK

SEQ ID NO: 276 - protein, 17 aa, synthetic construct
MHWYQQKPGSSPKPWIY

SEQ ID NO: 277 - protein, 17 aa, synthetic construct
MHWYQQKPGYPPKLLIY

SEQ ID NO: 278 - protein, 17 aa, synthetic construct
VAWYQQKPGQSPKALIY

SEQ ID NO: 279 - protein, 17 aa, synthetic construct
MNWFQQKPGQPPKLLIY

SEQ ID NO: 280 - protein, 17 aa, synthetic construct
LAWYQQKQGKSPQLLVY

SEQ ID NO: 281 - protein, 17 aa, synthetic construct
LAWYQQKQGKSPQLLVY

SEQ ID NO: 282 - protein, 17 aa, synthetic construct
MYWYQQKPGQPPKLLIK

SEQ ID NO: 283 - protein, 17 aa, synthetic construct
LNWYQQKPDGTVKLLIY

SEQ ID NO: 284 - protein, 17 aa, synthetic construct
LHWYLQKPGQSPKLLIY

SEQ ID NO: 285 - protein, 17 aa, synthetic construct
LAWYQQKPWQPPKLLIY

SEQ ID NO: 286-301 - skipped (16 entries)

SEQ ID NO: 302 - protein, 27 aa, synthetic construct
ATSREFAASREFSASREFAASREFAAT

SEQ ID NO: 303-307 - skipped (5 entries)

SEQ ID NO: 308 - protein, 36 aa, synthetic construct
YRYSGVPDRFTGSGSGTDFTLTISNVQSEDLAEYFC

SEQ ID NO: 309 - protein, 36 aa, synthetic construct
TRESGVPDRFTGSGSGTDFTLTISSVQAEDLAVYYC

SEQ ID NO: 310 - protein, 270 aa, synthetic construct
SLHSGVPSRFSGSGSGTDYSLTISNLEPEDIATYYCREFNLESGVPARFSGSGSRTDFTLTIDPVEADDAATYYCREFNLASGVPARFSGSGSGTSYSLTISRVEAEDAATYYCREFNLESGVPARFSGSGSRTDFTLTIDPVEADDAATYYCREFNLESGVPARFSGSGSGTDFTLNIHPVEEEDIAIYYCREFILHSGVPSRFSGSGSGTDYSLTISNLEPEDIATYYCREFNLESGIPARFSGSGSGTDFTLNIHPVEEEDAATYYC

SEQ ID NO: 311 - protein, 36 aa, synthetic construct
NLADGVPSRFSGNGSGTQYSLKINSLQSEDFGSYYC

SEQ ID NO: 312 - protein, 36 aa, synthetic construct
NLESGVPARFSGSGSGTDFTLNIHPVEEEDIAIYYC

SEQ ID NO: 313 - protein, 36 aa, synthetic construct
QSISGIPSRFSGSGSGTDFTLSINSVETEDFGMYFC

SEQ ID NO: 314 - protein, 36 aa, synthetic construct
NRHTGVPDRFTGSGSGTDFTLTITNVQSEDLADYFC

SEQ ID NO: 315 - protein, 36 aa, synthetic construct; identical to SEQ ID NO: 312

SEQ ID NO: 316 - protein, 36 aa, synthetic construct
ILHSGVPSRFSGSGSGTDYSLTISNLEPEDIATYYC

SEQ ID NO: 317 - protein, 36 aa, synthetic construct; identical to SEQ ID NO: 313

SEQ ID NO: 318 - protein, 36 aa, synthetic construct [sequence omitted]

SEQ ID NO: 319 - protein, 36 aa, synthetic construct
NLESGVPARFSGRRSGTDFTLNIHPVEEDDAATYYC

SEQ ID NO: 320 - protein, 36 aa, synthetic construct; identical to SEQ ID NO: 308

SEQ ID NO: 321 - protein, 36 aa, synthetic construct
NQGSGVPARFSGSGSGTDFSLNIHPMEEDDTAMYFC

SEQ ID NO: 322 - protein, 36 aa, synthetic construct
NLADGVPSRFSGSGSGTQFSLKINSLQSEDFGSYYC

SEQ ID NO: 323 - protein, 36 aa, synthetic construct; identical to SEQ ID NO: 322

SEQ ID NO: 324 - protein, 36 aa, synthetic construct
NLESGVPARFSGSGSGTDFTLNIHPVEEEDTATYYC

SEQ ID NO: 325 - protein, 36 aa, synthetic construct
RLHSGVPSRFSGSGSGTDYSLTISNLEPEDIATYYC

SEQ ID NO: 326 - protein, 36 aa, synthetic construct [sequence omitted]

SEQ ID NO: 327 - protein, 36 aa, synthetic construct; identical to SEQ ID NO: 309

SEQ ID NO: 328 - protein, 9 aa, synthetic construct
QQYNNYPYT

SEQ ID NO: 329 - protein, 9 aa, synthetic construct
QNDYSYPPT

SEQ ID NO: 330 - protein, 9 aa, synthetic construct
QQYSKLPFT

SEQ ID NO: 331 - protein, 9 aa, synthetic construct
QQNNEDPWT

SEQ ID NO: 332 - protein, 9 aa, synthetic construct
QQWSSNPPT

SEQ ID NO: 333 - protein, 9 aa, synthetic construct
QQNNEDPWT

SEQ ID NO: 334 - protein, 9 aa, synthetic construct
QHSWEIPWT

SEQ ID NO: 335 - protein, 9 aa, synthetic construct
XQYSKLPWT

SEQ ID NO: 336 - protein, 8 aa, synthetic construct
QQSNEDPP

SEQ ID NO: 337 - protein, 9 aa, synthetic construct
QHFWGTPWT

SEQ ID NO: 338 - protein, 9 aa, synthetic construct
QHSWEIPWT

SEQ ID NO: 339 - protein, 9 aa, synthetic construct
QQSNSWPLT

SEQ ID NO: 340 - protein, 8 aa, synthetic construct
QQYSYYPT

SEQ ID NO: 341 - protein, 9 aa, synthetic construct
QHSWEIPWT

SEQ ID NO: 342 - protein, 9 aa, synthetic construct
QQYSKLPWT

SEQ ID NO: 343 - protein, 9 aa, synthetic construct
QQSNSWPLT

SEQ ID NO: 344 - protein, 9 aa, synthetic construct
QQWSSNPPT

SEQ ID NO: 345 - protein, 9 aa, synthetic construct
QQSREYPLT

SEQ ID NO: 346 - protein, 9 aa, synthetic construct
QQYNSYPYT

SEQ ID NO: 347 - protein, 9 aa, synthetic construct
QQSKEVPFT

SEQ ID NO: 348 - protein, 9 aa, synthetic construct
QHFWGIPYT

SEQ ID NO: 349 - protein, 9 aa, synthetic construct
QHFWGIPYT

SEQ ID NO: 350 - protein, 9 aa, synthetic construct
QHSWEIPYT

SEQ ID NO: 351 - protein, 9 aa, synthetic construct
QQYSKLPLT

SEQ ID NO: 352 - protein, 9 aa, synthetic construct
SQSTHVPFT

SEQ ID NO: 353 - protein, 9 aa, synthetic construct
QNDYSYPPT

SEQ ID NO: 354 - protein, 10 aa, synthetic construct
FGGGTKLEIK

SEQ ID NO: 355 - protein, 10 aa, synthetic construct
FGAGTKLELK

SEQ ID NO: 356 - protein, 10 aa, synthetic construct
FGSGTKLEIK

SEQ ID NO: 357 - protein, 10 aa, synthetic construct
FGGGTKLEIK

SEQ ID NO: 358 - protein, 10 aa, synthetic construct
FGGGTKLEIK

SEQ ID NO: 359 - protein, 10 aa, synthetic construct
FGGGTKLEIK

SEQ ID NO: 360 - protein, 10 aa, synthetic construct
FGGGTKLEIK

SEQ ID NO: 361 - protein, 10 aa, synthetic construct
FGGGSKLEIK

SEQ ID NO: 362 - protein, 10 aa, synthetic construct
FGGGTKLEIK

SEQ ID NO: 363 - protein, 10 aa, synthetic construct
FGGGTKLEIK

SEQ ID NO: 364 - protein, 10 aa, synthetic construct
FGGGTKLEIK

SEQ ID NO: 365 - protein, 10 aa, synthetic construct
FGAGTKLELK

SEQ ID NO: 366 - protein, 10 aa, synthetic construct
FGGGTKLEIK

SEQ ID NO: 367 - protein, 10 aa, synthetic construct
FGGGTKLEIK

SEQ ID NO: 368 - protein, 10 aa, synthetic construct
FGGGSKLEIK

SEQ ID NO: 369 - protein, 10 aa, synthetic construct
FGAGTKLELK

SEQ ID NO: 370 - protein, 10 aa, synthetic construct
FGGGTKLEIK

SEQ ID NO: 371 - protein, 10 aa, synthetic construct
FGAGTKLELK

SEQ ID NO: 372 - protein, 10 aa, synthetic construct
FGGGTKLEIK

SEQ ID NO: 373 - protein, 10 aa, synthetic construct
FGSGTKLEIK

SEQ ID NO: 374 - protein, 10 aa, synthetic construct
FGGGTKLEIT

SEQ ID NO: 375 - protein, 10 aa, synthetic construct
FGGGTKLEIT

SEQ ID NO: 376 - protein, 10 aa, synthetic construct
FGGGTMLEIK

SEQ ID NO: 377 - protein, 10 aa, synthetic construct
FGAGTKLELK

SEQ ID NO: 378 - protein, 10 aa, synthetic construct
FGSGTKLEIK

SEQ ID NO: 379 - protein, 10 aa, synthetic construct
FGGGTKLEIK